(19) United States
(12) Patent Application Publication
(10) Pub. No.: US 2023/0233533 A1
(43) Pub. Date: Jul. 27, 2023

(54) COMPOSITIONS, METHODS OF TREATING AND PREVENTING FUNGAL INFECTIONS, AND METHODS OF INHIBITING PRP8 INTEIN EXPRESSION

(71) Applicants: Health Research, Inc., Menands, NY (US); The Board of Regents of the University of Texas System, Austin, TX (US)

(72) Inventors: Hongmin LI, Tucson, AZ (US); Zhong LI, Tucson, AZ (US); Jia ZHOU, League City, TX (US); Jimin XU, Galveston, TX (US)

(73) Assignees: Health Research, Inc., Menands, NY (US); The Board of Regents of the University of Texas System, Austin, TX (US)

(21) Appl. No.: 18/001,316

(22) PCT Filed: Apr. 9, 2021
(86) PCT No.: PCT/US2021/026648

§ 371 Date: Dec. 9, 2022

Related U.S. Application Data
(60) Provisional application No. 63/066,518, filed on Aug. 17, 2020.
(60) Provisional application No. 63/037,326, filed on Jun. 10, 2020.

(51) Int. Cl. A61K 31/426; A61P 31/10; A61K 31/4439; A61K 31/433; A61K 45/06; G01N 33/68

(52) U.S. Cl. CPC A61K 31/426; A61P 31/10; A61K 31/4439; A61K 31/433; A61K 45/06; G01N 33/6893

(57) ABSTRACT

The present disclosure relates to a Prp8 intein splicing inhibitor. The present disclosure further relates to a method of treating and/or preventing a fungal infection, said method comprising administering a Prp8 intein splicing inhibitor under conditions effective to treat and/or prevent a fungal infection. Also disclosed is a method of inhibiting Prp8 intein expression or activity in a cell or tissue, said method comprising administering a compound under conditions effective to inhibit Prp8 intein expression or activity in a cell or tissue. Further disclosed are methods for screening for compounds that inhibit Prp8 intein splicing comprising an assay and a kit for predicting the likelihood of Prp8 inhibition.

This application claims benefit of U.S. Provisional Patent Application Serial Nos. 63/037,326, filed Jun. 10, 2020, and 63/066,518, filed Aug. 17, 2020, both of which are hereby incorporated by reference in their entirety.

This invention was made with government support under AI140726, AI141178, and GM44844 awarded by the National Institutes of Health. The government has certain rights in the invention.

### SEQUENCE LISTING

The present application contains a Sequence Listing, created on Apr. 8, 2021; the file, in ASCII format, is designated as 0332089AWO_sequencelisting_ST25.txt and is 2.31 kilobytes in size. The file is hereby incorporated by reference in its entirety into the instant application.

### FIELD

The present disclosure relates generally to compositions, methods of treating and preventing fungal infections, and methods of inhibiting Prp8 intein expression.

### BACKGROUND

Many microbial pathogens contain self-splicing elements called inteins, which are internal proteins that self-excise from their intein-hosting proteins and catalyze ligation of the flanking sequences (exteins) with a natural peptide bond. Mills et al., “Protein Splicing: How Inteins Escape from Precursor Proteins,” The Journal of Biological Chemistry 289(21):14498-14505 (2014); Aranko et al., “Nature's Recipe for Splitting Inteins,” Protein Engineering, Design & Selection 27(8):263-271 (2014); Eryilmaz et al., “Structural and Dynamical Features of Inteins and Implications on Protein Splicing,” The Journal of Biological Chemistry 289(21):14506-14511 (2014); and Novikova et al., “Enigmatic Distribution, Evolution, and Function of Inteins,” The Journal of Biological Chemistry 289(21):14490-14497 (2014). Overall, more than 1,700 inteins have been identified. Novikova et al., “Intein Clustering Suggests Functional Importance in Different Domains of Life,” Molecular Biology and Evolution 33(3):783-799 (2016). Among intein-containing deadly human pathogens is Mycobacterium tuberculosis (Mtu), which has inteins in three critical genes, involved in replication (DnaB), iron-sulfur cluster assembly (SufB), and recombination (RecA). M. tuberculosis infections cause 2 million annual tuberculosis-related deaths worldwide. Center for Disease Control and Prevention, “Basic TB Facts,” Division of Tuberculosis Elimination (2016). Pathogenic fungi such as Crypotococcus neoformans (Cne), Crypotococcus gattii (Cga), and Aspergillus fumigatus (Afu) also encode inteins, in the pre-mRNA processing factor 8 (Prp8) gene. Green et al., “Spliceosomal Prp8 Intein at the Crossroads of Protein and RNA Splicing,” PLoS Biol 17(10):e3000104 (2019) and Li et al., “Cisplatin Protects Mice From Challenge of Cryptococcus neoformans by Targeting the Prp8 Intein,” Emerging Microbes & Infections 8(1):895-908 (2019). Globally, over 300 million people are affected by invasive fungal infections (IFIs), with estimated deaths of over 1 million people every year. Perfect, John R. “Fungal Diagnosis: How Do We Do It and Can We Do Better?” Current Medical Research and Opinion 29 Suppl 4:3-11 (2013); Brown et al., “Hidden Killers: Human Fungal Infections,” Science Translational Medicine 4(165):165rv113 (2012); Gullo, Antonino “Invasive Fungal Infections: The Challenge Continues,” Drugs 69 Suppl 1:65-73 (2009); and Tuite, N. & Lacey, K., “Overview of Invasive Fungal Infections,” Methods in Molecular Biology 968:1-23 (2013). Moreover, the emergence of severely drug-resistant strains of M. tuberculosis and pathogenic fungi, plus the deadly synergistic association with HIV/AIDS, represent significant public health challenges. Gandhi et al., “Extensively Drug-Resistant Tuberculosis as a Cause of Death in Patients Co-Infected with Tuberculosis and HIV in a Rural Area of South Africa,” Lancet 368(9547):1575-1580 (2006); World Health Organization “Management of MDR-TB: A Field Guide: A Companion Document to Guidelines for Programmatic Management of Drug-Resistant Tuberculosis: Integrated Management of Adolescent and Adult Illness (IMAI),” World Health Organization (2009); Nathan, Carl “Taming Tuberculosis: A Challenge for Science and Society,” Cell Host & Microbe 5(3):220-224(2009); Pfaller, Michael A. “Antifungal Drug Resistance: Mechanisms, Epidemiology, and Consequences for Treatment,” The American Journal of Medicine 125 (1 Suppl):S3-13 (2012); Ghannoum, M. A. & Rice, L. B. “Antifungal Agents: Mode of Action, Mechanisms of Resistance, and Correlation of These Mechanisms with Bacterial Resistance,” Clinical Microbiology Reviews 12(4):501-517 (1999); and Howard et al., “Frequency and Evolution of Azole Resistance in Aspergillus Fumigatus Associated with Treatment Failure,” Emerging Infectious Disease 15(7):1068-1076 (2009).

Since inteins consistently interrupt highly conserved sites of intein-hosting proteins, splicing inhibition can cause a disruption of functions that are essential for the pathogen's survival. Inteins are therefore attractive targets for drug development. Green et al., “Spliceosomal Prp8 Intein at the Crossroads of Protein and RNA Splicing,” PLoS Biol 17(10):e3000104 (2019); Li et al., “Cisplatin Protects Mice From Challenge of Cryptococcus Neoformans by Targeting the Prp8 Intein,” Emerging Microbes & Infections 8(1):895-908 (2019); Paulus, H. “Protein Splicing Inhibitors as a New Class of Antimycobacterial Agents,” Drugs of the Future 32:973-984 (2007); Zhang et al., “Cisplatin Inhibits Protein Splicing, Suggesting Inteins as Therapeutic Targets in Mycobacteria,” The Journal of Bioligcal Chemistry 286(2):1277-1282 (2011); and Chan et al. “Exploring Intein Inhibition by Platinum Compounds as an Antimicrobial Strategy,” The Journal of Biological Chemistry 291(43):22661-22670 (2016). Additionally, inteins do not occur in multi-cellular organisms including humans nor in unicellular organisms including bacteria normally associated with the human gut flora, making intein-inhibiting drugs highly selective for intein-containing pathogens such as M. tuberculosis, C. neoformans, C. gattii, and A. fumigatus.

It was previously found that cisplatin, an FDA-approved chemotherapeutic drug, inhibited fungal Prp8 intein splicing in vitro and reduced fungal burden in vivo. Li et al., “Cisplatin Protects Mice From Challenge of Cryptococcus neoformans by Targeting the Prp8 Intein,” Emerging Microbes & Infections 8(1):895-908 (2019). The action of cisplatin is by the platinum ion being coordinated by the catalytic Cys1 of the intein, thereby inhibiting the first step of splicing and subsequent branched intermediate formation and extein ligation. However, the high cytotoxicity of cisplatin and its derivatives (Manohar, S. & Leung, N., “Cisplatin Nephrotoxicity: A Review of the Literature,” Journal of Nephrology 31(1):15-25 (2018); Barabas et al., “Cisplatin: A Review of Toxicities and Therapeutic Applications,” Veterinary and Comparative Oncology 6(1):1-18 (2008); and Paken et al., “Cisplatin-Associated Ototoxicity: A Review for the Health Professional,” Journal of Toxicology 2016:1809394 (2016)) may limit their use in immunocompromised patients. In the current study, a pilot screening of a small-molecule library was performed and a compound and its derivative that impede fungal Prp8 intein splicing in a dose-dependent manner was found. In addition, these molecules inhibited the Prp8 intein-containing fungi C. neoformans and C. gattii, but not the yeast Candida albicans, a major human pathogen that does not encode a Prp8 intein. Furthermore, C. neoformans treated with the small molecules led to accumulation of unspliced Prp8 precursor. The potency of these inhibitors is better than or comparable to the current frontline antifungal drugs. Mechanistic studies indicated that the small molecules inhibited Prp8 intein splicing by covalently binding to the Prp8 intein active-site residue Cys1, the nucleophile that initiates the protein splicing reaction.

The present disclosure is directed to overcoming these and other deficiencies in the art.

### SUMMARY

A first aspect of the present disclosure relates to a Prp8 intein splicing inhibitor of formula (I)

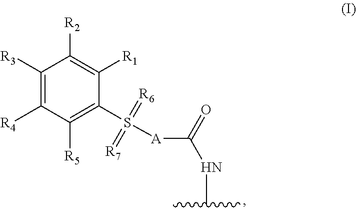

wherein R₁, R₂, R₃, R₄, and R₅ are independently selected from the group consisting of amine, amide, nitrogen oxide, C₁-C₂₃ alkyl, aryl, heteroaryl, carbocycle, heterocycle, oxygen, halogen, trifluoromethyl, and hydrogen, wherein the amine, amide, nitrogen oxide, C₁-C₂₃ alkyl, aryl, heteroaryl, carbocycle, heterocycle, oxygen, halogen, and trifluoromethyl can be optionally substituted with one or more halogen, hydrogen, C₁-C₃ alkyl, trifluoromethyl, or nitrogen oxide;

wherein R₆ and R₇ are independently selected from oxygen and hydrogen;

wherein A is independently selected from:

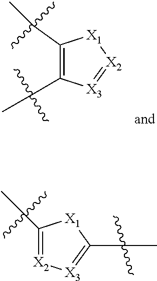

wherein

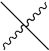

in A represent a point of attachment and wherein X₁, X₂, and X₃ are independently selected from carbon, nitrogen, sulfur, and oxygen; and

wherein [symbol] in formula (I) represents a point of attachment to at least one of:

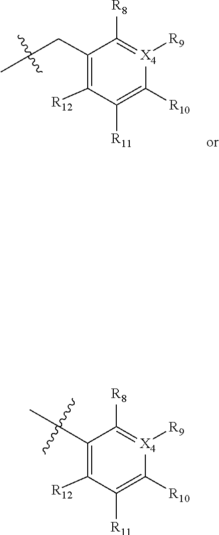

wherein X₄ is carbon or nitrogen;
wherein R₈, R₉, R₁₀, R₁₁, and R₁₂ are independently selected from hydrogen, halogen, trifluoromethyl, alkyl, and nitrogen oxide;
wherein if R₃ is chlorine, R₆ and R₇ are oxygen, R₈ is hydrogen, A is

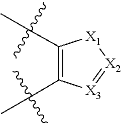

where X₂ is nitrogen, and X₄ is carbon, then (a) R₁₀ is not fluorine when R₈, R₉, R₁₁, and R₁₂ are hydrogen and (b) R₁₀ and R₁₂ are not fluorine when R₈, R₉, and R₁₁ are hydrogen.

A second aspect of the present disclosure relates to a method of treating and/or preventing a fungal infection. The method comprises administering a Prp8 intein splicing inhibitor of formula (I)

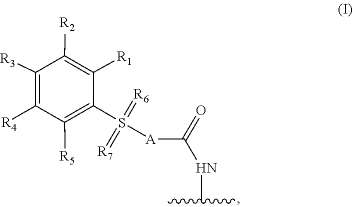

wherein R₁, R₂, R₃, R₄, and R₅ are independently selected from the group consisting of amine, amide, nitrogen oxide, C₁-C₂₃ alkyl, aryl, heteroaryl, carbocycle, heterocycle, oxygen, halogen, trifluoromethyl, and hydrogen, wherein the amine, amide, nitrogen oxide, C₁-C₂₃ alkyl, aryl, heteroaryl, carbocycle, heterocycle, oxygen, halogen, and trifluoromethyl can be optionally substituted with one or more halogen, hydrogen, C₁-C₃ alkyl, trifluoromethyl, or nitrogen oxide;

wherein R₆ and R₇ are independently selected from oxygen and hydrogen;

wherein A is independently selected from:

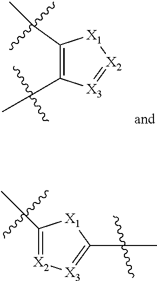

wherein

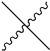

in A represent a point of attachment and wherein X₁, X₂, and X₃ are independently selected from carbon, nitrogen, sulfur, and oxygen; and

wherein [symbol] in formula (I) represents a point of attachment to at least one of:

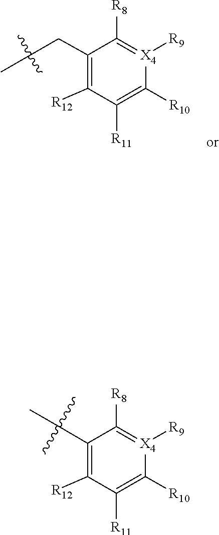

wherein Xₐ is carbon or nitrogen;
wherein R₈, R₉, R₁₀, R₁₁, and R₁₂ are independently selected from hydrogen, halogen, trifluoromethyl, alkyl, and nitrogen oxide, under conditions effective to treat and/or prevent a fungal infection.

A third aspect of the present disclosure relates to a method of inhibiting Prp8 intein expression or activity in a cell or tissue. The method comprises administering a compound of formula (I):

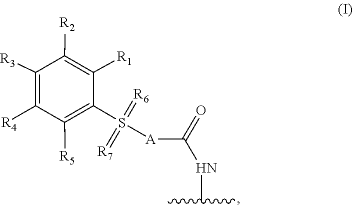

wherein R₁, R₂, R₃, R₄, and R₅ are independently selected from the group consisting of amine, amide, nitrogen oxide, C₁-C₂₃ alkyl, aryl, heteroaryl, carbocycle, heterocycle, oxygen, halogen, trifluoromethyl, and hydrogen, wherein the amine, amide, nitrogen oxide, C₁-C₂₃ alkyl, aryl, heteroaryl, carbocycle, heterocycle, oxygen, halogen, and trifluoromethyl can be optionally substituted with one or more halogen, hydrogen, C₁-C₃ alkyl, trifluoromethyl, or nitrogen oxide;

wherein R₆ and R₇ are independently selected from oxygen and hydrogen;

wherein A is independently selected from:

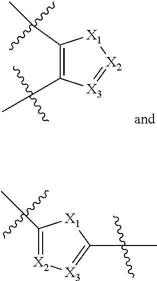

wherein

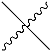

in A represent a point of attachment and wherein X₁, X₂, and X₃ are independently selected from carbon, nitrogen, sulfur, and oxygen; and

wherein [symbol] in formula (I) represents a point of attachment to at least one of:

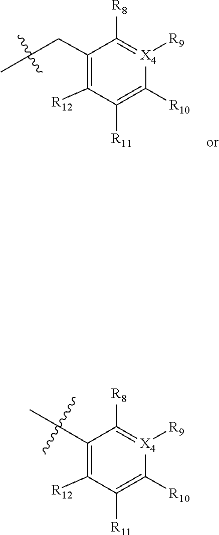

wherein X₄ is carbon or nitrogen;
wherein R₈, R₉, R₁₀, R₁₁, and R₁₂ are independently selected from hydrogen, halogen, trifluoromethyl, alkyl, and nitrogen oxide, under conditions effective to inhibit Prp8 intein expression or activity in a cell or tissue.

A fourth aspect of the present disclosure relates to a method for screening for compounds that inhibit Prp8 intein splicing comprising an assay. The assay comprises providing a GFP-Prp8 fusion protein; treating said GFP-Prp8 fusion protein with an intein splicing buffer; reacting the treated GFP-Prp8 fusion protein with one or more reagent; and detecting Prp8 intein splicing activity in a compound.

A fifth aspect of the present disclosure relates to a kit for predicting the likelihood of Prp8 inhibition. The kit comprises one or more agents that specifically recognize Prp8 intein expression or activity and a label that detects said recognition of Prp8 intein expression or activity by said one or more agents.

Self-splicing proteins, called inteins, are present in many human pathogens, including the emerging fungal threats Cryptococcus neoformans (Cne) and Cryptococcus gattii (Cga), the causative agents of cryptococcosis. Inhibition of protein splicing in Cryptococcus sp. interferes with activity of the only intein-containing protein, Prp8, an essential intron splicing factor. Here, a small molecule library was screened to find additional, potent inhibitors of the Cne Prp8 intein using a split-green fluorescence protein (GFP) splicing assay. This revealed the compound 6G-318S, with IC₅₀ values in the low micromolar range in the split-GFP assay and in a complementary split-luciferase system. A fluoride derivative of the compound 6G-318S displayed improved cytotoxicity in human lung carcinoma cells, although there was a slight reduction in splicing. 6G-318S and its derivative inhibited splicing of the Cne Prp8 intein in vivo in Escherichia coli and in C. neoformans. Moreover, the compounds repressed growth of wild-type C. neoformans and C. gattii. In contrast, the inhibitors were less potent at inhibiting growth of the inteinless Candida albicans. Drug resistance was observed when the Prp8 intein was overexpressed in C. neoformans, indicating specificity of this molecule towards the target. No off-target activity was observed, such as inhibition of serine/cysteine proteases. The inhibitors bound covalently to the Prp8 intein and binding was reduced when the active-site residue Cys1 was mutated. 6G-318S showed a synergistic effect with amphotericin B and additive to indifferent effects with a few other clinically used antimycotics. Overall, the identification of these small molecule intein splicing inhibitors opens up prospects for a new class of antifungals.

Cryptococcus neoformans is an opportunistic human pathogen, causing cryptococcal meningitis in immunocompromised individuals, with a mortality rate of more than 50%. It was previously found that the Prp8 intein was a viable drug target for C. neoformans. In an effort to find new therapies, screening assays were developed and a small molecule and its fluoride derivative were identified as potent Prp8 intein splicing inhibitors. These inhibitors bind covalently to an intein active-site residue and inhibit protein splicing, leading to non-functional Prp8, which is essential for fungal growth. These molecules inhibited growth of C. neoformans and showed synergistic or additive effects with FDA-approved antimycotics. Overall, the identification of these intein splicing inhibitors opens up prospects for a new class of antifungals.

### BRIEF DESCRIPTION OF THE DRAWINGS

FIGS. 1A-1B illustrate validation of the split-GFP Prp8 intein splicing assay. FIG. 1A shows the Prp8 intein splicing assay based on split-GFP. GFP (200 nM) and GFP-Prp8i (200 nM) were used with DMF or cisplatin (40 μM) for fluorescence detection. N=8. ***, P<0.001. FIG. 1B shows dose-response fitting of inhibition of splicing of the GFP-Prp8i by cisplatin. GFP-Prp8i (200 nM) was used. Cisplatin was in two-fold serial dilutions with concentrations ranging from 100 μM (30 μg/ml) to 0.78 μM (0.23 μg/ml). N=3.

FIGS. 2A-2F depict that pilot screening reveals a strong intein inhibitor. FIG. 2A shows chemical structures of hit compounds. FIG. 2B shows inhibition of split-GFP Prp8 splicing by hit compounds at 20 μM concentration. FIG. 2C shows inhibition of the split RLuc-Prp8 intein splicing by hit compounds at 20 μM concentration. FIGS. 2D and 2E show dose-dependent inhibition of the split GFP-Prp8i (200 nM) and RLuc-Prp8 (2 nM) intein splicing by 6G-318S, which was in two-fold serial dilutions with concentrations ranging from 100 μM to 0.78 μM. N=3. FIG. 2F shows dose-dependent inhibition of MIG-Prp8 splicing by 6G-318S. Left, SDS-PAGE analysis of each sample; right, relative percentage reduction of the MIG precursor upon treatment, compared to the starting material (T0). % P decrease is calculated as (% P_{T0}−% Pₛₐₘₚₗₑ)/(% P_{T0}−% P_{DMF})*100, where % P indicates percent of the MIG precursor at time point 0 (P_{T0}) and/or after ˜18 h of treatment with DMF (P_{DMF}) or cisplatin at different concentrations (P_{Sample}). N=3.

FIGS. 3A-3D show that analogs of 6G-318S have lower inhibitory activity but are intein-specific. FIG. 3A shows chemical structure of 6G-318S analogs. FIG. 3B shows inhibition of the split GFP-Prp8i splicing by the 6G-318S analogs at 20 μM. FIG. 3C shows dose-dependent inhibition of the GFP-Prp8i intein splicing by 6G-319S, which was in two-fold serial dilutions with concentrations ranging from 100 μM to 0.78 μM. N=3. FIG. 3D shows no inhibition of the trypsin and papain protease activities by intein inhibitors. Compound concentration for cisplatin, 6G-318S, and 6G-319S was set at 400 μM. Control inhibitor AP-2HCl was at 3.5 μM. N=3.

FIGS. 4A-4B depict improved cytotoxicity profile with 6G-319S. FIG. 4A shows cell viability assay with 6G-318S. FIG. 4B shows cell viability assay with 6G-319S. A549 cells were incubated with various concentrations of the drugs and then assayed for viability at 48 h post-incubation. Experimental data were fitted using a sigmoidal function. Cell viability with DMSO control was set as 100% viable. Medium alone with DMSO was set as 0% viability. N=3.

FIGS. 5A-5E show direct binding of 6G-318S and 6G-319S to the Prp8 intein. FIG. 5A shows PTSA for binding of compounds to the Prp8 intein. ΔTₘ was defined as T_{m-drug}-T_{m-DMSO}. FIG. 5B shows deconvolution of MS spectra of the recombinant wild-type Prp8 intein (pXI version) and its complex with 6G-318S and 6G-319S. FIG. 5C shows the proposed mechanism of inhibition of the Prp8 intein splicing by compounds 6G-318S and 6G-319S. Chemical reaction as illustrated. FIG. 5D shows deconvolution of MS spectra of the recombinant C1A Prp8 intein mutant (pXI version) and its complex with 6G-318S and 6G-319S. FIG. 5E shows kinetic binding data. SPR sensograms are shown with different colors for the binding of 6G-318S at different concentrations to recombinant Cga Prp8 or mutant inteins (pXI version) which were coupled to a ProteOn™ GLH sensor chip. Global fitting of data to a 1:1 binding model is shown in black.

FIGS. 6A-6E shows that 6G-318S inhibits Prp8 intein splicing in vivo. FIG. 6A shows that the antibody serum is specific to the Prp8 intein-containing fungi C. gattii and C. neoformans. Western blot (WB) analysis of cell lysates of C. gattii, C. neoformans, and C. albicans, using a polyclonal anti-Prp8 intein serum. FIG. 6B shows dose-dependent inhibition of Prp8 intein splicing in C. neoformans H99 by 6G-318S. FIG. 6C shows the time-course of inhibition of Prp8 intein splicing in C. neoformans H99 by 6G-318S. WB analysis of cell lysates of C. neoformans H99 treated with DMF or 6G-318S (0.16 ug/ml), using the polyclonal anti-Prp8 intein serum. FIG. 6D shows dose-dependent accumulation of high molecular band, likely corresponding to Prp8 precursor in 6G-318S-treated C. neoformans H99 cells. Left, WB analysis of cell lysates of C. neoformans H99 treated with DMF or 6G-318S at indicated concentrations, using the polyclonal anti-Prp8 intein serum; right, relative ratio between unspliced Prp8 protein precursor (P8P) and spliced intein (Int). N=3. FIG. 6E shows MS/MS spectra obtained from the fragmentation of Prp8 precursor, including both Prp8 intein and extein peptides. Fragment ions corresponding to y- and b-ions were observed (red or gray lines).

FIG. 7 illustrates efficacy of 6G318S in mouse models. BALB/c mice (5-6 wk old, male and female were dosed orally by either vehicle control (VC) or 6G318S-75 mg/kg once daily for 5 days. On day 5, all mice were euthanized, lungs were collected, weighed and homogenized in RPMI-1640-MOPS media. Serial 10-fold dilution of the homogenate is made and a volume of 100 ul is plated on to Sabouraud Dextrose-agar plates with antibiotics. Colonies were counted on day 3 and normalized to weight of the lung.

FIGS. 8A-8B show SDS-PAGE and GFP fluorescence analysis of GFP-Prp8 splicing. FIG. 8A shows coomassie blue and GFP fluorescence of GFP-Prp8 in the presence and absence of TCEP and 6G-118S (left panel). GFP-Prp8 (30 μM) was incubated with or without 1 mM TCEP and 6G-318S (50 μM) overnight (˜18 h). The gel was first visualized using GFP fluorescence before stained with Coomassie blue. Right panel, normalized band intensity (N=2). Band intensities for DMSO+TCEP for Coomassie blue and GFP were set as 100%. FIG. 8B shows GFP fluorescence analysis of SDS-PAGE samples of dose-dependent inhibition of GFP-Prp8 splicing by 6G-118S (upper panel). Lower panel, band intensities of spliced and ligated GFP product. GFP-Prp8 (30 μM) was incubated with or without 1 mM TCEP and 6G-318S overnight (˜18 h). Samples were run in SDS-PAGE. The gel was visualized using GFP fluorescence. MW, molecular weight. In SDS-PAGE, the PageRuler prestained protein ladder (Thermofisher, #26616) was used. The 70 KDa protein is slightly fluorescent under UV light.

FIG. 9 shows MFC determination of 6G-318S for C. neoformans strain H99. To determine MFC, serial 2-fold dilutions of the 6G118S (2.5 to 0.15 μg/ml) were made in 100 μl RPMI-1640 medium with MOPS in 96-well U-bottom plate. H99 cells (100 ul of 0.5×10⁵/ml) in log phase were added to each well. After incubating the cells at 30° C. for 48 h, the cell culture was visualized to determine MIC. Then, the cultured cells in selected wells were first suspended by gentle pipetting. 50 μl of the cell culture suspension from each well was plated onto the Sabouraud dextrose agar (SDA) plates and further incubated for 48 h. MFC is defined as the minimum concentration where there is no growth after plating to the SDA plates. MIC is defined as minimum concentration of the inhibitor required to result in no visible growth or ≥90% inhibition in absorbance at 630 nm as compared to no inhibitor control. The ratio of MFC/MIC≤54 is considered as fungicidal.

FIGS. 10A-10C show results of drug combination experiment and Effect of combination of 6G-318S with known antifungal drugs in inhibiting growth of C. neoformans (H99), using checkerboard assay. FIG. 10A shows combinatorial effects of 6G-318S with known antifungal drugs. Left, H99 cells treated with DMF; right, H99 cells treated with 6G-318S at 0.16 μg/ml. 2×10² H99 cells were inoculated into each well with precoated known antifungal drugs at increased concentrations. Pink color indicates fungal cell growth; light blue indicates no growth; mauve color in between blue and pink indicates the MIC breakpoint. Concentration series for all compounds except AmB were arranged horizontally, as indicated by horizontal triangle. AmB was arranged in the last column except for the last well (red or vertical box). FZ has an additional concentration in an extra well as indicated by the green box. Positive control was boxed black. FIG. 10B shows the Sensitire™ YeastOne™ plate layout (adapted from Thermofisher product flyer). Two-fold dilution series of drugs AND, MF, CAS, 5-FC, PZ, VOR, IZ, and FZ were arranged horizontally from column 1 to 11, whereas AmB (AB) was arranged vertically in column 12 all the way from A to G rows. FIG. 10C shows the effect of combination of 6G-318S with known antifungal drugs in inhibiting growth of C. neoformans (H99), using checkerboard assay. Plates were incubated for 48 h and cell densities were estimated by reading absorbance at 630 nm. Percentage inhibition compared to no drug control were calculated. MIC is defined as minimum concentration of the drug required to get 90% inhibition. FIC indices indicating if drug combinations are synergistic, additive or indifferent are presented in Table 2.

FIGS. 11A-11B shows that compound 6G-318S reacts with the Prp8 intein and covalently modifies the Prp8 active site Cys1 residue, regardless of the presence of extein. FIG. 11A shows MS/MS spectrum of modified peptide C[+221.0059]LQNGYR (SEQ ID NO: 1) from the Prp8 intein (pXI version) without extein. FIG. 11B shows MS/MS spectrum of modified peptide QASC[+221.0059]LQNGYR (SEQ ID NO: 2) from the Prp8 intein (pET28a version) with extra extein residues MGSSHHHHHHSSGLVPRGSHMASMTGGQQMGRGSEFELRRQAS (SEQ ID NO: 3) at the N-terminus and a Ser residue at the C-terminus. In both FIGS. 11A and 11B, the Prp8 inteins in complex with 6G-318S were trypsin-digested and subjected to LC-MS/MS analysis. Residue C was modified by a fragment of 6G-318S with a mass of 221.0059. Fragment ions corresponding to y- and b-ions were observed (red or gray lines).

FIG. 12 shows deconvoluted mass spectrometry of Cys-containing peptide modification by 6G-318S. A Cys-containing peptide (EVPANSTVLSFCAFAVDAAKAYKDYLAS) (SEQ ID NO: 4) was synthesized by ABclonal Science (Woburn, Mass.). The purity and mass of the peptide was confirmed by liquid chromatography and mass spectrometry (LC-MS). To assess the reactivity of the peptide with 6G-318S, an Agilent 6530B Q-TOF system configured with Agilent 1260 UPLC was used. Briefly, peptide (30 μM) was incubated with DMSO (black), 6G-318S (300 μM) (red) or 6G-318S (300 μM) and 100 mM KCl (green) in PBS buffer in the presence of 3 mM TCEP for 18 hr at 22° C. The resulting mixture was diluted 20-fold with 0.01% formic acid prior to a LC-MS analysis. Separation of the compound mixture was achieved on a Discovery BIO wide pore C5 column (3 μm, 2.1×50 mm), using a gradient from solvent A (95% water, 5% acetonitrile, 0.1% formic acid) to solvent B (100% acetonitrile, 0.1% formic acid). Injections were made in 20% B, which was held for 1 min, then ramped to 80% B in 3 min. The mobile phase was held at 80% B for 2 min, returned to starting conditions over 0.1 min, and allowed to re-equilibrate for 6.9 min. Flow rate was constant at 400 μL/min for the duration of the run. The column was held at 45° C. Column eluent was infused into an Agilent 6530B Q-TOF mass spectrometer configured with a dual electrospray source. Data were collected in positive ion mode, scanning from 700-2000 m/z at a rate of 1 spectrum per second. The parameters for the operation were as follows: gas temperature, 350° C.; gas flow, 13 L/min; nebulizer, 35 psi; vcap, 4,000 V; fragmentor, 175 V; skimmer 1, 65 V. Raw data files were processed using an Agilent MassHunter Qualitative Analysis software B7.0.

### DETAILED DESCRIPTION

A first aspect of the present disclosure relates to a Prp8 intein splicing inhibitor of formula (I)

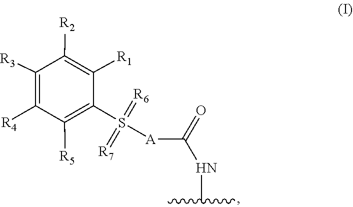

wherein R₁, R₂, R₃, R₄, and R₅ are independently selected from the group consisting of amine, amide, nitrogen oxide, C₁-C₂₃ alkyl, aryl, heteroaryl, carbocycle, heterocycle, oxygen, halogen, trifluoromethyl, and hydrogen, wherein the amine, amide, nitrogen oxide, C₁-C₂₃ alkyl, aryl, heteroaryl, carbocycle, heterocycle, oxygen, halogen, and trifluoromethyl can be optionally substituted with one or more halogen, hydrogen, C₁-C₃ alkyl, trifluoromethyl, or nitrogen oxide;

wherein R₆ and R₇ are independently selected from oxygen and hydrogen;

wherein A is independently selected from:

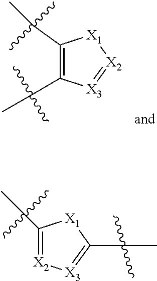

wherein in

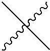

A represent a point of attachment and wherein X₁, X₂, and X₃ are independently selected from carbon, nitrogen, sulfur, and oxygen; and

wherein [symbol] in formula (I) represents a point of attachment to at least one of:

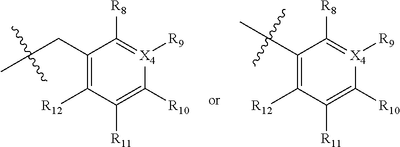

wherein X₄ is carbon or nitrogen;
wherein R₈, R₉, R₁₀, R₁₁, and R₁₂ are independently selected from hydrogen, halogen, trifluoromethyl, alkyl, and nitrogen oxide;
wherein if R₃ is chlorine, R₆ and R₇ are oxygen, R₈ is hydrogen, A is

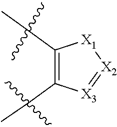

where X₂ is nitrogen, and X₄ is carbon, then (a) R₁₀ is not fluorine when R₈, R₉, R₁₁, and R₁₂ are hydrogen and (b) R₁₀ and R₁₂ are not fluorine when R₈, R₉, and R₁₁ are hydrogen.

It is to be appreciated that certain aspects, modes, embodiments, variations, and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this disclosure belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±1 or ±10%, or any point therein, and remain within the scope of the disclosed embodiments.

Where a range of values is described, it should be understood that intervening values, unless the context clearly dictates otherwise, between the upper and lower limit of that range and any other stated or intervening value in other stated ranges, may be used in the embodiments described herein.

As used herein, the terms “subject,” “individual” or “patient,” used interchangeably, means any animal, including mammals, such as mice, rats, other rodents, rabbits, dogs, cats, swine, cattle, sheep, horses, or primates, such as humans.

As used herein, the term “purified” means that when isolated, the isolate contains at least 90%, at least 95%, at least 98%, or at least 99% of a compound described herein by weight of the isolate.

As used herein, the phrase “substantially isolated” means a compound that is at least partially or substantially separated from the environment in which it is formed or detected.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

As used herein, any “R” group(s) represents substituents that may be attached to an indicated atom. An R group may be substituted or unsubstituted. If two R groups are described as “together with the atoms to which they are attached” forming a ring or ring system, it means that the collective unit of the atoms, intervening bonds and the two R groups are the recited ring.

C₁ to C₂₃ hydrocarbon includes alkyl, cycloalkyl, polycycloalkyl, alkenyl, alkynyl, aryl, and combinations thereof. Examples include benzyl, phenethyl, propargyl, allyl, cyclohexylmethyl, adamantyl, camphoryl, and naphthylethyl. Hydrocarbon refers to any substituent included of hydrogen and carbon as the only elemental constituents.

The term “alkyl” includes an aliphatic hydrocarbon group which may be straight or branched having about 1 to about 23 carbon atoms in the chain. For example, straight or branched carbon chain could have 1 to 10 carbon atoms or 1 to 6 carbon atoms. Branched means that one or more lower alkyl groups such as methyl, ethyl, or propyl are attached to a linear alkyl chain. Alkyl includes a hydrocarbon that is fully saturated (i.e., contains no double or triple bonds) and combinations thereof. (e.g., 1 to 10 carbon atoms, such as 1 to 6 carbon atoms). Examples of alkyl groups include but are not limited to methyl, ethyl, propyl, n-propyl, isopropyl, butyl, isobutyl, n-butyl, s-butyl, t-butyl, n-pentyl, and 3-pentyl. An alkyl group may have between 1 to about 23 carbon atoms (whenever it appears herein, a numerical range such as “1 to 23” refers to each integer in the given range; e.g., “1 to 23 carbon atoms” means that the alkyl group may consist of 1 carbon atom, 2 carbon atoms, 3 carbon atoms, 4 carbon atoms, 5 carbon atoms, etc., and up to and including 23 carbon atoms, although the present disclosure also covers the occurrence of the term “alkyl” where no numerical range is designated). For example, “C₁-C₆ alkyl” indicates that there are between one and six carbon atoms in the alkyl chain (i.e., the alkyl chain is selected from the group consisting of methyl, ethyl, propyl, iso-propyl, n-butyl, iso-butyl, sec-butyl, and t-butyl).

As described herein, “alkenyl” refers to a straight or branched hydrocarbon chain containing one or more double bonds. An alkenyl group may have about 2 to about 23 carbon atoms, although the present description also covers the occurrence of the term “alkenyl” where no numerical range is designated. The alkenyl group may also be a medium size alkenyl having 2 to 9 carbon atoms. The alkenyl group could also be a lower alkenyl having between 2 and 6 carbon atoms. For example, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups may include, but are not limited to, ethenyl, propenyl, butenyl, pentenyl, and hexenyl.

As described herein, “alkynyl” includes a straight or branched hydrocarbon chain containing one or more triple bonds. An alkynyl group may have between about 2 and about 23 carbon atoms, although the present description also includes the occurrence of the term “alkynyl” where no numerical range is designated. As an example, “C₂-C₆ alkynyl” indicates between two and six carbon atoms in the alkynyl chain (i.e., the alkynyl chain may be selected from the group consisting of ethynyl, propyn-1-yl, propyn-2-yl, butyn-1-yl, butyn-3-yl, butyn-4-yl, and 2-butynyl). Typical alkynyl groups may include, but are not limited to, ethynyl, propynyl, butynyl, pentynyl, and hexynyl, and the like.

As described herein, “heteroalkyl” may include a straight or branched hydrocarbon chain containing one or more heteroatoms, that is, an element other than carbon, including but not limited to, nitrogen, oxygen, and sulfur, in the chain backbone. A heteroalkyl group may have between 1 and 20 carbon atoms, although the present disclosure also includes the occurrence of the term “heteroalkyl” where no numerical range is designated. For example, “C₄-C₆ heteroalkyl” may indicate that there are between four and six carbon atoms in the heteroalkyl chain and additionally one or more heteroatoms in the backbone of the chain.

Aromatic as described herein refers to a ring or ring system having a conjugated pi electron system and includes both carbocyclic aromatic (e.g., phenyl) and heterocyclic aromatic groups (e.g., pyridine). Aromatics may include monocyclic or fused-ring polycyclic (i.e., rings which share adjacent pairs of atoms) groups provided the entire ring system is aromatic.

“Aryl” as described herein includes an aromatic ring or ring system (e.g., two or more fused rings that share two adjacent carbon atoms) containing only carbon in the ring backbone. The present disclosure also includes the occurrence of the term “aryl” where no numerical range is designated. In one embodiment, the aryl group has between 6 and 10 carbon atoms. An aryl group may be designated as “C₆-C₁₀ aryl” for example. Representative aryl groups include, but are not limited to, phenyl, naphthyl, azulenyl, and anthracenyl.

An “aralkyl” or “arylalkyl” as described herein may include an aryl group connected, as a substituent, via an alkylene group, such as for example C₇-C₁₄ aralkyl and the like, including but not limited to benzyl, 2-phenylethyl, 3-phenylpropyl, and naphthylalkyl.

The term “heteroaryl” includes an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “heteroaryl.” The heteroaryl group may have between 5-18 ring members (i.e., the number of atoms making up the ring backbone, including carbon atoms and heteroatoms), although the present disclosure also includes the occurrence of the term “heteroaryl” where no numerical range is designated. Preferred heteroaryls contain between about 5 to 10 ring atoms, or between about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include thienyl, phthalazinyl, pyridinyl, benzoxazolyl, benzothienyl, pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-15 a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

A “heteroaralkyl” or “heteroarylalkyl” refers to a heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl.

Unless otherwise specified, the term “carbocycle” is intended to include ring systems in which the ring atoms are all carbon but of any oxidation state. When the carbocyclyl is a ring system, two or more rings may be joined together in a fused, bridged, or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, and the present use of the term “carbocyclyl” also includes when no numerical range is designated. Thus (C₃-C₁₂) carbocycle, for example, refers to both non-aromatic and aromatic systems, including such systems as cyclopropane, benzene, and cyclohexene. Carbocycle, if not otherwise limited, refers to monocycles, bicycles, and polycycles.

As used herein, “cycloalkyl” means a fully saturated carbocyclyl ring or ring system. Cycloalkyl is a subset of hydrocarbon and includes cyclic hydrocarbon groups of from 3 to 8 carbon atoms. Examples of cycloalkyl groups include c-propyl, c-butyl, c-pentyl, and norbornyl (e.g., cyclopropyl, cyclobutyl, cyclopentyl, and cyclohexyl).

As used herein, the term “C₁-C₂₃” includes C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, and C₂₃, and a range defined by any of the two numbers. For example, C₁-C₂₃ alkyl includes C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, and C₂₃ alkyl, C₁-C₂₃ alkyl, C₁-C₂₀ alkyl, C₁-C₁₅ alkyl, C₁-C₁₀ alkyl, C₁-C₆ alkyl, etc. Similarly, C₂-C₂₃ alkenyl includes C₂, C₃, C₄, C₅, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, and C₂₃ alkenyl, C₂-C₂₃ alkenyl, C₂-C₂₀ alkenyl, C₂-C₁₅ alkenyl, C₂-C₁₀ alkenyl, C₂-C₆ alkenyl, etc.; and C₂-C₂₃ alkynyl includes C₂, C₃, C₄, C₅, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, and C₂₃ alkynyl, C₂-C₂₃ alkynyl, C₂-C₂₀ alkynyl, C₂-C₁s alkynyl, C₂-C₁₀ alkynyl, C₂-C₆ alkynyl, etc. C₃-C₅ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl. As used herein, the term “C₁-C₆” includes C₁, C₂, C₃, C₄, C₅, and C₆, and a range defined by any of the two numbers C₁-C₆.

As used herein, “heterocyclyl” or “heterocycle” refers to a stable 3- to 18-membered ring (radical) which consists of carbon atoms and from one to five heteroatoms selected from the group consisting of nitrogen, oxygen, and sulfur. For purposes of this disclosure, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the occurrence of the term “heterocyclyl” where no numerical range is designated is included. Examples of such heterocycles include, without limitation, acridinyl, carbazolyl, imidazolinyl, oxepanyl, thiepanyl, dioxopiperazinyl, pyrrolidonyl, pyrrolidionyl, oxiranyl, azepinyl, azocanyl, pyranyl dioxolanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, thiamorpholinyl sulfone, and tetrahydroquinoline. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “halogen” or “halo” as used herein, may include any one of the radio-stable atoms of column 7 of the Periodic Table of the Elements, e.g., fluorine, chlorine, bromine, or iodine. In one embodiment, halogen may be a fluorine or chlorine atom.

Basic addition salts can be prepared by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term amide, as described herein, refers to non-toxic amides derived from ammonia, primary C₁-to-C₆ alkyl amines and secondary C₁-to-C₆ dialkyl amines. In the case of secondary amines, the amine can also be in the form of a 5- or 6-membered heterocycle containing one nitrogen atom. Amides derived from ammonia, C₁-to-C₃ alkyl primary amides and C₁-to-C₂ dialkyl secondary amides are preferred. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

As described herein, the term nitrogen oxide includes nitrogen- and oxide-containing compounds, for example, NO₂, NO, N₂O, and N₂O₅. The term “nitro” as used interchangeably herein to describe nitrogen- and oxide-containing compounds, may include, for example, a —NO₂ group.

The term “substituted” or “substitution” of an atom means that one or more hydrogen on the designated atom is replaced with a selection from the indicated group, provided that the designated atom's normal valency is not exceeded. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents. Wherever a group is described as “optionally substituted” that group may be substituted with the above substituents.

“Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture.

The compounds described herein may contain asymmetric centers and may thus give rise to enantiomers, diastereomers, and other stereoisomeric forms which may be defined in terms of absolute stereochemistry as (R)- or (S)-. The present invention is meant to include all such possible diastereomers as well as their racemic and optically pure forms. Optically active (R)- and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (E)- and (Z)-geometric isomers. Likewise, all tautomeric forms are intended to be included.

The compounds described herein may possess one or more centers of chirality. Various examples of each of such compounds may differ from one another by nature of their stereochemistry at one or more chiral center. A given compound may therefore exist in a stereochemically pure state, consisting of a single stereoisomer, or include a racemic mixture of different enantiomers that possess different stereochemistry at one or more chiral centers from one another. Notwithstanding any chemical identities disclosed herein, also included within the present disclosure are compounds which may include a racemic mixture of various stereoisomers of the compounds described herein or may include isolates of a given stereoisomer of a given compound, identical to or different from any particular stereoisomer specifically identified herein.

Enantiomerically pure means greater than 80 e.e., and preferably greater than 90 e.e. For the purpose of the present disclosure, a “pure” or “substantially pure” stereoisomer is intended to mean that the stereoisomer is at least 95% of the configuration shown and 5% or less of other stereoisomers, or at least 97% of the configuration shown and 3% or less of other stereoisomers, or at least 99% of the configuration shown and 1% or less of other stereoisomers.

In one embodiment, in the Prp8 intein splicing inhibitor of formula (I), one or more of R₁, R₂, R₃, R₄, and R₅ is hydrogen, halogen, or trifluoromethyl. In another embodiment, in the Prp8 intein splicing inhibitor of formula (I), one or more of R₁, R₂, R₃, R₄, and R₅ is chlorine. In one embodiment, in the Prp8 intein splicing inhibitor of formula (I), one or more of X₁, X₂, and X₃ is N. In one embodiment, in the Prp8 intein splicing inhibitor of formula (I), one or more of X₁, X₂, and X₃ is S. In one embodiment, in the Prp8 intein splicing inhibitor of formula (I), one or more of R₈, R₉, R₁₀, R₁₁, and R₁₂ is hydrogen, fluorine, chlorine, bromine, trifluoromethyl, methyl, or nitrogen oxide.

In one embodiment, in the Prp8 intein splicing inhibitor of formula (I), the Prp8 intein splicing inhibitor has the formulae:

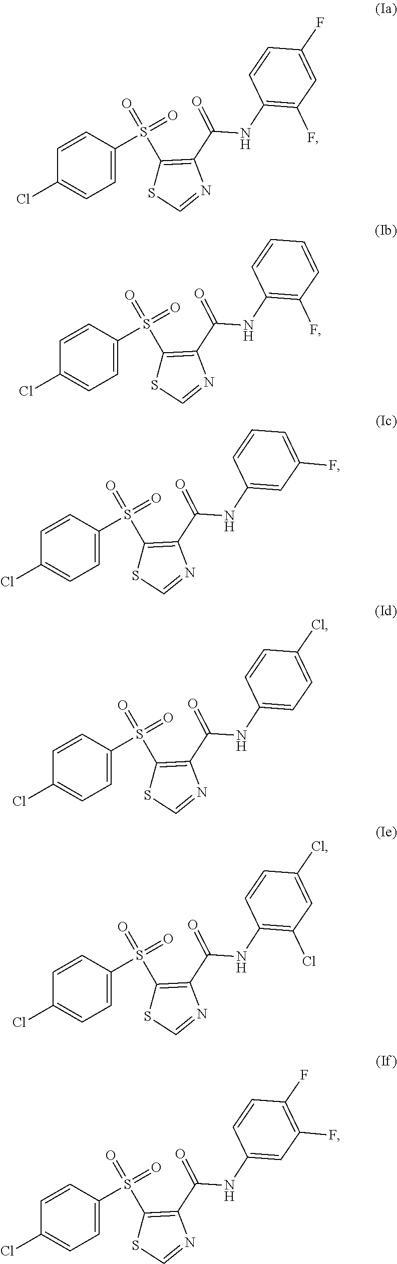 [SMILES: O=C(Nc1ccc(F)cc1F)c1ncsc1S(=O)(=O)c1ccc(Cl)cc1 ; O=C(Nc1ccccc1F)c1ncsc1S(=O)(=O)c1ccc(Cl)cc1 ; O=C(Nc1cccc(F)c1)c1ncsc1S(=O)(=O)c1ccc(Cl)cc1 ; O=C(Nc1ccc(Cl)cc1)c1ncsc1S(=O)(=O)c1ccc(Cl)cc1 ; O=C(Nc1ccc(Cl)cc1Cl)c1ncsc1S(=O)(=O)c1ccc(Cl)cc1 ; O=C(Nc1ccc(F)c(F)c1)c1ncsc1S(=O)(=O)c1ccc(Cl)cc1]

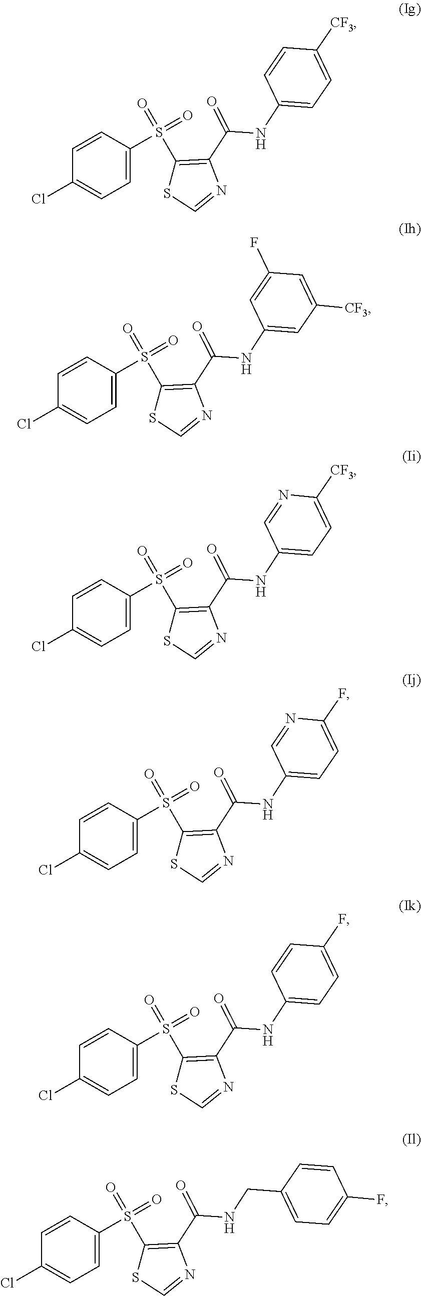 [SMILES: O=C(Nc1ccc(C(F)(F)F)cc1)c1ncsc1S(=O)(=O)c1ccc(Cl)cc1 ; O=C(Nc1cc(F)cc(C(F)(F)F)c1)c1ncsc1S(=O)(=O)c1ccc(Cl)cc1 ; O=C(Nc1ccc(C(F)(F)F)nc1)c1ncsc1S(=O)(=O)c1ccc(Cl)cc1 ; O=C(Nc1ccc(F)nc1)c1ncsc1S(=O)(=O)c1ccc(Cl)cc1 ; O=C(Nc1ccc(F)cc1)c1ncsc1S(=O)(=O)c1ccc(Cl)cc1 ; O=C(NCc1ccc(F)cc1)c1ncsc1S(=O)(=O)c1ccc(Cl)cc1]

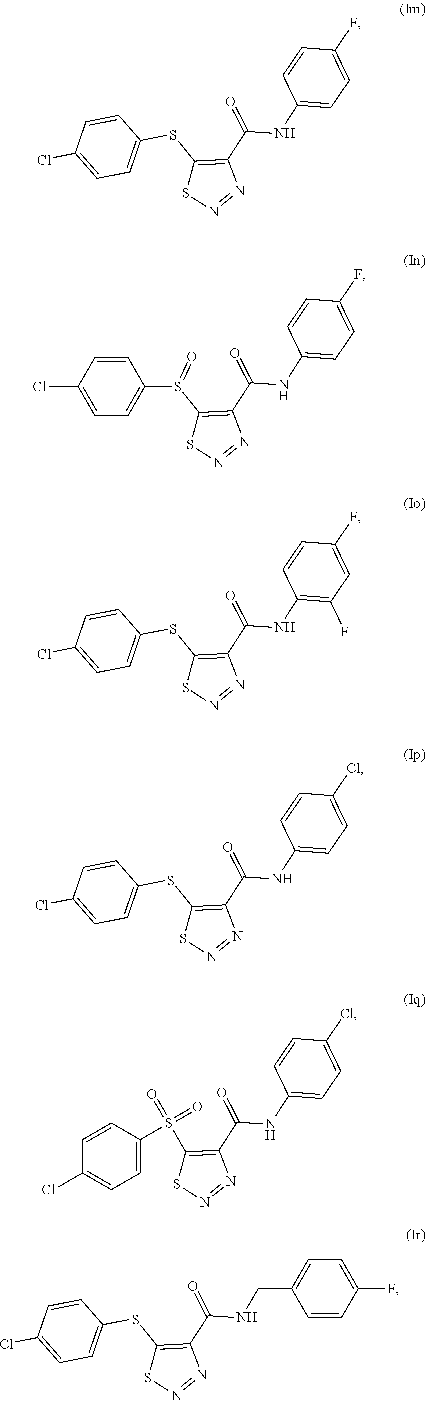 [SMILES: O=C(Nc1ccc(F)cc1)c1nnsc1Sc1ccc(Cl)cc1 ; O=C(Nc1ccc(F)cc1)c1nnsc1S(=O)c1ccc(Cl)cc1 ; O=C(Nc1ccc(F)cc1F)c1nnsc1Sc1ccc(Cl)cc1 ; O=C(Nc1ccc(Cl)cc1)c1nnsc1Sc1ccc(Cl)cc1 ; O=C(Nc1ccc(Cl)cc1)c1nnsc1S(=O)(=O)c1ccc(Cl)cc1 ; O=C(NCc1ccc(F)cc1)c1nnsc1Sc1ccc(Cl)cc1]

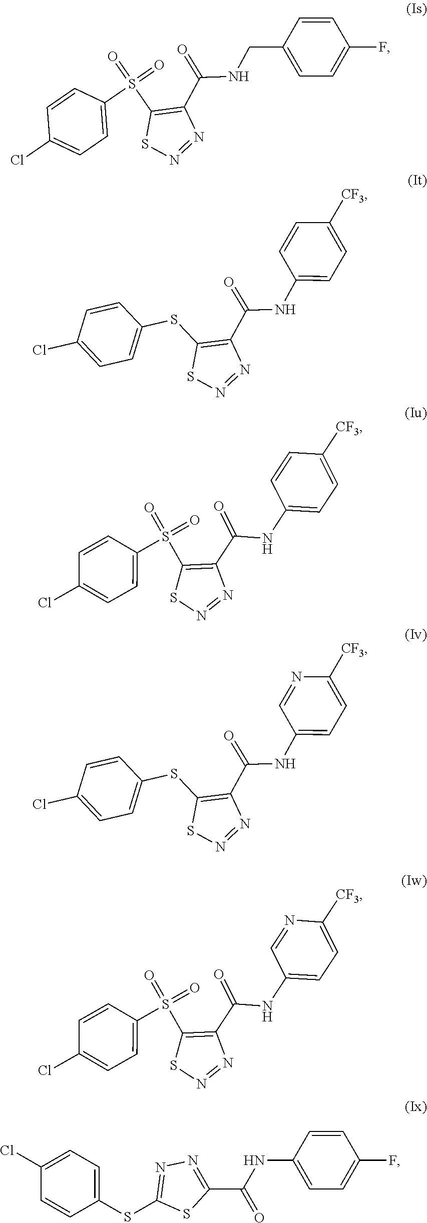 [SMILES: O=C(NCc1ccc(F)cc1)c1nnsc1S(=O)(=O)c1ccc(Cl)cc1 ; O=C(Nc1ccc(C(F)(F)F)cc1)c1nnsc1Sc1ccc(Cl)cc1 ; O=C(Nc1ccc(C(F)(F)F)cc1)c1nnsc1S(=O)(=O)c1ccc(Cl)cc1 ; O=C(Nc1ccc(C(F)(F)F)nc1)c1nnsc1Sc1ccc(Cl)cc1 ; O=C(Nc1ccc(C(F)(F)F)nc1)c1nnsc1S(=O)(=O)c1ccc(Cl)cc1 ; O=C(Nc1ccc(F)cc1)c1nnc(Sc2ccc(Cl)cc2)s1]

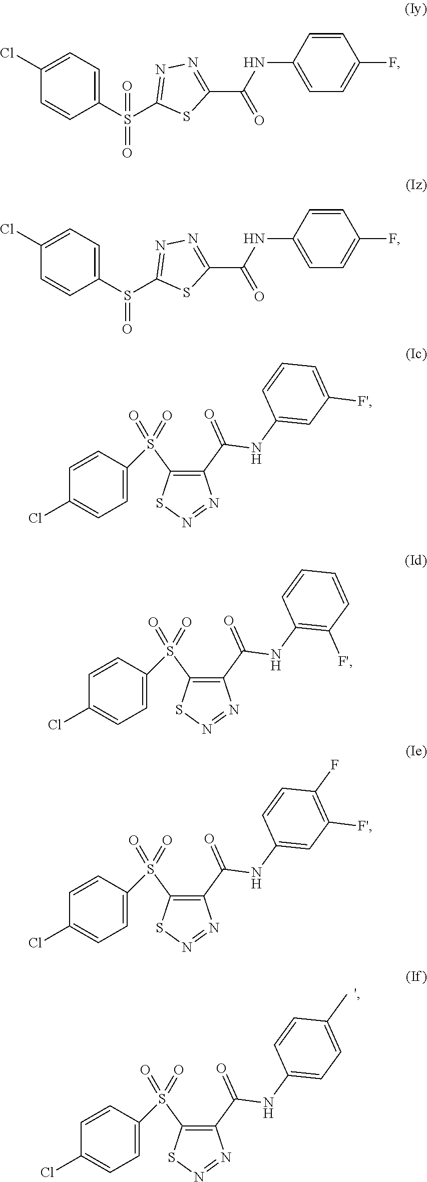

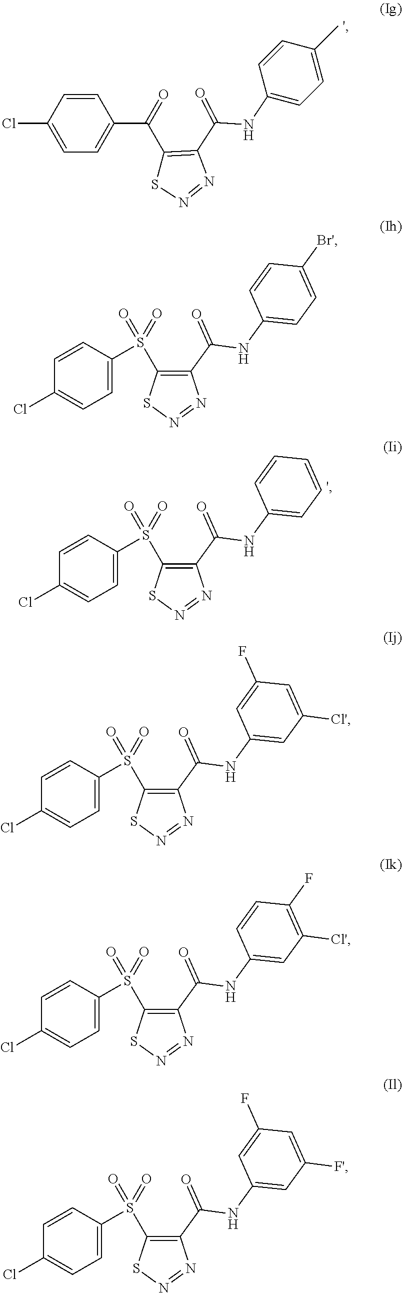

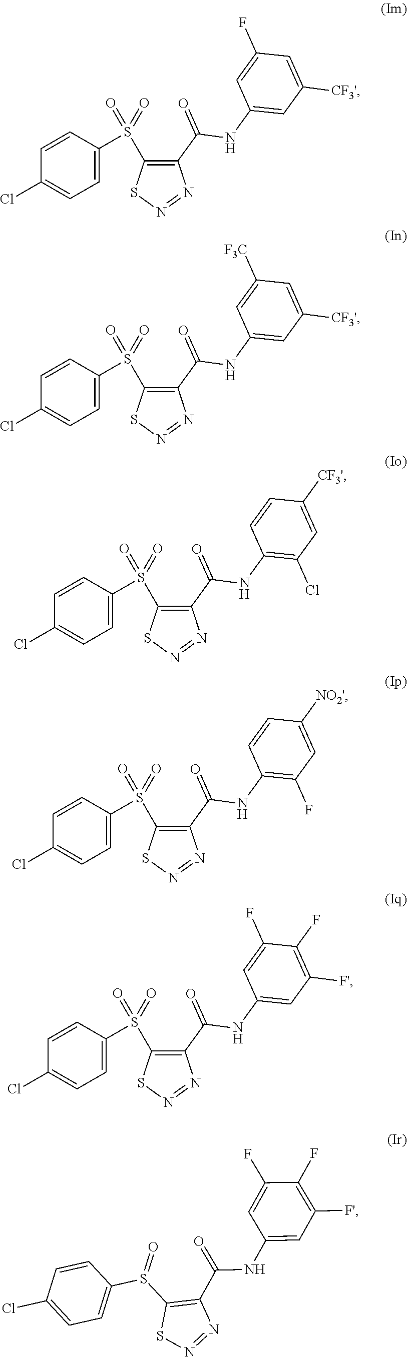

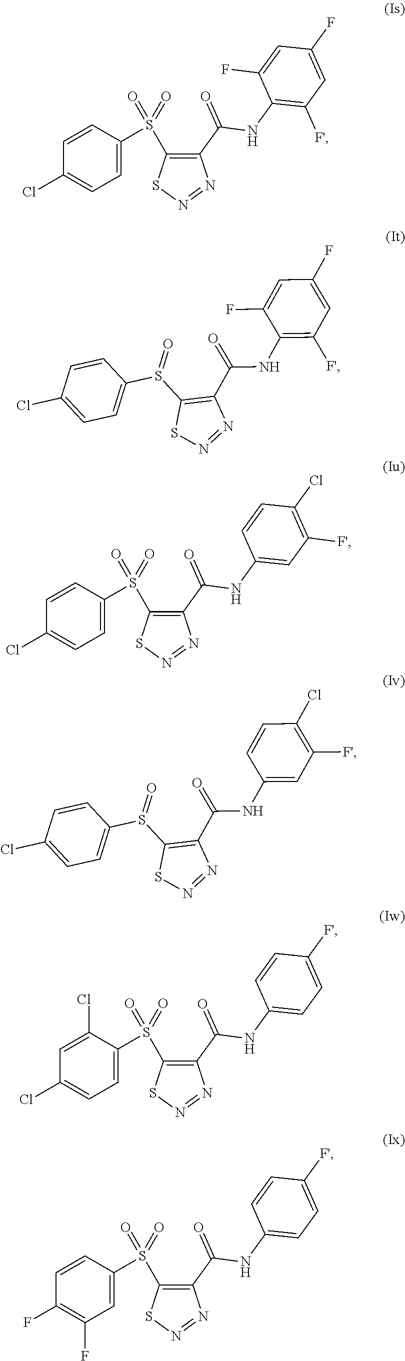

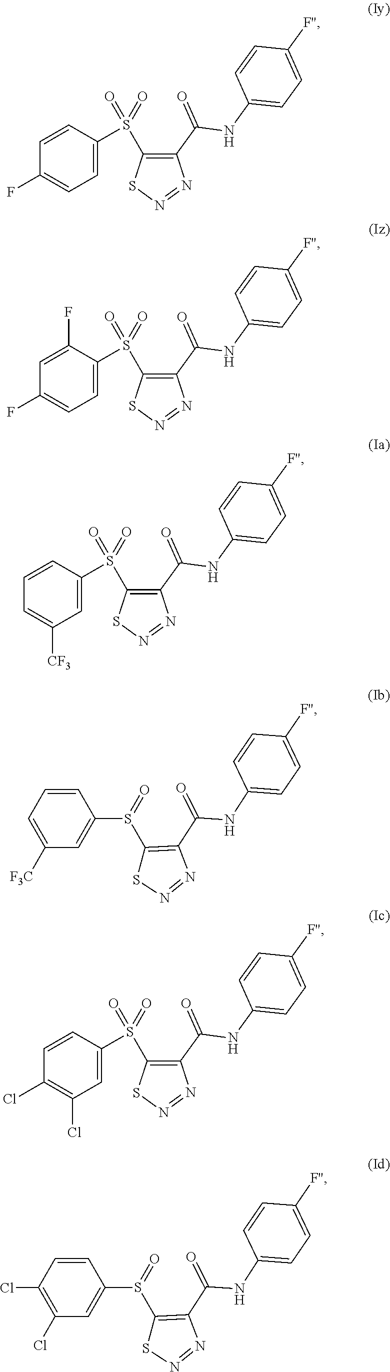

or a pharmaceutically acceptable salt thereof.

Also provided herein is a pharmaceutical composition comprising a compound disclosed herein, or a pharmaceutically acceptable salt form thereof, and a pharmaceutically acceptable carrier, diluent, or excipient.

While it may be possible for the compounds disclosed herein to be administered as the raw chemical, it is preferable to present them as a pharmaceutical composition. According to a further aspect, the present invention provides a pharmaceutical composition comprising a compound of formula (I) (e.g., any compound of formulae (Ia) through (Id″)) or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

In one embodiment, the composition used herein further comprises a pharmaceutically acceptable carrier. “Pharmaceutically acceptable carriers” as used herein refer to conventional pharmaceutically acceptable carriers. See Remington's Pharmaceutical Sciences, by E. W. Martin, Mack Publishing Co., Easton, Pa., 15th Edition (1975), which is hereby incorporated by reference in its entirety, and describes compositions suitable for pharmaceutical delivery of the compositions described herein. In particular, a pharmaceutically acceptable carrier as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits. In one embodiment, the pharmaceutically acceptable carrier is selected from the group consisting of a liquid filler, a solid filler, a diluent, an excipient, a solvent, and an encapsulating material.

Pharmaceutically acceptable carriers (e.g., additives such as diluents, immunostimulants, adjuvants, antioxidants, preservatives, and solubilizing agents) are non-toxic to the cell or subject being exposed thereto at the dosages and concentrations employed. Examples of pharmaceutically acceptable carriers include water, e.g., buffered with phosphate, citrate, and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; adjuvants (selected so as to avoid adjuvant-induced toxicity, such as a (3-glucan as described in U.S. Pat. No. 6,355,625, which is hereby incorporated by reference in its entirety, or a granulocyte colony stimulating factor (GCSF)); hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

In one embodiment, the compound described herein may further comprise an adjuvant. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles.

Pharmaceutically acceptable carriers refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid, and the like. It can also be desirable to include isotonic agents such as sugars, sodium chloride, and the like. Prolonged absorption of the injectable pharmaceutical form can be brought about by the inclusion of agents, such as aluminum monostearate and gelatin, which delay absorption. Injectable depot forms may be formed by microencapsule matrices of the compound in biodegradable polymers such as polylactide-polyglycolide, poly(orthoesters), and poly(anhydrides). Depending upon the ratio of compound to polymer and the nature of the particular polymer employed, the rate of compound release can be controlled. Depot injectable formulations are also prepared by entrapping the compound in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The compounds described herein may comprise different types of carriers depending on whether it is to be administered in solid, liquid, or aerosol form, and whether it needs to be sterile for such routes of administration as injection. The compounds described herein may be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, e.g., Remington's Pharmaceutical Sciences, 18th Ed. Mack Printing Company, 1990, which is hereby incorporated by reference in its entirety).

The phrase “pharmaceutically acceptable salt” refers to salts prepared from pharmaceutically acceptable non-toxic acids or bases including inorganic acids and bases and organic acids and bases. When the compounds described herein are basic, salts may be prepared from pharmaceutically acceptable non-toxic acids including inorganic and organic acids. Suitable pharmaceutically acceptable acid addition salts for the compounds described herein include acetic, adipic, alginic, ascorbic, aspartic, benzenesulfonic (besylate), benzoic, betulinic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, ursolic, and the like. When the compounds contain an acidic side chain, suitable pharmaceutically acceptable base addition salts for the compounds described herein include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine), and procaine. Further pharmaceutically acceptable salts include, when appropriate, non-toxic ammonium cations and carboxylate, sulfonate and phosphonate anions attached to alkyl having from 1 to 20 carbon atoms.

The compounds described herein may be formulated into a composition in a free base, neutral or salt form. Pharmaceutically acceptable salts, include the acid addition salts, for example, those formed with the free amino groups of a proteinaceous composition, or those formed with inorganic acids such as, for example, hydrochloric or phosphoric acids, or such organic acids as acetic, oxalic, tartaric, or mandelic acid. Salts formed with the free carboxyl groups can also be derived from inorganic bases such as for example, sodium, potassium, ammonium, calcium, or ferric hydroxides; or such organic bases as isopropylamine, trimethylamine, histidine, or procaine. Upon formulation, solutions may be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules, and the like.

As used herein, the term “physiologically functional derivative” refers to any pharmaceutically acceptable derivative of a compound described herein, that, upon administration to a mammal, is capable of providing (directly or indirectly) a compound of the present disclosure or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is hereby incorporated by reference in its entirety.

As used herein, the term “effective amount” includes an amount of a compound or pharmaceutical agent that will elicit the biological or medical response of a cell, tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of the compounds of the present disclosure, as well as salts, solvates, and physiological functional derivatives thereof, may be administered as the raw chemical. Additionally, the active ingredient may be presented as a pharmaceutical composition.

Pharmaceutical compositions of the present disclosure comprise an effective amount of one or more compound of formula (I) (e.g., any compound of formulae (Ia) through (Id″)), or additional agent dissolved or dispersed in a pharmaceutically acceptable carrier. The phrases “pharmaceutical or pharmacologically acceptable” refer to molecular entities and compositions that do not produce an adverse, allergic, or other untoward reaction when administered to an animal, such as, for example, a human, as appropriate. See Remington's Pharmaceutical Sciences, 18th Ed. Mack Printing Company, 1990, which is hereby incorporated by reference in its entirety. Moreover, for animal (e.g., human) administration, it will be understood that preparations should meet sterility, pyrogenicity, general safety and purity standards as required by FDA Office of Biological Standards.

Further in accordance with the present disclosure, the compositions described herein that are suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of the composition contained therein, its use in administrable composition for use in practicing the present disclosure is appropriate. Examples of carriers or diluents include fats, oils, water, saline solutions, lipids, liposomes, resins, binders, fillers, and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

In accordance with the present disclosure, the compounds described herein may be combined with the carrier in any convenient and practical manner, i.e., by solution, suspension, emulsification, admixture, encapsulation, absorption and the like. Such procedures are routine for those skilled in the art.

In one embodiment of the present disclosure, the composition described herein is combined or mixed thoroughly with a semi-solid or solid carrier. The mixing can be carried out in any convenient manner such as grinding. Stabilizing agents may also be added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, and mannitol.

In further embodiments, the present disclosure may include the use of pharmaceutical lipid vehicle compositions that include the compounds described herein, one or more lipids, and an aqueous solvent. As used herein, the term “lipid” may include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or man-made). A lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Compounds other than those specifically described herein that are understood by one of skill in the art as lipids are also encompassed by the compositions the present disclosure.

One of ordinary skill in the art would be familiar with the range of techniques that can be employed for dispersing a composition in a lipid vehicle. For example, the compounds described herein may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

In one embodiment of the present disclosure, the compounds are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tablets, troches, capsules, elixirs, suspensions, syrups, wafers, and the like. Tablets, troches, pills, capsules and the like may also contain the following: a binder, such as, for example, gum tragacanth, acacia, cornstarch, gelatin or combinations thereof, an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, and/or orange flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin, and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

In further embodiments, the compound may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy injectability exists. It must be stable under the conditions of manufacture and storage and must be preserved against the contaminating action of microorganisms, such as bacteria and fungi.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., “Remington's Pharmaceutical Sciences” 15^{th} Edition, pages 1035-1038 and 1570-1580, which is hereby incorporated by reference in its entirety). Some variation in dosage will necessarily occur depending on the condition of the subject being treated.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other embodiments of the present disclosure, the methods disclosed herein deliver the compound via various miscellaneous routes, for example, topical (i.e., transdermal) administration, mucosal administration (intranasal, vaginal), and/or inhalation.

Pharmaceutical compositions for topical administration may include the active compound formulated for a medicated application such as an ointment, paste, cream, or powder. Ointments include all oleaginous, adsorption, emulsion, and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream and petrolatum as well as any other suitable absorption, emulsion, or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch.” For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described. Likewise, the delivery of compounds using intranasal microparticle resins and lysophosphatidyl-glycerol compounds are also well-known in the pharmaceutical arts. Transmucosal drug delivery in the form of a polytetrafluoroetheylene support matrix may be adopted for use in accordance with the present disclosure.

The term aerosol refers to a colloidal system of finely divided solid of liquid particles dispersed in a liquefied or pressurized gas propellant. An aerosol for inhalation may consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In addition, in various embodiments, the compositions according to the disclosure may be formulated for delivery via any route of administration. The route of administration may refer to any administration pathway known in the art, including but not limited to aerosol, nasal, oral, transmucosal, transdermal, subcutaneous, or parenteral. Parenteral refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or in the form of lyophilized powders. In one embodiment, the administering is carried out intraperitoneally, orally, parenterally, nasally, subcutaneously, intravenously, intramuscularly, intracerebroventricularly, intraparenchymally, by inhalation, intranasal instillation, by implantation, by intracavitary or intravesical instillation, intraocularly, intraarterially, intralesionally, transdermally, topically, intradermally, intraplurally, intrathecally, or by application to mucous membranes.

Formulations include those suitable for oral, parenteral (including subcutaneous, intradermal, intramuscular, intravenous, and intraarticular), rectal, and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. In general, formulations may be prepared by uniformly and intimately bringing into association an active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of an active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary, or paste.

The present disclosure relates to, inter alia, compounds, methods, and compositions for inhibiting Prp8 intein expression or activity and methods of treating and/or preventing fungal infections, including, for example, treating subjects infected with one or more fungal infection, compounds for use in such methods, and methods for screening compounds that inhibit Prp8 intein splicing and kits for predicting the likelihood of Prp8 inhibition.

A second aspect of the present disclosure relates to a method of treating and/or preventing a fungal infection. The method comprises administering a Prp8 intein splicing inhibitor of formula (I)

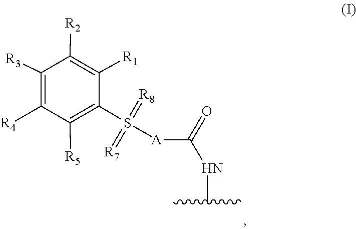

wherein R₁, R₂, R₃, R₄, and R₅ are independently selected from the group consisting of amine, amide, nitrogen oxide, C₁-C₂₃ alkyl, aryl, heteroaryl, carbocycle, heterocycle, oxygen, halogen, trifluoromethyl, and hydrogen, wherein the amine, amide, nitrogen oxide, C₁-C₂₃ alkyl, aryl, heteroaryl, carbocycle, heterocycle, oxygen, halogen, and trifluoromethyl can be optionally substituted with one or more halogen, hydrogen, C₁-C₃ alkyl, trifluoromethyl, or nitrogen oxide;

wherein R₆ and R₇ are independently selected from oxygen and hydrogen;

wherein A is independently selected from:

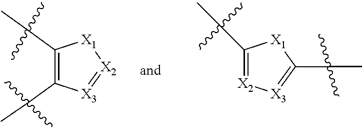

wherein

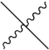

in A represent a point of attachment and wherein X₁, X₂, and X₃ are independently selected from carbon, nitrogen, sulfur, and oxygen; and

wherein [symbol] in formula (I) represents a point of attachment to at least one of:

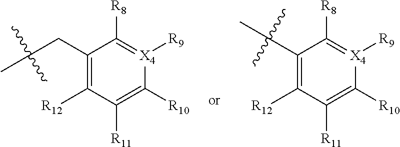

wherein X₄ is carbon or nitrogen;
wherein R₈, R₉, R₁₀, R₁₁, and R₁₂ are independently selected from hydrogen, halogen, trifluoromethyl, alkyl, and nitrogen oxide, under conditions effective to treat and/or prevent a fungal infection.

This aspect is carried out in accordance with the previously described aspect.

In one embodiment, one or more of R₁, R₂, R₃, R₄, and R₅ is hydrogen, halogen, or trifluoromethyl. In another embodiment, one or more of R₁, R₂, R₃, R₄, and R₅ is chlorine. In one embodiment, one or more of X₁, X₂, and X₃ is N. In one embodiment, one or more of X₁, X₂, and X₃ is S. In one embodiment, one or more of R₈, R₉, R₁₀, R₁₁, and R₁₂ is hydrogen, fluorine, chlorine, bromine, trifluoromethyl, methyl, or nitrogen oxide.

In one embodiment, in the Prp8 intein splicing inhibitor of formula (I), the Prp8 intein splicing inhibitor has the formulae (Ia) through (Id″):

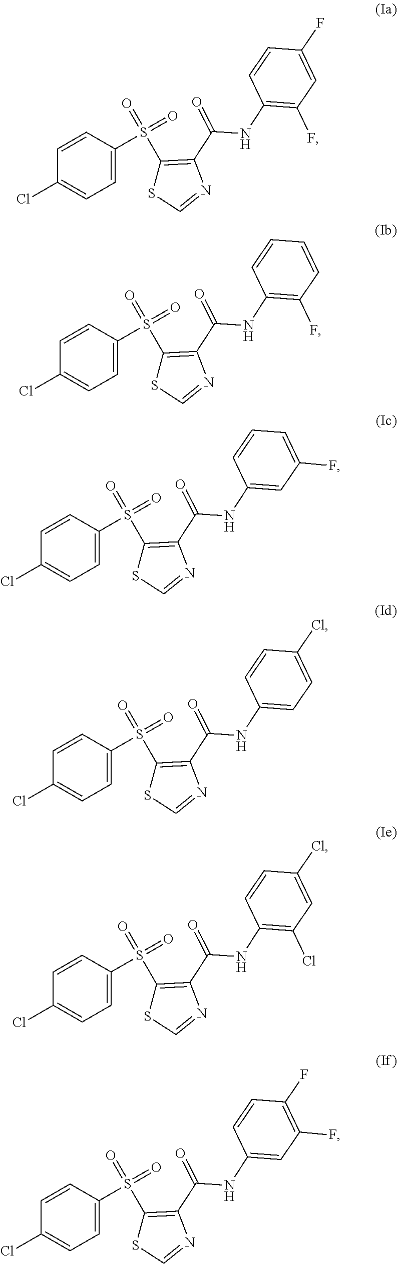 [SMILES: O=C(Nc1ccc(F)cc1F)c1ncsc1S(=O)(=O)c1ccc(Cl)cc1 ; O=C(Nc1ccccc1F)c1ncsc1S(=O)(=O)c1ccc(Cl)cc1 ; O=C(Nc1cccc(F)c1)c1ncsc1S(=O)(=O)c1ccc(Cl)cc1 ; O=C(Nc1ccc(Cl)cc1)c1ncsc1S(=O)(=O)c1ccc(Cl)cc1 ; O=C(Nc1ccc(Cl)cc1Cl)c1ncsc1S(=O)(=O)c1ccc(Cl)cc1 ; O=C(Nc1ccc(F)c(F)c1)c1ncsc1S(=O)(=O)c1ccc(Cl)cc1]

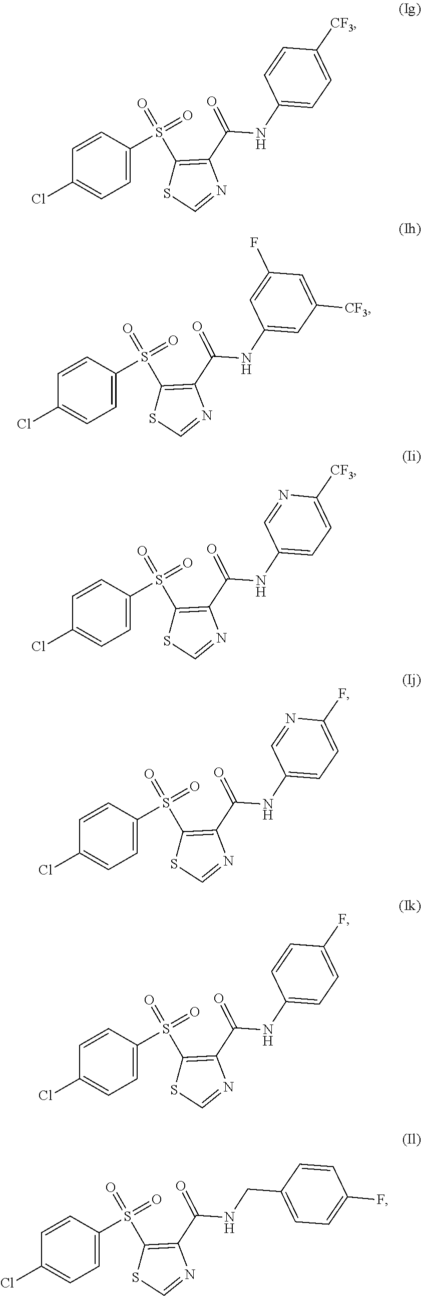 [SMILES: O=C(Nc1ccc(C(F)(F)F)cc1)c1ncsc1S(=O)(=O)c1ccc(Cl)cc1 ; O=C(Nc1cc(F)cc(C(F)(F)F)c1)c1ncsc1S(=O)(=O)c1ccc(Cl)cc1 ; O=C(Nc1ccc(C(F)(F)F)nc1)c1ncsc1S(=O)(=O)c1ccc(Cl)cc1 ; O=C(Nc1ccc(F)nc1)c1ncsc1S(=O)(=O)c1ccc(Cl)cc1 ; O=C(Nc1ccc(F)cc1)c1ncsc1S(=O)(=O)c1ccc(Cl)cc1 ; O=C(NCc1ccc(F)cc1)c1ncsc1S(=O)(=O)c1ccc(Cl)cc1]

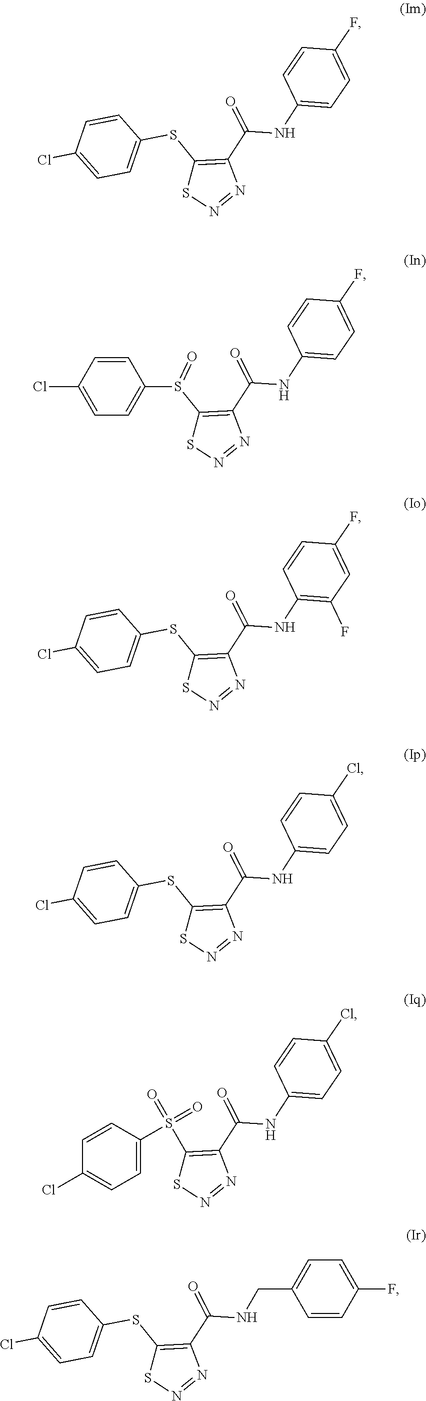 [SMILES: O=C(Nc1ccc(F)cc1)c1nnsc1Sc1ccc(Cl)cc1 ; O=C(Nc1ccc(F)cc1)c1nnsc1S(=O)c1ccc(Cl)cc1 ; O=C(Nc1ccc(F)cc1F)c1nnsc1Sc1ccc(Cl)cc1 ; O=C(Nc1ccc(Cl)cc1)c1nnsc1Sc1ccc(Cl)cc1 ; O=C(Nc1ccc(Cl)cc1)c1nnsc1S(=O)(=O)c1ccc(Cl)cc1 ; O=C(NCc1ccc(F)cc1)c1nnsc1Sc1ccc(Cl)cc1]

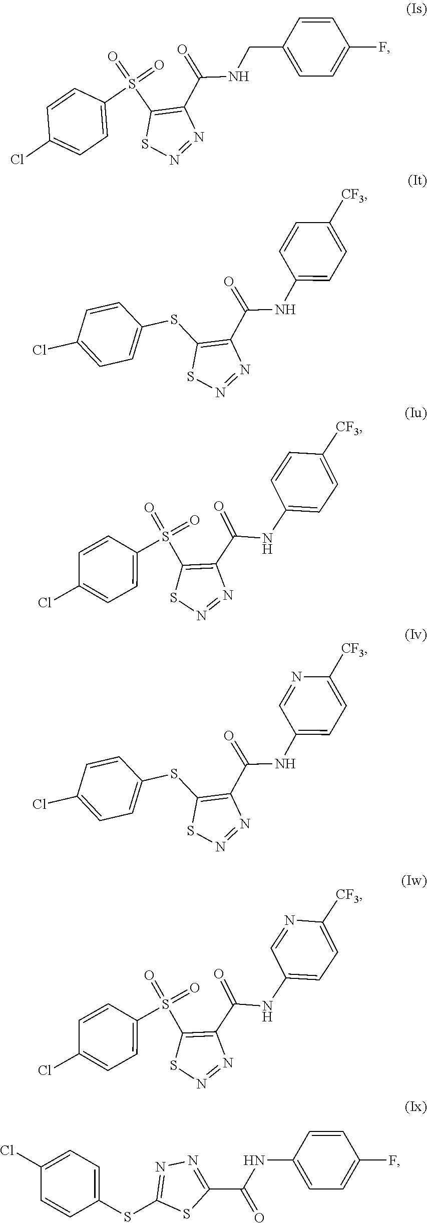 [SMILES: O=C(NCc1ccc(F)cc1)c1nnsc1S(=O)(=O)c1ccc(Cl)cc1 ; O=C(Nc1ccc(C(F)(F)F)cc1)c1nnsc1Sc1ccc(Cl)cc1 ; O=C(Nc1ccc(C(F)(F)F)cc1)c1nnsc1S(=O)(=O)c1ccc(Cl)cc1 ; O=C(Nc1ccc(C(F)(F)F)nc1)c1nnsc1Sc1ccc(Cl)cc1 ; O=C(Nc1ccc(C(F)(F)F)nc1)c1nnsc1S(=O)(=O)c1ccc(Cl)cc1 ; O=C(Nc1ccc(F)cc1)c1nnc(Sc2ccc(Cl)cc2)s1]

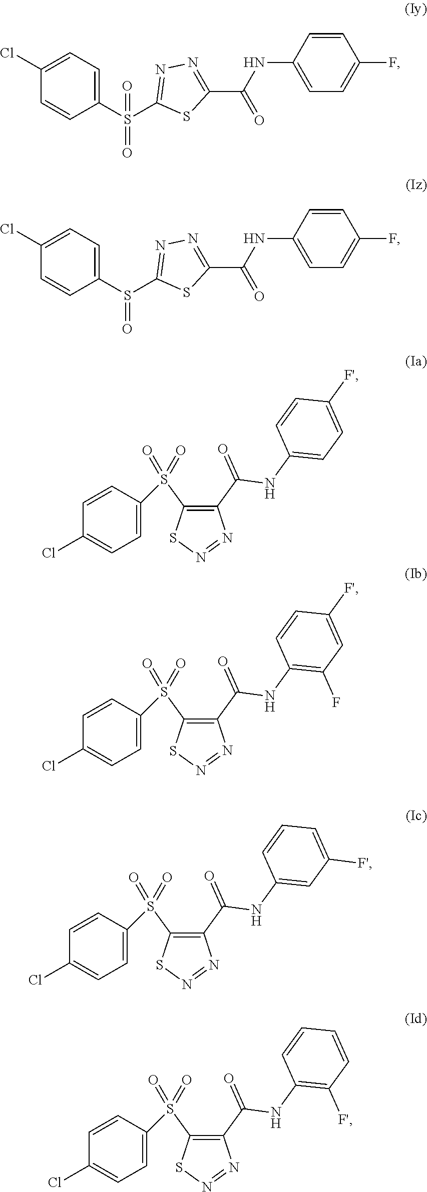

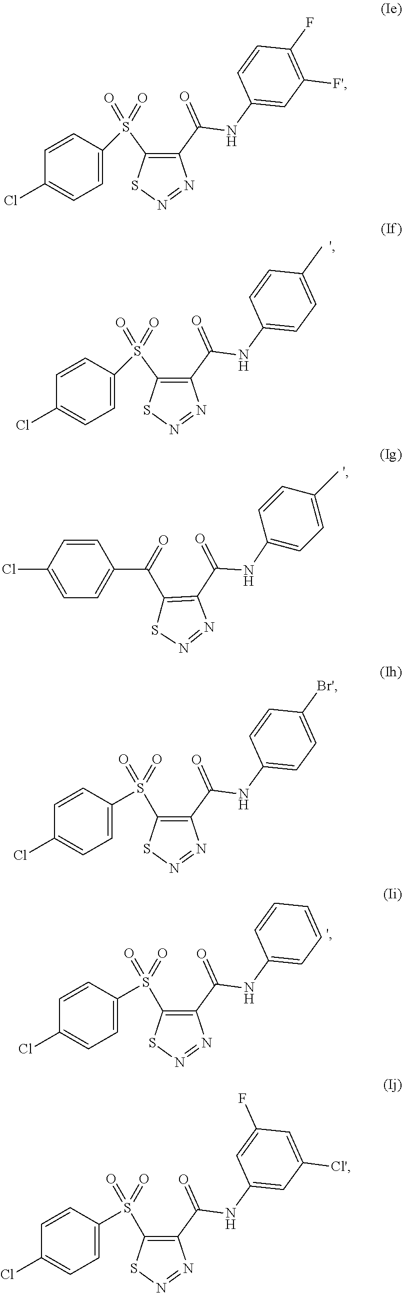

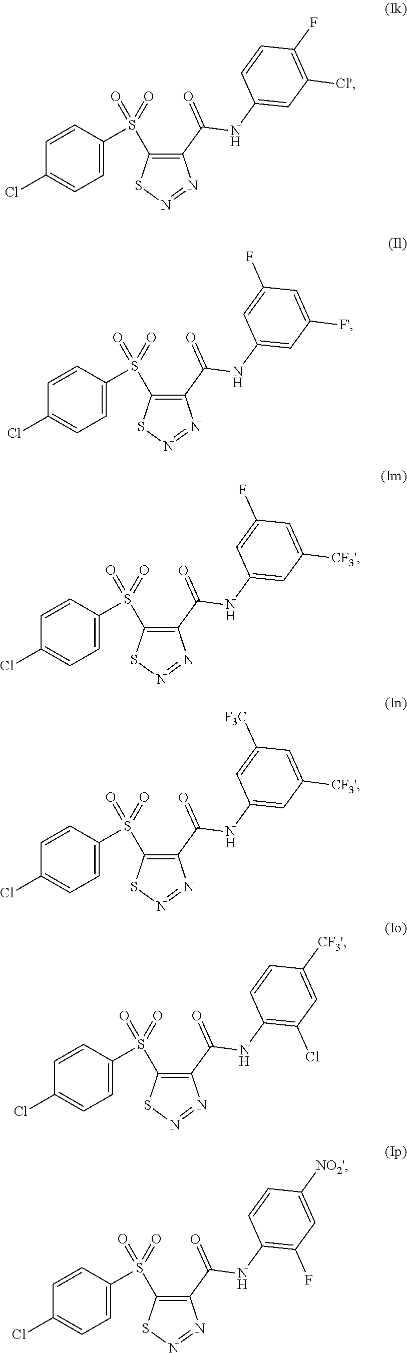

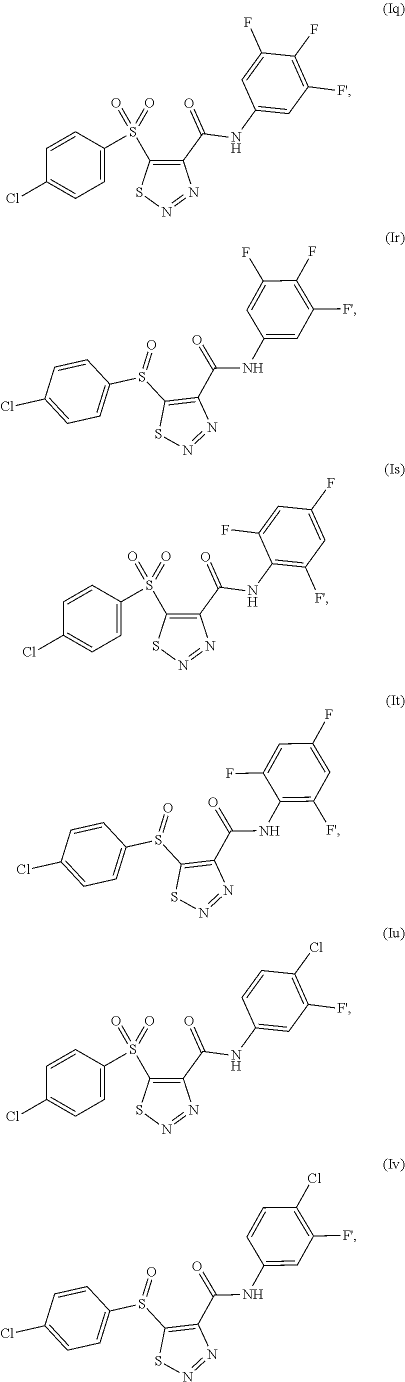

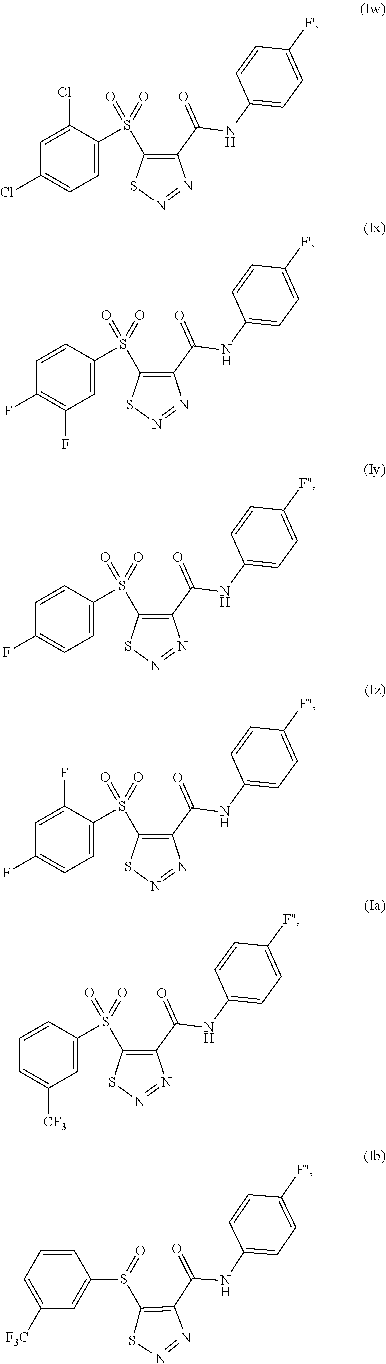

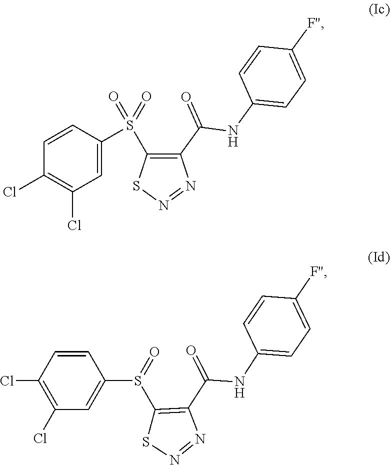

or a pharmaceutically acceptable salt thereof.

In one embodiment, the contacting one or more cells that have been infected with a compound of formula (I) comprises administering the compound to a subject. In another embodiment, the contacting one or more cells that have been infected with a compound of formula (I) comprises administering the compound to a subject and the subject is a mammal. In yet another embodiment, the contacting one or more cells that have been infected with a compound of formula (I) comprises administering the compound to a subject and the subject is a human. In one embodiment, the cells comprise tissue that has been removed from a mammal. In another embodiment, the cells comprise tissue that has been removed from a human.

The fungal infection may be derived from any pathogenic fungi. In one embodiment, the fungal infection is derived from a pathogenic fungi selected from, but not limited to, Cryptococcus, Aspergillus, Blastomyces, Coccidiodes, Histoplasma, Phycomyces, Tinea corporis, Tinea unguis, Sporothrix schenckii, Pneumocystis carinii, Candida, or any combination thereof. In another embodiment, the fungal infection is derived from a pathogenic fungi selected from, for example, Cryptococcus neoformans, Cryptococcus gatthi, Aspergillus fumigatus or any combination thereof. For example, the compound may be formula (I) or a pharmaceutically acceptable salt thereof and the pathogenic fungi may be Cryptococcus, the compound may be formula (I) or a pharmaceutically acceptable salt thereof and the pathogenic fungi may be Aspergillus, the compound may be formula (I) or a pharmaceutically acceptable salt thereof and the pathogenic fungi may be Blastomyces, the compound may be formula (I) or a pharmaceutically acceptable salt thereof and the pathogenic fungi may be Coccidiodes, the compound may be formula (I) or a pharmaceutically acceptable salt thereof and the pathogenic fungi may be Histoplasma, the compound may be formula (I) or a pharmaceutically acceptable salt thereof and the pathogenic fungi may be Phycomyces, the compound may be formula (I) or a pharmaceutically acceptable salt thereof and the pathogenic fungi may be Tinea corporis, the compound may be formula (I) or a pharmaceutically acceptable salt thereof and the pathogenic fungi may be Tinea unguis, the compound may be formula (I) or a pharmaceutically acceptable salt thereof and the pathogenic fungi may be Sporothrix schenckii, the compound may be formula (I) or a pharmaceutically acceptable salt thereof and the pathogenic fungi may be Pneumocystis carinii, the compound may be formula (I) or a pharmaceutically acceptable salt thereof and the pathogenic fungi may be Candida, or any combination thereof. Alternatively, the compound may be formula (I) or a pharmaceutically acceptable salt thereof and the pathogenic fungi may be Cryptococcus neoformans, the compound may be formula (I) or a pharmaceutically acceptable salt thereof and the pathogenic fungi may be Cryptococcus gattii, the compound may be formula (I) or a pharmaceutically acceptable salt thereof and the pathogenic fungi may be Aspergillus fumigatus, or any combination thereof.

In one embodiment, the method further includes selecting a subject that has a fungal infection or is at risk of developing a fungal infection. In one embodiment, the fungal infection is Aspergillosis, Blastomycosis, Candidiasis, Candida auris, Coccidioidomycosis, C. neorformans infection, C. gattii infection, fungal eye infections, fungal nail infections, Histoplasmosis, Mucormycosis, Mycetoma, Pneumocystis pneumonia, ringworm, sporotrichosis, Paracoccidioidomycosis, Talaromycosis (formerly Penicilliosis), or any other fungal infection, or any combination thereof.

For purposes of this and other aspects of the disclosure, the target “subject” encompasses any vertebrate, such as an animal, preferably a mammal, more preferably a human. In the context of administering a composition of the disclosure for purposes of preventing and/or treating a fungal infection in a subject comprising in a subject, the target subject encompasses any subject that has a fungal infection or is at risk of developing a fungal infection. Particularly susceptible subjects include those exposed to a fungal, and may include infants, juveniles, adults, or elderly adults that have a fungal infection or are at risk of having a fungal infection. In one embodiment, the subject is an infant, a juvenile, or an adult. In one embodiment, the method is performed in a subject having a preexisting condition or, alternatively, may be performed in a subject having no preexisting condition. The method may also be performed on a subject who has been previously treated for a fungal infection.

In any embodiment described herein, any of the compounds identified may be administered before confirmation of the presence of infection in any cell or cells or sample from a subject, including a human subject or other mammalian subject, with the effect of preventing infection. For example, a sample or subject may be suspected of having been exposed to a pathogenic fungi and administration of one or more of the foregoing compounds may be applied so as to prevent replication of the fungi should contact otherwise sufficient to cause fungal replication or infection of the cells have been present. Notwithstanding an apparent mechanism of action of the foregoing compounds in preventing fungal replication, preventing or reversing infection, spread, or illness, as disclosed herein, the present disclosure is not limited to preventing fungal replication, preventing or reversing infection, spread, or illness according to any particular mechanism of action, including any apparent mechanism of action disclosed herein. Any or all of the foregoing compounds may prevent fungal replication, prevent or reverse infection, spread, or illness, by any mechanism of action and still be included within the present disclosure, even if not by a mechanism of action disclosed herein. Anti-fungal effects of formula (I) (e.g., any compound of formulae (Ia) through (Id″)) may occur by any mechanism, and identification of potential mechanism(s) of action identified herein in no way excludes mechanisms of action by which these compounds may exert anti-fungal effects, other than or including any mechanism disclosed herein, from falling within the scope of subject matter disclosed herein, without limitation as to any mechanism for such anti-fungal effects not explicitly identified mechanistically.

The actual dosage amount of a composition of the present disclosure administered to a subject (e.g., an animal or human patient) in the methods described herein can be determined by physical and physiological factors such as body weight, severity of condition, the type of disease being treated, previous or concurrent therapeutic interventions, idiopathy of the patient, and route of administration. Depending upon the dosage and the route of administration, the number of administrations of a preferred dosage and/or an effective amount may vary according to the response of the subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one of ordinary skill in the art of preparing such pharmaceutical formulations, and as such, a variety of dosages and treatment regimens may be desirable.

For example, a dose may comprise from between about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, can be administered, based on the numbers described above.

In one embodiment, the administering is provided in an amount between about 0.01 μg/ml and about 5.0 μg/ml. For example, the amount administered may be about 0.01 μg/ml, about 0.02 μg/ml, about 0.03 μg/ml, about 0.04 μg/ml, about 0.05 μg/ml, about 0.06 μg/ml, about 0.07 μg/ml, about 0.08 μg/ml, about 0.09 μg/ml, about 0.10 μg/ml, about 0.15 μg/ml, about 0.20 μg/ml, about 0.25 μg/ml, about 0.30 μg/ml, about 0.35 μg/ml, about 0.40 μg/ml, about 0.45 μg/ml, about 0.50 μg/ml, about 0.75 μg/ml, about 1.00 μg/ml, about 1.25 μg/ml, about 1.50 μg/ml, about 1.75 μg/ml, about 2.00 μg/ml, about 2.25 μg/ml, about 2.50 μg/ml, about 2.75 μg/ml, about 3.00 μg/ml, about 3.25 μg/ml, about 3.50 μg/ml, about 3.75 μg/ml, about 4.00 μg/ml, about 4.25 μg/ml, about 4.50 μg/ml, about 4.75 μg/ml, about 5.00 μg/ml, or any amount therebetween.

The compound of formula (I), may, in one embodiment, be administered under an extended dosing and/or an intermittent dosing protocol.

An extended dosing protocol as described herein may include administering the compound of formula (I) more than once, and for a period of time beyond a single dose or a short dosing protocol. For example, an extended dosing protocol may include administering one or more doses of the compound of formula (I) to a sample or a subject each consecutive day for a period of more than one day. Alternatively, an extended dosing protocol may, for example, include administering one or more doses of the compound of formula (I) to a sample or a subject at a predetermined interval with one or more days between each administration.

An intermittent dosing protocol as described herein may include administering the compound of formula (I) more than once, and optionally at periodic intervals that are determined to be useful in inducing the effect of treating and/or preventing a fungal infection. An intermittent dosing protocol as described herein may be useful in managing and mitigating toxicity risks to a subject or a sample, as it may administer more than one dose but also provides a period of time between each dose is administered sufficient to reduce toxicity in a subject or a sample. Under an intermittent dosing protocol, the compound of formula (I) may be administered in one or more doses for a set period of time (“on period”) followed by a set period of time with no doses being administered (“off period”), followed by repeating the “on period” and “off period” one or more times for a sustained period of time effective to treat and/or prevent a fungal infection and/or inhibit Prp8 intein expression or activity.

In one embodiment, the method includes repeating administration of the compound of formula (I) any number of times necessary to create a desired effect. For example, the compound of formula (I) may be administered once daily for a set period of time (i.e., an extended dosing protocol) or may be administered for one or more “on periods” where each “on period” may be followed by an “off period” (i.e., an intermittent dosing protocol). In one embodiment, the compound of formula (I) is administered once daily for a period of time of at least about 1 week. For example, the compound of formula (I) may be administered once daily for about 1 week, about 2 weeks, about 3 weeks, about 4 weeks, about 5 weeks, about 6 weeks, about 7 weeks, about 8 weeks, about 9 weeks, about 10 weeks, about 12 weeks, about 15 weeks, about 20 weeks, about 25 weeks, about 30 weeks, about 35 weeks, about 40 weeks, about 45 weeks, about 50 weeks, about 52 weeks, about 1 year, or more than about 1 year. In one embodiment, the compound of formula (I) is administered for a time of between about 1 week and about 12 weeks.

In one embodiment, the compound of formula (I) is administered under an intermittent dosing protocol, for example, once daily for one week (i.e., “on period”), followed by 3 weeks with no administration (i.e., “off period”), and this may optionally be repeated. Alternatively, in one embodiment, the compound of formula (I) is administered under an intermittent dosing protocol, for example, once daily for one week (i.e., “on period”), followed by 8 weeks with no administration (i.e., “off period”), and this may optionally be repeated. In another embodiment, the compound of formula (I) is administered under an intermittent dosing protocol, for example, once daily for one week (i.e., “on period”), followed by 6 months off with no administration (i.e., “off period”), and this may optionally be repeated.

The terms dose and dosage are used interchangeably herein. A dose refers to the amount of active ingredient given to an individual at each administration. The dose will vary depending on a number of factors, including the range of normal doses for a given therapy, frequency of administration, size and tolerance of the individual, severity of the condition, risk of side effects, and the route of administration. One of skill will recognize that the dose can be modified depending on the above factors or based on therapeutic progress. The term dosage form refers to the particular format of the pharmaceutical or pharmaceutical composition, and depends on the route of administration. Dosage, toxicity, and therapeutic efficacy of the agents or compositions of the present disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices may be desirable. While compositions that exhibit toxic side effects may be used, care should be taken to design a delivery system that targets such compositions to the site of affected tissue in order to minimize potential damage to uninfected cells and, thereby, reduce side effects.

In one embodiment, the treatment comprises administering said formula (I) (for example, administering a compound of formulae (Ia)-(Id″)) to a subject transdermally, intradermally, parenterally, subcutaneously, intravenous injection, intra-arterial injection, intramuscular injection, intrapleurally, intraperitoneally, intrathecally, or by application to a mucous membrane, orally, by inhalation, by intranasal instillation, or topically.

In addition, compounds according to the present disclosure may be administered alone or in combination with other agents, including other compounds of the present disclosure. Certain compounds according to the present disclosure may be effective for enhancing the biological activity of certain agents according to the present disclosure by reducing the metabolism, catabolism or inactivation of other compounds and as such, may be co-administered for this intended effect.

In one embodiment, the method further comprises administering one or more additional agents and/or treatments which prevent or treat fungal infections and/or a condition resulting from fungal infections. In one embodiment the one or more additional agent is a compound selected from the group consisting of 5-fluorocytosine (5-FC), itraconazole, fluconazole, amphotericin B, anidulafungin, micafungin, caspofungin, posaconazole, and voriconazole. In another embodiment, the method further comprises repeating the administering of the Prp8 intein splicing inhibitor of formula (I) (e.g., formulae (Ia)(Id″)).

As used herein, the phrase “therapeutically effective amount” means an amount of active compound or pharmaceutical agent (e.g., a Prp8 intein splicing inhibitor) that elicits the biological or medicinal response that is being sought in a tissue, system, animal, individual, or human by a researcher, veterinarian, medical doctor, or other clinician. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment. The term “treatment” or “treat” may include effective inhibition, suppression, or cessation of fungal infection symptoms so as to prevent or delay the onset, retard the progression, or ameliorate the symptoms of the infection caused by a fungus. In one embodiment, the disclosure includes administering a composition comprising an effective amount of a Prp8 intein splicing inhibitor, such as a that of formula (I), to an individual in need of prophylaxis and/or therapy of a condition that would benefit from inhibition of Prp8 intein splicing. In one embodiment, the administering of the Prp8 intein splicing inhibitor is such that one or more symptoms of a condition are improved.

One goal of treatment is the amelioration, either partial or complete, either temporary or permanent, of patient symptoms. Any amelioration is considered successful treatment. This is especially true as amelioration of some magnitude may allow reduction of other medical or surgical treatment which may be more toxic or invasive to the patient.

Administration of a compound described herein may result in a reduction in one or more symptoms by at least 10%, 20%, 30%, 50% or greater, up to a 75-90/6), or 95% or greater, reduction in the one or more symptoms in a subject, compared to placebo-treated or other suitable control subjects, or any other suitable reference. In one embodiment, the method includes administering a composition comprising a therapeutically effective amount of a compound described herein.

As used herein a sample may include any sample obtained from a living system or subject, including, for example, blood, serum, and/or tissue. In one embodiment, a sample is obtained through sampling by minimally invasive or non-invasive approaches (for example, by urine collection, stool collection, blood drawing, needle aspiration, and other procedures involving minimal risk, discomfort, or effort). Alternatively, samples may be gaseous (for example, breath that has been exhaled) or liquid fluid. Liquid samples may include, for example, urine, blood, serum, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, nasal excretions, and other liquids. Samples may also include a clinical sample such as serum, plasma, other biological fluid, or tissue samples, and also includes cells in culture, cell supernatants, and cell lysates. In one embodiment, the sample is selected from the group consisting of whole blood, serum, urine, and nasal excretion. Samples may be in vivo or ex vivo.

Administration of a Prp8 intein splicing inhibitor, such as that of formula (I) (e.g., and compound of formulae (Ia) through (Id″)), can be performed in conjunction with conventional therapies that are intended to treat and/or prevent a fungal infection. For example, a composition comprising the compound of formula (I) could be administered prior to, concurrently, or subsequent to conventional therapies known to those skilled in the art for prophylaxis or therapy for conditions that may benefit from inhibition of Prp8 intein splicing. Such therapies include but are not limited to combining treatment with other pharmaceutical agent(s) known to be effective against the particular condition being treated, behavioral and physical therapies, cognitive therapies, and the like. In one embodiment, an additional agent may be administered in addition to the compound of formula (I).

As used herein, the term “simultaneous” therapeutic use refers to the administration of at least one additional agent beyond the compound of formula (I) (e.g., and compound of formulae (Ia) through (Id″)), for example, agents administered before, during, or after the compound of formula (I), optionally, by the same route and at the same time or at substantially the same time. As used herein, the term “separate” therapeutic use refers to an administration of at least one additional agent beyond the compound of formula (I), for example, agents administered before, during, or after administration of compound of formula (I), at the same time or at substantially the same time by different routes.

As used herein, the term “sequential” therapeutic use refers to administration of at least one additional agent beyond the compound of formula (I) (e.g., and compound of formulae (Ia) through (Id″)), for example, agents administered before, during, or after administration of the compound of formula (I), at different times, the administration route being identical or different. More particularly, sequential use refers to the whole administration of the additional agent before administration of compound of formula (I). It is thus possible to administer the additional agent over several minutes, hours, or days before applying the compound of formula (I). In one embodiment, the additional agent is administered before, during, or after the compound of formula (I).

In one embodiment, the additional agent may include, for example, any treatments that are useful in treating a subject having or at risk of having a fungal infection. For example, any treatments known by those skilled in the art for preventing and/or treating fungal infections, could be useful as an additional agent. The additional agent may, for example, be one or more antibiotic compound; one or more antimicrobial compound; one or more antibody; one or more biocidal agent; one or more nanoparticle; one or more self-assembling nanoparticle; one or more viral particle; one or more bacteriophage particle; one or more bacteriophage DNA; genetic material including but not limited to a plasmid, RNA, mRNA, siRNA, and an aptamer; one or more chemotherapy agent; one or more growth factor; one or more synthetic scaffold including but not limited to hydrogel and others; one or more natural scaffold including but not limited to collagen gel and decellularized tissue (whole, dissolved, denatured, or powdered); one or more electrode; one or more drug or pharmaceutical compound including but not limited to an anti-inflammatory agent, an inflammatory agent, a pain blocking agent, and a numbing agent; one or more microbes; and one or more bacteria.

Any of the compounds disclosed herein may be used in accordance with the present disclosure to prevent infection of a subject, such as a human subject, or other mammalian subject, with a fungal infection such as one of the types of fungal infection identified in the above paragraphs. For example, any amount of therapeutic of the present disclosure, or a pharmaceutically acceptable salt of the compound, optionally in combination with a pharmaceutically acceptable excipient, carrier, or additive, sufficient to prevent fungal infection, may be administered to such subject. Use of any one or more of the foregoing compounds to prevent infection with any one or more fungal infections, including those specifically identified above, is explicitly contemplated and hereby included in the present disclosure. In one embodiment, the fungal infection is prevented. In another embodiment, the fungal infection is treated.

A third aspect of the present disclosure relates to a method of inhibiting Prp8 intein expression or activity in a cell or tissue. The method comprises administering a compound of formula (I):

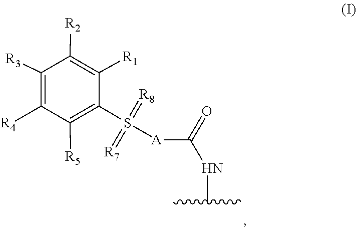

wherein R₁, R₂, R₃, R₄, and R₅ are independently selected from the group consisting of amine, amide, nitrogen oxide, C₁-C₂₃ alkyl, aryl, heteroaryl, carbocycle, heterocycle, oxygen, halogen, trifluoromethyl, and hydrogen, wherein the amine, amide, nitrogen oxide, C₁-C₂₃ alkyl, aryl, heteroaryl, carbocycle, heterocycle, oxygen, halogen, and trifluoromethyl can be optionally substituted with one or more halogen, hydrogen, C₁-C₃ alkyl, trifluoromethyl, or nitrogen oxide;

wherein R₆ and R₇ are independently selected from oxygen and hydrogen;

wherein A is independently selected from:

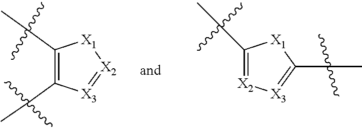

wherein

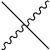

in A represent a point of attachment and wherein X₁, X₂, and X₃ are independently selected from carbon, nitrogen, sulfur, and oxygen; and

wherein [symbol] in formula (I) represents a point of attachment to at least one of:

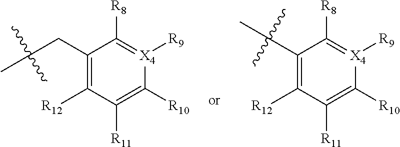

wherein X₄ is carbon or nitrogen;
wherein R₈, R₉, R₁₀, R₁₁, and R₁₂ are independently selected from hydrogen, halogen, trifluoromethyl, alkyl, and nitrogen oxide, under conditions effective to inhibit Prp8 intein expression or activity in a cell or tissue.

This aspect is carried out in accordance with the previously described aspects.

In one embodiment, the inhibiting Prp8 intein expression or activity is an inhibition of Prp intein splicing.

In another embodiment, the method further includes providing a cell or tissue containing Prp8 intein expression or activity. In one embodiment, the cell or tissue is from a subject having a fungal infection or from a subject at risk of having a fungal infection in accordance with the fungal infections as described herein.

In one embodiment, the method further includes detecting a Prp8 intein splicing level in the subject.

In another embodiment, the method further includes comparing the detected Prp8 intein splicing level in the subject to a Prp8 intein splicing level standard for a subject not having and/or not at risk of having a fungal infection.

A fourth aspect of the present disclosure relates to a method for screening for compounds that inhibit Prp8 intein splicing comprising an assay. The assay includes providing a GFP-Prp8 fusion protein; treating the GFP-Prp8 fusion protein with an intein splicing buffer; reacting the treated GFP-Prp8 fusion protein with one or more reagent; and detecting Prp8 intein splicing activity in a compound.

This aspect is carried out in accordance with the previously described aspects.

In one embodiment, the GFP-Prp8 fusion protein comprises a Prp8 intein, a first GFP residue, and a second GFP residue.

In one embodiment, the one or more reagent is a reducing reagent. The reducing reagent may be selected from, for example, Tris (2-carboxyethyl) phosphine, lithium aluminum hydride, nascent (atomic) hydrogen, hydrogen without or with a suitable catalyst, sodium amalgam, sodium-lead alloy, zinc amalgam, diborane, sodium borohydride, compounds containing the Fe²⁺ ion and/or an Sn²⁺ ion, sulfur dioxide, sulfite compounds, dithionates, thiosulfates, iodides, hydrogen peroxide, hydrazine, diisobutylaluminum hydride, oxalic acid, formic acid, ascorbic acid, reducing sugars, phosphites, hypophosphites, phosphorous acid, dithiothreitol, carbon monoxide, and cyanides.

In one embodiment, the compound treated with the one or more reagent triggers Prp8 intein splicing.

In one embodiment, the compound treated with the one or more reagent inhibits Prp8 intein splicing.

A fifth aspect of the present disclosure relates to a kit for predicting the likelihood of Prp8 inhibition. The kit comprises one or more agents that specifically recognize Prp8 intein expression or activity and a label that detects said recognition of Prp8 intein expression or activity by said one or more agents.

This aspect is carried out in accordance with the previously described aspects.

In one embodiment, the agent detects Prp8 intein levels.

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense.

The present disclosure may be further illustrated by reference to the following examples.

### EXAMPLES

The following examples are intended to illustrate, but by no means are intended to limit, the scope of the present disclosure as set forth in the appended claims.

### Example 1—Materials and Methods

Compounds—Tris(2-carboxyethyl)phosphine (TCEP) and cisplatin were from Sigma-Aldrich. Compounds 6G-318S, 6G-319S, 12G-305S, 6G-313S, and 12G-315S were from Key Organics.

Cloning, expression and purfication—The split GFP-Prp8i was constructed using a mega-PCR mutagenesis approach. Brecher et al., “A Conformational Switch High-Throughput Screening Assay and Allosteric Inhibition of the Flavivirus NS2B-NS3 Protease,” PLoS Pathogens 13(5):e1006411 (2017), which is hereby incorporated by reference in its entirety. Briefly, a PCR DNA fragment representing the Prp8 intein (italic) flanked by GFP overlapping sequences (underlined) was amplified using the C. gattii genomic DNA as a template, with primers prp8_N_GFP_F (CCTTGTTAATCGTATCGTGTTAAAAGGTTGTCTGCAGAACGGTACCCG) (SEQ ID NO: 5) and prp8_C_GFP_R (CGAGAATGTTTCCATCTTCTTTAAAATCTGAGTTGTGTAATACCAAATAGTCATGAC) (SEQ ID NO: 6). The overlapping PCR product was then used as a megaprimer for PCR mutagenesis with the split GFP-RecA intein plasmid (Chan et al. “Exploring Intein Inhibition by Platinum Compounds as an Antimicrobial Strategy,” The Journal of Biological Chemistry 291(43):22661-22670 (2016), which is hereby incorporated by reference in its entirety) as a template. After digestion with Dpn I restriction enzyme, the PCR product was transformed into DH5α Escherichia coli. Clones were verified by plasmid DNA sequencing. The expression, refolding and purification of the GFP-Prp8i fusion protein were carried out as described previously. Chan et al. “Exploring Intein Inhibition by Platinum Compounds as an Antimicrobial Strategy,” The Journal of Biological Chemistry 291(43):22661-22670 (2016), which is hereby incorporated by reference in its entirety. One exception is that prior to refolding, the denatured and solubilized GFP-Prp8i inclusion body was treated with 5,5′-dithiobis-(2-nitrobenzoic) acid (DTNB) to further prevent GFP-Prp8i self-splicing, by reacting with free cysteine at the intein active site.

The Cga MIG Prp8 and the Prp8 intein using pXI and pET28a vectors were constructed, expressed, and purified as described previously. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. The Flag-Prp8 intein and mutant constructs were cloned or synthesized into the pXL1-PTEF1 vector, as described previously (Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety) and transformed into the C. neoformans H99 strain, using a Bio-Rad electroporator.

Split GFP-Prp8i intein splicing assay—Using black and clear bottom 96-well plates, the DPNB-treated, refolded and concentrated GFP-Prp8i fusion protein (0.5 μM) was incubated with compounds or dimethylformamide (DMF) solvent control for 30 min at 25° C. in a 100 μl intein splicing buffer (20 mM Bis-tris propane, 0.5 M NaCl, 0.5 M arginine, 1 mM EDTA, and 0.05% CHAPS). Then 100 μl 2×TCEP was added to a final concentration of 20 μM. The reactions were incubated at 25° C. overnight. GFP-Prp8i splicing was monitored using a Bio-Tek FL800 microplate reader with excitation and emission wavelengths of 485 nm and 528 nm, respectively. Two readings were taken, one prior to addition of TCEP (I₀) and one after incubation overnight with TCEP (I_{f}). Fluorescence/luminescence differences (ΔFluorescence or ΔLuminescence) were defined as I_{f}−I₀ for each well. Relative GFP fluorescence-Prp8 (FLU) or RLuc-Prp8 luminescence (RLU, see below) was calculated by the following formula: % FLU (or % RLU)=(I_{f-comp}−I_{0-comp})/(I_{f-DMF}−I_{0-DMF})×100. DMF control served as 100% splicing activity.

Split luciferase Prp8 intein splicing assay—The split RLuc-Prp8 fusion protein was carried out and monitored using a Veritas luminometer, as described previously. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety.

Trypsin and papain protease inhibition—An Abz FRET substrate (Abz-RRRRSAG-nTyr) developed previously (Li et al., “Existing Drugs as Broad-Spectrum and Potent Inhibitors for Zika Virus by Targeting NS2B-NS3 Interaction,” Cell Research 27(8):1046-1064 (2017), which is hereby incorporated by reference in its entirety) was used as the substrate for inhibition of trypsin and papain proteases. In black and clear-bottom 96-well plates, 1 μg trypsin (Sigma-Aldrich) was incubated with DMF or compounds at 400 μM at 25° C. for 30 min in a 70 μl reaction buffer containing 20 mM Tris, pH 8.0, 100 mM NaCl, 5% Glycerol, and 0.05% CHAPS. For papain, 1 μg Papain (Sigma-Aldrich) was incubated with compounds at 400 μM at 25° C. for 30 min in a 70 μl reaction buffer composed of 1.1 mM EDTA, 0.067 mM β-mercaptoethanol, 5.5 mM cysteine. Substrate cleavage was monitored using a Bio-Tek FL800 microplate reader with excitation and emission wavelengths of 360 nm and 420 nm, respectively.

The MIG-Prp8 and Prp8-GFP in-gel splicing assays—The MIG Prp8 and Prp8-GFP splicing assays were carried out as described. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. The samples were analyzed in SDS-PAGE without boiling. The gels were quantified using GFP fluorescence and Coomassie blue staining with the Bio-Rad Gel Doc EZ.

Cytotoxicity—Cytotoxicity assays were carried out using human lung carcinoma cell line A549, as described (Chen et al., “Selective Inhibition of the West Nile Virus Methyltransferase by Nucleoside Analogs,” Antiviral Research 97(3):232-239 (2013), which is hereby incorporated by reference in its entirety). Briefly, the A549 cells (5×10³) were incubated with/without compounds in a 96-well plate at 37° C. for 48 h. Cell viability was determined using the MTT assay (ATCC) as was described previously (Chen et al., “Selective Inhibition of the West Nile Virus Methyltransferase by Nucleoside Analogs,” Antiviral Research 97(3):232-239 (2013) and Brecher et al., “Identification and Characterization of Novel Broad-Spectrum Inhibitors of the Flavivirus Methyltransferase,” ACS Infectious Diseases 1(8):340-349 (2015), both of which are hereby incorporated by reference in their entirety). N=3.

Western blot (WB)—Fresh colonies of Cryptococcus neoformans were grown in yeast extract peptone dextrose (YPD) broth in an environmental shaker (250 rpm) overnight at 30° C. The fungal cells (5 ml) were diluted in 125 ml YPD broth and grown for 1.5 h to a final cell density of 0.2 at OD₅₃₀. The cells were distributed to test tubes (3 ml/tube). Then 3 ml of YPD medium containing DMF or 2-fold diluted compounds was added to the cells. The mixtures were incubated at 30° C. in a shaker at 250 RPM for up to 18 h. Samples were spun down and pellets were frozen for WB analysis.

Cryptococcal cells treated with DMF or compounds were washed twice with PBS buffer. Cells were spun down and supernatant was removed. For a 6-ml cell pellet, 30 μl of complete protease inhibitor cocktail in PBS was added to the cells, followed by addition of 400 μl lysis buffer (50 mM Tris, pH 8.0, 500 mM NaCl, 10% glycerol) and 500 μl glass beads. The fungal cells were lysed by rigorous beating for 2 min 3 times. The cell lysate was spun down. Supernatant was mixed with SDS-PAGE loading buffer (1:1). The mixtures were boiled at 95° C. for 10 min, followed by centrifugation at 15,000 rpm for 10 min. Samples were analyzed using 12% SDS-PAGE. The separated samples were blotted to a PVDF membrane and subjected to WB analysis using an antibody against the Prp8 intein. Green et al., “Spliceosomal Prp8 Intein at the Crossroads of Protein and RNA Splicing,” PLoS Biol 17(10):e3000104 (2019), which is hereby incorporated by reference in its entirety.

Mass spectrometry (MS)—The electrospray ionization mass spectrometry (ESI-MS) experiment was carried out as described. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. Briefly, the Cga Prp8 intein in pXI and pET28a backgrounds to yield intein only or intein flanked by extein sequence (30 μM) was incubated at 25° C. with compounds (300 μM) in a binding buffer (50 mM sodium phosphate, 100 mM NaCl, pH 7.0) for ˜18 h. Mass spectrometry of the incubated samples was carried out on an LTQ Orbitrap Velos instrument (Thermo Scientific, Grand Island, N.Y.) in the positive ion mode.

LC-MS/MS was carried out as described (Li et al., “Existing Drugs as Broad-Spectrum and Potent Inhibitors for Zika Virus by Targeting NS2B-NS3 Interaction,” Cell Research 27(8):1046-1064 (2017), which is hereby incorporated by reference in its entirety). Briefly, the protein band of interest on SDS-PAGE gel was manually excised, processed, and treated with 25 ng of sequencing grade modified trypsin (Sigma-Aldrich), as described (Li et al., “Existing Drugs as Broad-Spectrum and Potent Inhibitors for Zika Virus by Targeting NS2B-NS3 Interaction,” Cell Research 27(8):1046-1064 (2017), which is hereby incorporated by reference in its entirety). The digested peptides were extracted, separated, desalted, and subjected to LC-MS/MS analysis with a Jupiter C18 column (3 μm, 100 μm ID×150 mm, Phenomenex, Torrance, Calif.), using a QSTAR® XL Hybrid LC/MS/MS System, as described (Li et al., “Existing Drugs as Broad-Spectrum and Potent Inhibitors for Zika Virus by Targeting NS2B-NS3 Interaction,” Cell Research 27(8):1046-1064 (2017), which is hereby incorporated by reference in its entirety).

MS data acquisition was performed using Analyst QS 1.1 software (ABSciex) in positive ion mode for information dependent acquisition (IDA) analysis. The MASCOT 2.5 from Matrix Science (London, UK) was used to query fungal protein and contaminant data subsets using the following parameters: peptide mass tolerance, 0.3 Da; MS/MS ion mass tolerance, 0.3 Da; and allowing up to two missed cleavages. Several variable modifications were applied, including methionine oxidation and cysteine carbamidomethylation. Only significant scores for the peptides defined by the Mascot probability analysis (“Matrix Science Scoring Help,” which is hereby incorporated by reference in its entirety) greater than “identity” with 95% confidence were considered for the peptide identification.

Protein thermal shift assay—The protein thermal-shift assay (PTSA) was conducted using an Applied Biosystem 7500 Fast Real-Time PCR System (ThermoFisher Scientific) from 25 to 80° C., with methods similar to those described previously (Li et al., “Existing Drugs as Broad-Spectrum and Potent Inhibitors for Zika Virus by Targeting NS2B-NS3 Interaction,” Cell Research 27(8):1046-1064 (2017), which is hereby incorporated by reference in its entirety). Briefly, the Prp8 intein at a final concentration of 2.5 μM in 1×PBS was mixed with each compound to attain a 4.8 μM final concentration in 1.6% DMF for 30 min at 25° C. PTSA was carried out in the MicroAmp® Fast Optical 96-Well Reaction Plate (ThermoFisher Scientific). Thermal denaturation was monitored using SYPRO Orange (Life Technologies) according to manufacturer's manual. The melting temperature (Tₘ) was calculated, using a Derivative model using the Protein Thermal Shift™ Software v1.0 (ThermoFisher Scientific). Compounds were considered to be binders when ΔTₘ>0.5° C.

Surface plasmon resonance (SPR)—Surface plasmon resonance (SPR) was used to determine the affinity and kinetic analyses of the interactions between each drug and the Prp8 intein at 25° C. using a ProteOn XPR36 SPR instrument (Bio-Rad). The Prp8 intein and mutants were immobilized onto a ProteOn™ GLH sensor chip (2,500-7,000 RU) (Bio-Rad). A 3-fold dilution series of compounds was injected as the analytes. A blank surface blocked by ethanolamine was used as the control surface. The experiment was carried out at a flow rate of 100 μl/min using a PBSTD buffer containing 1×PBS, 0.005% surfactant P20, and 5% DMSO. Association (kₐ) and dissociation (k_{d}) rates, as well as the dissociation constant (K_{D}), were obtained by global fitting of the SPR data from multiple concentrations to a simple 1:1 Langmuir binding model, using the ProteOn Manager software suite (Bio-Rad).

Fungal susceptibility test—The fungal susceptibility test was carried out with a compound concentration series as described. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. The broth microdilution method was used to determine the compound MIC values against C. neoformans. In brief, serial 2-fold dilutions of the 6G-318SS (20 to 0.03 μg/ml) were made in 100 μl RPMI-1640 medium with MOPS in 96-well U-bottom plates. The C. neoformans H99 strain (100 μl of 0.5×10⁵/ml) in log phase was added to each well. After incubating the cells at 30° C. for 48 h, the cell culture was visualized to determine MIC. The MIC break-point was defined as no visible fungal growth by naked eye or by determining the 90% growth inhibition using absorbance at 630 nm, compared with no inhibitor control in a 96-well plate.

To determine minimum fungicidal concentration (MFC), the cultured cells in selected wells were first suspended by gentle pipetting. Then 50 ul of the cell culture suspension from each well was plated onto the Sabouraud dextrose agar (SDA) plates and further incubated for 48 h. MFC is defined as the minimum fungicidal concentration where there is no growth after plating on the SDA plates. The ratio of MFC/MIC≤4 is considered as fungicidal.

For resistance studies, C. neoformans H99 strain transformed with an empty pXL1-PTEF1 vector, the Prp8-pXL1-PTEF1 (WT Prp8) construct, or the AAAA mutant were used, as described. Li et al., “Cisplatin Protects Mice From Challenge of Cryptococcus Neoformans by Targeting the Prp8 Intein,” Emerging Microbes & Infections 8(1):895-908 (2019), which is hereby incorporated by reference in its entirety. MICs for selected compounds were determined for these transformed cells as described above.

Sensititre YeastOne Y09 AST assay—Fungal susceptibility to a combination of compounds was carried out using the Sensititre YeastOne Y09 AST assay kit (Thermofisher), according to the manufacturer's manual. Briefly, fungal C. neoformans cells in exponential growth phase were diluted to a final OD₅₃₀ of 0.1. Then 20 μl diluted cells were further diluted into 10 ml YPD as a stock. Prior to cell addition, a Sensititre YeastOne Y09 AST assay plate was prepared with 6G-318S (100 μl) at 2× final concentration (0.16 μg/ml) or DMF control in YPD. The stock C. neoformans (100 μl, ˜200 cells) were dispensed into the compound-ready 96-well plate. The fungal cells (treated or with DMF) were incubated at 30° C. for 48 h. The cell cultures were visualized by naked eye. The MIC break points were determined according to manufacturer's instructions.

Checkerboard assay—To determine the impact on potency of 6G-318S in combination with known antifungals, a checkerboard assay was carried by the microbroth dilution method in a 96-well plate as per Clinical and Laboratory Standards Institute (CLSI)-M60. A combination of 6G-318S (0.063-4.0 μg/ml) with amphotericin B (AmB, 0.005 to 4.62 μg/ml) or 5-fluorocytosine (5-FC, 0.032 to 32.4 μg/ml) or itraconazole (IZ, 0.003 to 2.820 μg/ml) or voriconazole (VOR, 0.0003 to 0.349 ug/ml) were used in 96-well flat bottom plates for absorbance reading and round bottom tissue culture plates for visual examination. Compounds were initially dissolved in DMSO and diluted in RPMI-1640-MOPS media four times the targeted final concentration and 50 μl was added per well. The second compound was similarly diluted and 50 μl was added per well, to a final volume of 100 μl/well. C. neoformans H99 inoculum (100 μl) prepared from freshly grown cultures and diluted to an absorbance of 0.1 at 530 nm in water and further diluted (1:500) in RPMI-1640-MOPS was added to each well. To determine the fungicidal concentration of a compound, the wells with no visible growth were serially diluted, plated on SDA, and incubated at 30° C. for 48-72 h for the recovery of colony-forming units. Additionally, the cell density of treated and untreated wells was also estimated by reading absorbance at 630 nm using an EL808 reader (Bio-Tek). Percentage of inhibition compared to that of the no-drug-control well was calculated. MIC is defined as the minimum concentration of drugs required to inhibit 90% of the growth. Fractional inhibitory concentration (FIC) index value is calculated as the ratio between MIC of compounds in combination and the sum of MIC of individual drug alone. The FIC index value is then used to categorize the interaction of the two compounds tested. FIC index value of ≤0.5 is considered as synergy, 0.5 to 1 is additive, >1 to 4 is indifferent, >4 is antagonistic. Doern, C., “When Does 2 plus 2 Equal 5?A Review of Antimicrobial Synergy Testing,” Journal of Clinical Microbiology 52(12):4124-4128 (2014), which is hereby incorporated by reference in its entirety.

Statistical analysis—All experiments were performed in triplicate unless specified otherwise. GFP fluorescence in gel was quantified using the Bio-Rad Gel Doc EZ system and Image Lab™ software #1709690 (Bio-Rad). One-way ANOVA and student T-test were used to carry out statistical analyses with the Prism software.

### Example 2—Development of Split Green Fluorescence Protein (GFP)-Based Prp8 Intein Splicing Assay

Previously, a high throughput screening (HTS) assay was developed based on split Renilla luciferase (RLuc-Prp8). Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. Although the RLuc-Prp8 assay is highly robust, it can potentially identify luciferase inhibitors. Although it is not surprising that compounds often inhibit multiple targets, some compounds may yield false positives by inhibiting luciferase instead of Prp8 intein splicing. One way to eliminate these false positives is to screen positive hits against luciferase itself. However, this method may potentially remove true inhibitors that display dual inhibition activities towards both Prp8 intein splicing and luciferase.

To better address this concern, an intein splicing assay with a split-GFP was developed, using methods similar to those described previously. Chan et al. “Exploring Intein Inhibition by Platinum Compounds as an Antimicrobial Strategy,” The Journal of Biological Chemistry 291(43):22661-22670 (2016) and Gangopadhyay et al., “An in Vitro Screening System for Protein Splicing Inhibitors Based on Green Fluorescent Protein as an Indicator,” Analytical Chemistry 75(10):2456-2462 (2003), both of which are hereby incorporated by reference in their entirety. The Prp8 intein was inserted between GFP residues G128 and D129 using a mega-PCR mutagenesis strategy (Li et al., “Cisplatin Protects Mice From Challenge of Cryptococcus neoformans by Targeting the Prp8 Intein,” Emerging Microbes & Infections 8(1):895-908 (2019); Chan et al. “Exploring Intein Inhibition by Platinum Compounds as an Antimicrobial Strategy,” The Journal of Biological Chemistry 291(43):22661-22670 (2016); and Brecher et al., “A Conformational Switch High-Throughput Screening Assay and Allosteric Inhibition of the Flavivirus NS2B-NS3 Protease,” PLoS Pathogens 13(5):e1006411 (2017), all of which are hereby incorporated by reference in their entirety). The GFP-Prp8 intein fusion protein (also referred to herein as GFP-Prp8i) was expressed as inclusion bodies, refolded at pH 9.0 to prevent auto-splicing, and purified using affinity and size exclusion chromatography. Li et al., “Cisplatin Protects Mice From Challenge of Cryptococcus neoformans by Targeting the Prp8 Intein,” Emerging Microbes & Infections 8(1):895-908 (2019) and Chan et al. “Exploring Intein Inhibition by Platinum Compounds as an Antimicrobial Strategy,” The Journal of Biological Chemistry 291(43):22661-22670 (2016), both of which are hereby incorporated by reference in their entirety.

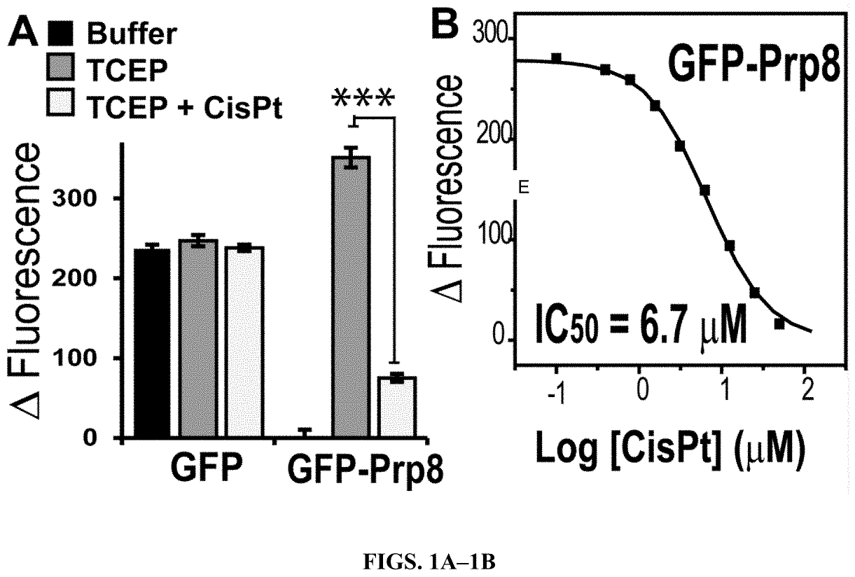

The intein splicing assay was initiated by diluting the purified, concentrated GFP-Prp8i into assay buffer at pH 8 in a 96-well plate. Reducing agents such as Tris (2-carboxyethyl) phosphine (TCEP) are required to reduce the nucleophilic cysteine (Cys1) and thereby trigger in vitro intein splicing. Li et al., “Cisplatin Protects Mice From Challenge of Cryptococcus Neoformans by Targeting the Prp8 Intein,” Emerging Microbes & Infections 8(1):895-908 (2019), which is hereby incorporated by reference in its entirety. As expected, purified GFP-Prp8i did not generate fluorescence in the absence of TCEP (FIG. 1A). In contrast, addition of TCEP led to a significant increase in fluorescence, presumably because TCEP triggers Prp8 intein splicing to generate reconstituted full-length GFP (FIG. 1A, buffer vs. TCEP). To rule out the possibility that TCEP may affect native GFP fluorescence, wild-type GFP fluorescence was measured with or without TCEP incubation. As shown in FIG. 1A, TCEP did not have any effect on the native GFP fluorescence.

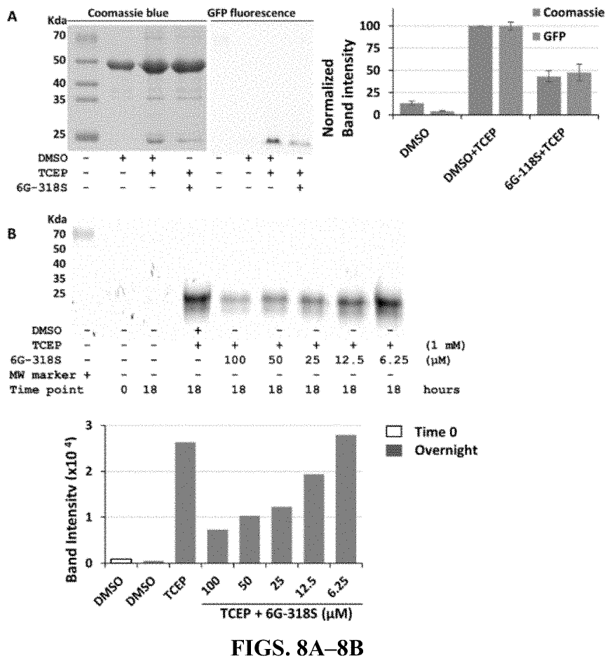

The Prp8 intein insertion site between G128 and D129 in GFP is not in a conserved region essential for GFP fluorophore formation. To eliminate the possibility that the observed fluorescence increase is due to unspliced GFP-Prp8i precursor refolding and maturation, SDS-PAGE analysis of GFP-Prp8i was performed in the presence and absence of TCEP (FIG. 8). The GFP-Prp8i precursor did not self-splice after overnight incubation in the absence of TCEP. Addition of TCEP triggered GFP-Prp8i splicing, resulting in an additional band near 25 KDa, which is consistent with the molecular weight of native full-length GFP. The GFP-Prp8i precursor did not become fluorescent regardless of TCEP addition (FIGS. 8A, 8B). In contrast, the 25-KDa protein showed significant fluorescence upon TCEP addition, indicating that the fluorescence increase observed in the 96-well plate resulted from Prp8 intein splicing.

### Example 3—Application of Split-GFP Assay for Small Molecule Screening

To further validate the split-GFP assay, cisplatin was used as a control inhibitor. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety). First, it was shown that addition of cisplatin did not affect native GFP fluorescence, (FIG. 1A). Notably, the fluorescence increase of the GFP-Prp8i fusion was significantly reduced by cisplatin (FIG. 1A). The assay had a Z′ score of 0.89, indicating that it is suitable for HTS (Z′>0.5) (Iversen et al. “HTS Assay Validation,” Assay Guidance Manual (Bethesda, Md.): Eli Lilly & Company and the National Center for Advancing Translational Sciences (2004), which is hereby incorporated by reference in its entirety) (FIG. 1A). Dose-dependent inhibition of Prp8 intein splicing by cisplatin was next investigated. This data showed that cisplatin inhibited Prp8 intein splicing in a dose-dependent manner, with an IC₅₀ of 6.7 μM (FIG. 1B). This IC₅₀ value is comparable to that determined using the RLuc-Prp8 assay (2.5 μM) (Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety).

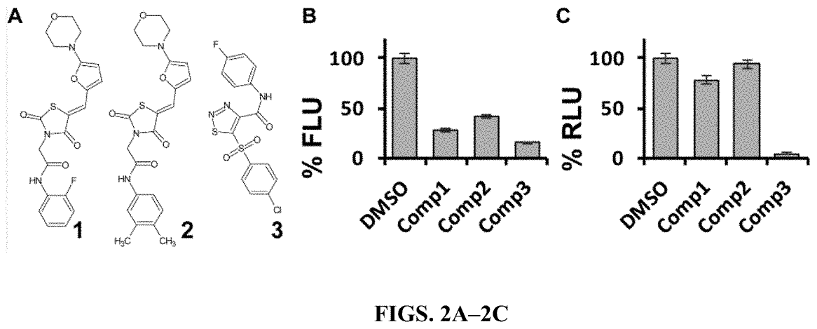

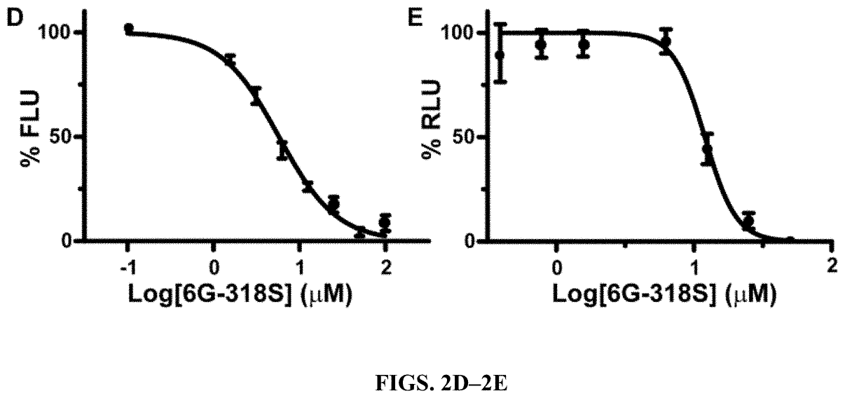

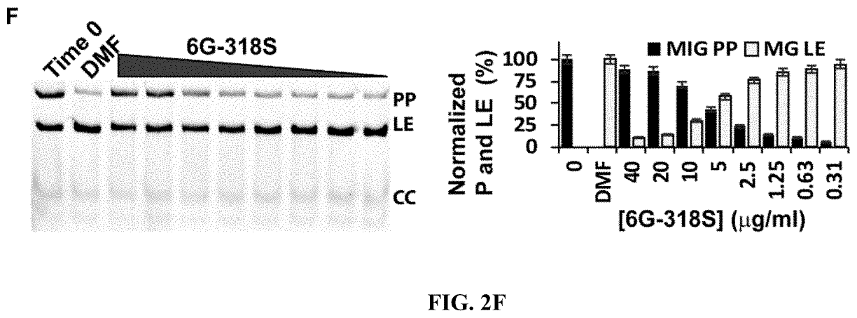

Next, a pilot screen of small molecule inhibitors of Prp8 intein splicing was carried out. The compound library (Timtec) contained about 240 putative protease inhibitors (Owen et al. “Forster Resonance Energy Transfer-Based Cholesterolysis Assay Identifies a Novel Hedgehog Inhibitor,” Analytical Biochemistry 488:1-5 (2015), which is hereby incorporated by reference in its entirety) and 60 compounds from an in-house collection. At 20 μM, three compounds showed greater than 50% inhibition of the GFP-Prp8i signal (FIGS. 2A, 2B). The three compounds were further tested using the RLuc-Prp8 intein splicing assay. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. These results showed that only compound 3, 5-[(4-chlorophenyl)sulfonyl]-N-(4-fluorophenyl)-1,2,3-thiadiazole-4-carboxamide (commercial name 6G-318S) suppressed more than 50% of RLuc-Prp8 intein splicing activity at 20 μM (FIG. 2C).

### Example 4—Inhibition of Prp8 Intein Splicing by 6G-318S

Using a concentration series of 6G-318S, these results indicated that 6G-318S inhibited Prp8 intein splicing in a dose-dependent manner in both GFP-Prp8i and RLuc-Prp8 assays. The IC₅₀ values determined by the two assays were 5.8 μM and 11.2 μM, respectively (FIGS. 2D, 2E). In addition, using in-gel GFP-fluorescence, it was shown that 6G-318S inhibited GFP-Prp8i splicing in a dose-dependent manner (FIG. 8B).

An additional fluorescence reporter system was used to investigate inhibition of Prp8 intein splicing by 6G-318S. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. This reporter system, MIG-Prp8, comprising a fusion protein of the Prp8 intein flanked by maltose binding protein (MBP) and green fluorescent protein (GFP), allowed observation of functional Prp8 intein splicing in Escherichia coli lysate. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. Incubation of the MIG-Prp8 fusion protein precursor (PP) with TCEP triggered Prp8 intein splicing, leading to reduced PP and accumulation of ligated extein (LE) product MBP-GFP (MG) (FIG. 2F, time 0 vs. solvent dimethylformamide (DMF) control). C-terminal cleavage product (CC; GFP) could also be visualized. Using the MIG-Prp8 intein construct, in vitro inhibition of Prp8 intein splicing by 6G-318S was shown, leading to accumulation of the MIG-Prp8 precursor PP and decrease of LE product in a dose-dependent manner (FIG. 2F). Quantification of the PP and LE bands indicated a perfectly inverse relationship (FIG. 2F, right panel). Overall, the data indicated that 6G-318S inhibits Prp8 intein splicing in vitro.

### Example 5—Derivatives of 6G-318S and Inhibition Selectivity

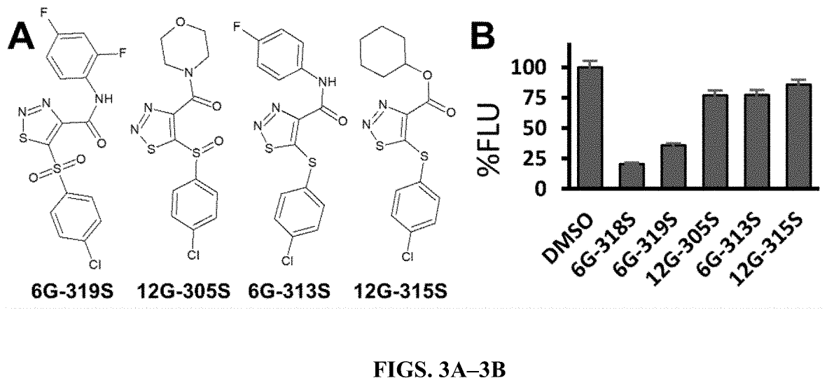

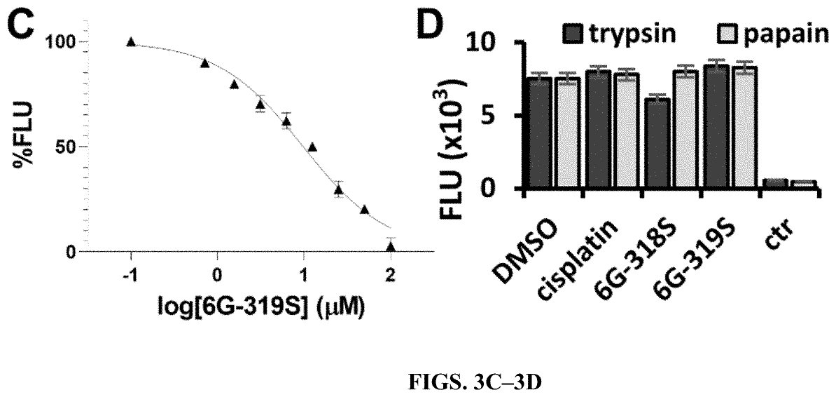

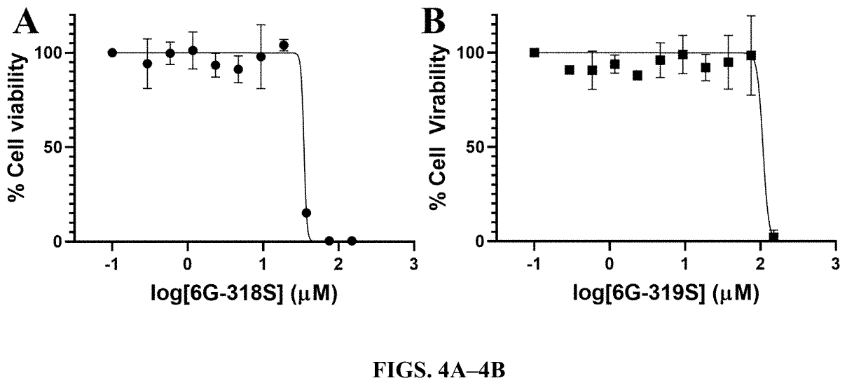

To explore 6G-318S analogs, a substructure search was performed and four commercially available derivatives were found, namely 6G-319S, 12G-305S, 6G-313S, and 12G-315S (FIG. 3A). The Prp8 intein splicing inhibition assay was performed using the GFP-Prp8i construct for these compounds at 20 μM concentration. The results showed that none of the four compounds displayed better inhibition than 6G-318S, but that one derivative, 6G-319S, showed more than 50% inhibition of the Prp8 intein splicing (FIG. 3B). In dose-response inhibition of the Prp8 intein splicing using the GFP-Prp8i construct, 6G-319S registered an IC₅₀ value of 9.7 μM (FIG. 3C), in the same range as 6G-318S (5.8 μM). Apparently, the aniline moiety and the phenylsulfone pharmacophore group are essential for these thiadiazole derivatives to maintain their inhibitory activities against Prp8 intein splicing. The other sulfide or sulfoxide analogs were found to be inactive.

Intein self-splicing may resemble serine/cysteine protease activity in function by using nucleophilic residues to cleave peptide bonds. The identified inhibitors, 6G-318S and 6G-319S, were further evaluated for specificity using a counter-screening assay measuring inhibition of representative serine (trypsin) and cysteine (papain) proteases, as was described previously. Li et al., “Existing Drugs as Broad-Spectrum and Potent Inhibitors for Zika Virus by Targeting NS2B-NS3 Interaction,” Cell Research 27(8):1046-1064 (2017) and Li et al., “Erythrosin B is a Potent and Broad-Spectrum Orthosteric Inhibitor of the Flavivirus NS2B-NS3 Protease,” Antiviral Research 150:217-225 (2018), both of which are hereby incorporated by reference in their entirety. Cisplatin was also included. Using a FRET substrate Abz-RRRRSAG-nTyr developed previously (Li et al., “Existing Drugs as Broad-Spectrum and Potent Inhibitors for Zika Virus by Targeting NS2B-NS3 Interaction,” Cell Research 27(8):1046-1064 (2017) and Li et al., “Erythrosin B is a Potent and Broad-Spectrum Orthosteric Inhibitor of the Flavivirus NS2B-NS3 Protease,” Antiviral Research 150:217-225 (2018), both of which are hereby incorporated by reference in their entirety), it was shown that at 400 μM, neither 6G-318S, 6G-319S, nor cisplatin significantly inhibited the protease activities of trypsin and papain (FIG. 3D). In contrast, control inhibitor antipain dihydrochloride (AP-2HCl) (Suda et al., “Antipain, A New Protease Inhibitor Isolated From Actinomycetes,” The Journal of Antibiotics (Tokyo) 25(4):263-266 (1972), which is hereby incorporated by reference in its entirety) at 3.5 μM completely abolished the protease activities (FIG. 3D). The results suggest that these compounds are specific to Prp8 intein splicing.

### Example 6—Inhibition of Intein Splicing and Growth of C. neoformans

Using a broth microdilution assay (Rex et al., “Reference Method for Broth Dilution Antifungal Susceptibility Testing of Yeasts,” 3rd Edition (Cold Spring Harbor Laboratory) 3rd Ed p 40 (2008), which is hereby incorporated by reference in its entirety), the antifungal efficacy of 6G-318S and 6G-319S was evaluated against the Prp8 intein-containing fungus C. neoformans H99 strain. C. albicans, a fungus without the Prp8 intein, was also used as a control. These results demonstrated that the compounds 6G-318S and 6G-319S are potent inhibitors of C. neoformans (Table 1).

TABLE 1
Inhibition of Pathogenic Fungi by Identified
Compounds Using Microdilution Assay

| Com- | MIC (μg/mL) | | CC₅₀ (μg/mL)/(μM) | TI |
|---|---|---|---|---|
| pounds | C. neoformans | Candida albicans | A549 | Cne |
| 6G-318S | 0.62 | 5.0 | 13.9 (35) | 22 |
| 6G-319S | 1.3 | 20.8 | 44.9 (108) | 35 |

TI, therapeutic index.

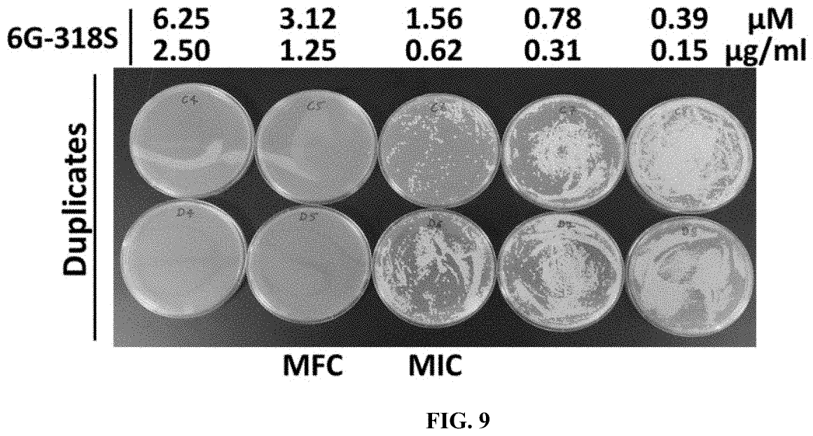

The minimum inhibitory concentration (MIC) values (0.6 μg/ml and 1.3 μg/ml for 6G-318S and 6G-319S, respectively) (Table 1) were comparable to or better than those of the current first-line antifungal drugs, such as 5-fluorocytosine (5-FC), itraconazole (IZ), fluconazole (FZ), and amphotericin B (AmB). Archibald et al., “Antifungal Susceptibilities of Cryptococcus neoformans,” Emerging Infectious Disease 10(1):143-145 (2004), which is hereby incorporated by reference in its entirety. In contrast, 6G-318S and 6G-319S did not inhibit growth of C. albicans (MIC>5 μg/ml), (Table 1). These results suggest that these compounds selectively inhibit the growth of Prp8 intein-containing fungi. The minimum fungicidal concentration (MFC) of 6G-318S was found to be 1.25 μg/ml for C. neoformans (FIG. 9). Compounds exhibiting a MFC/MIC ratio <4 are considered fungicidal, and 6G-318S displays a ratio of 2, thus confirming that it is fungicidal. Hazen, K. C., “Fungicidal Versus Fungistatic Activity of Terbinafine and Itraconazole: An In Vitro Comparison,” Journal of the American Academy of Dermatology 38 (5 Pt 3):S37-41 (1998), which is hereby incorporated by reference in its entirety.

Compound 6G-319S differs from 6G-318S only by the addition of a fluoride to the phenyl ring (FIGS. 2A and 3A). Both in vitro Prp8 intein splicing and in vitro fungal killing assays indicated that the introduction of a 2-F substituent on the aniline moiety (as with 6G-319S) led to a slight loss of potency (FIGS. 2D and 3C, Table 1). However, this modification significantly improved its cytotoxicity profile (FIG. 4). As shown in Table 1, 6G-319S showed a 3.2-fold higher cytotoxicity value (CC₅₀) than 6G-318S (CC₅₀: compound concentration required for 50% inhibition of cell viability). Values were 44.9 μg/ml (˜108 μM) for 6G-319 and 13.9 μg/ml (˜35 μM) for 6G-318S, indicating that 6G-319S is less toxic to human cells than 6G-318S. The results also demonstrated that these compounds have a good (>10) therapeutic index (TI) because the cytotoxicity CC₅₀ values of compounds 6G-318S and 6G-319S are 22- and 35-fold, respectively, of their corresponding MIC values towards C. neoformans.

### Example 7—Synergy with Existing Drugs

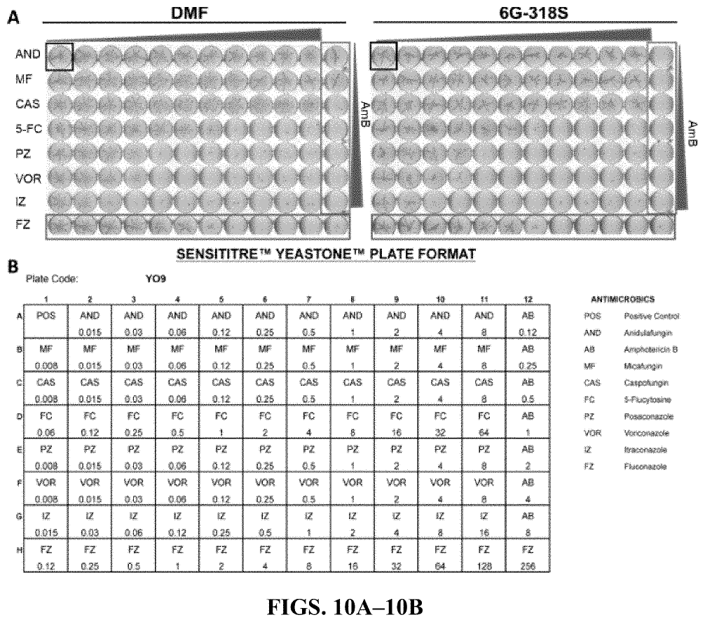

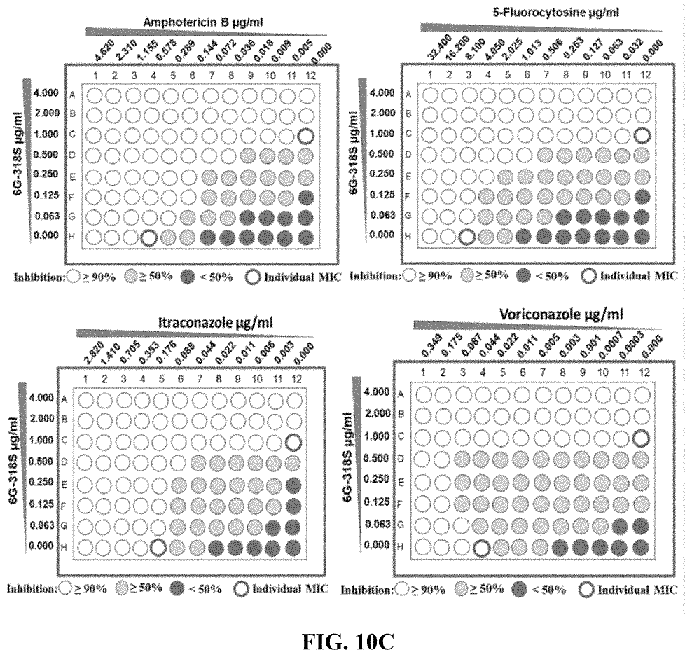

To test if the Prp8 intein splicing inhibitor 6G-318S has synergistic effects with clinically used drugs, a drug combination study was first performed using the Sensititre YeastOne Y09 AST Kit (Thermofisher) that contains 9 drugs, AmB, 5-FC, IZ, FZ, Anidulafungin (AND), Micafungin (MF), Caspofungin (CAS), Posaconazole (PZ), and Voriconazole (VOR). 6G-318S was chosen at 0.16 ug/ml, at which concentration C. neoformans is viable. As shown in FIGS. 10A, 10B, addition of 6G-318S did not change the MICs for AND, MF, CAS, PZ and FZ. In contrast, 6G-318S addition reduced MICs more than 4-fold for AmB and 2-fold for 5-FC, IZ and VOR. It is noted that combination of AmB at the lowest available concentration in the test plate with 6G-318S was sufficient to suppress C. neoformans growth (FIG. 10A). Therefore, the AmB MIC reduction may be higher than 4-fold when combined with 6G-318S.

To further investigate a possible synergistic effect of 6G-318S with existing drugs, the checkerboard assay (Fothergill, A., “Antifungal Susceptibility Testing: Clinical Laboratory and Standard Institute (CLSI) Methods,” Interactions of Yeasts, Moulds and Antifungal Agents: How to Detect resistance, Hall G. 65-75 (2012); Hai et al., “The Combination of Tamoxifen With Amphotericin B, But Not With Fluconazole, Has Synergistic Activity Against the Majority of Clinical Isolates of Cryptococcus neoformans,” Mycoses 62(9):818-825 (2019); and Lewis et al., “Comparison of Etest, Chequerboard Dilution and Time-Kill Studies for the Detection of Synergy or Antagonism Between Antifungal Agents Tested Against Candida Species,” The Journal of Antimicrobial Chemotherapy 49(2):345-351 (2002), all of which are hereby incorporated by reference in their entirety) was carried out to determine the fractional inhibitory concentration (FIC) (FIG. 10C, Table 2).

TABLE 2
Effect of 6G318S in Combination With Known Antifungals

| Well | Com- | MIC-alone | MIC-comb | FIC | |
|---|---|---|---|---|---|
| position | pounds | (μg/mL)* | (μg/mL)* | index* | Effect |
| F6 | 6G-318S | 1.000 | 0.125 | 0.37 | Synergistic |
| | AmB | 0.578 | 0.144 | | |
| D6 | 6G-318S | 1.000 | 0.500 | 0.63 | Additive |
| | 5-FC | 8.100 | 2.025 | | |
| D6 | 6G-318S | 1.000 | 0.500 | 1.00 | Additive |
| | IZ | 0.176 | 0.088 | | |
| G3 | 6G-318S | 1.000 | 0.063 | 2.04 | Indifferent |
| | Vor | 0.044 | 0.087 | | |

*MIC and FIX Index were calculated by checkerboard assay, as illustrated in FIGS. 10A-10C. FIC index value of ≤0.5 is considered as synergy, >0.5 to 1 is additive, >1 to 4 is indifferent.

In the FIC experiment, 6G-318G showed a synergistic effect (FIC 0-0.5) with AmB with a FIC index value of 0.37. Doern, C., “When Does 2 plus 2 Equal 5? A Review of Antimicrobial Synergy Testing,” Journal of Clinical Microbiology 52(12):4124-4128 (2014), which is hereby incorporated by reference in its entirety. The combination of 6G-318G with 5-FC or IZ had an additive effect (FIC 0.5-1), with FIC indices of 0.63 and 1, respectively. The combination of 6G-318G and VOR had an indifferent effect (FIC 1-4), with a FIC index of 2.04.

### Example 8—Inhibitors Bind Covalently to the Active Site of the Prp8 Intein

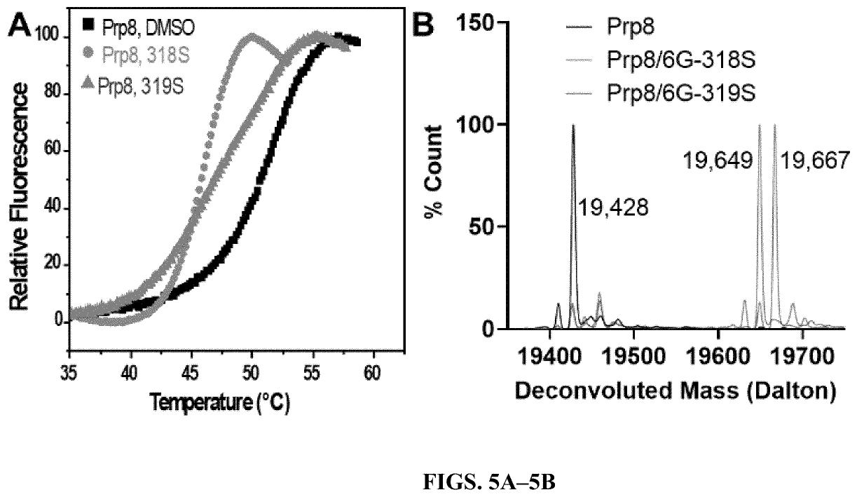

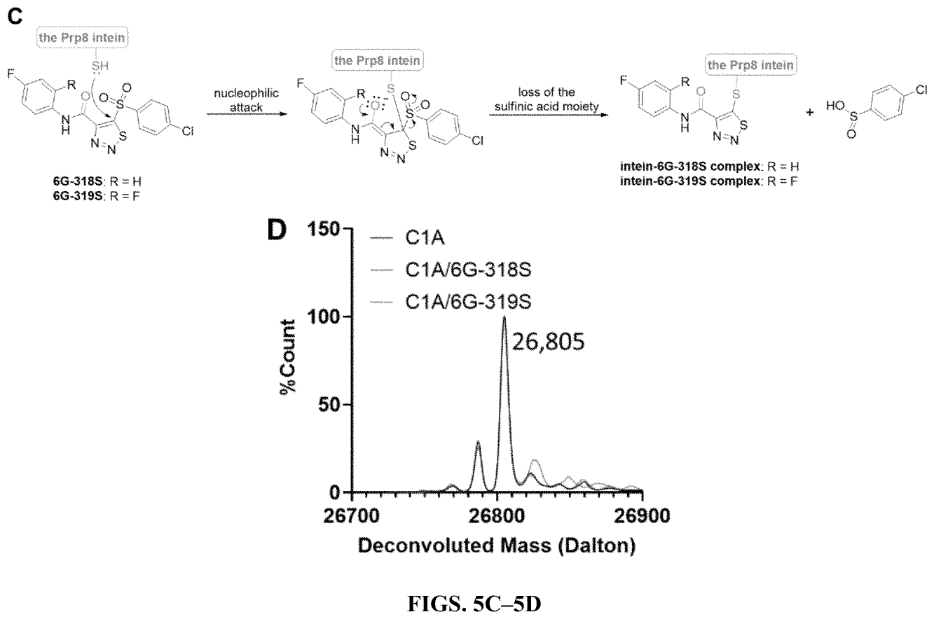

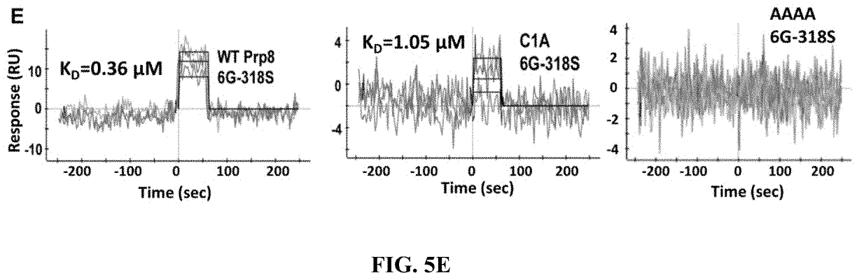

To investigate the mode of action, it was first evaluated if the compounds bind to the Prp8 intein using a protein thermal shift assay (PTSA), as described previously. Brecher et al., “A Conformational Switch High-Throughput Screening Assay and Allosteric Inhibition of the Flavivirus NS2B-NS3 Protease,” PLoS Pathogens 13(5):e1006411 (2017), which is hereby incorporated by reference in its entirety. Results of the PTSA confirmed that addition of the compounds induced large Tₘ changes (FIG. 5A), indicating binding of the small molecule inhibitor to the Prp8 intein. Interestingly, the binding of intein inhibitors decreased Tₘ of the Prp8 intein, indicating reduced protein stability. Covalent modification of active site residues by inhibitors could account for the reduced protein stability (as described infra).

To further investigate the nature of inhibitor binding, electrospray ionization-mass spectrometry (ESI-MS) was performed, using purified Cga Prp8 intein as described previously. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. The Cga Prp8 intein only contains the intein residues without any flanking extein sequence, and is 88% identical to the Cne Prp8 intein. The Prp8 intein was incubated overnight with or without 6G-318S or 6G-319S and then subjected to ESI-MS analysis. As shown in FIG. 5B, deconvolution of the ESI-MS data revealed that untreated Prp8 intein had a molecular mass of 19,428 Dalton (Da), exactly as predicted from its sequence. In contrast, the deconvoluted molecular masses for the Prp8 samples treated with 6G-318S and 6G-319S, respectively, shifted 221 Da and 239 Da, compared to that of untreated Prp8 intein, to 19,649 and 19,667, respectively. The mass differences were much smaller than the molecular weights for 6G-318S (397.83 Da) and 6G-319S (415.82 Da). These results suggest that the compounds react covalently with the Prp8 intein, possibly through the active site Cys1 thiol, and become truncated in the process.

The mass difference between the adducts of compounds 6G-318S and 6G-319S upon binding to the Prp8 intein is 18 Da, which is the exact mass of the extra fluoride atom for 6G-319S compared to 6G-318S. The results indicated that the two Prp8 intein inhibitors 6G-318S and 6G-319S followed the same chemistry to react with the Prp8 intein. Examination of the chemical structures of 6G-318S and 6G-319S suggested that the free thiol group of the catalytic Cys1 of the Prp8 intein likely attacks the carbon-carbon double bond of the thiadiazole moiety of the inhibitors, resulting in the cleavage of the bond between the phenylsulfonyl and thiadiazole ring (FIG. 5C). This would lead to a phenylcarbamoyl-thiadiazole adduct of 223.2 Da (6G-318S) or 241.2 Da (6G-319S) to form the intein-inhibitor complex, followed by the removal of the 4-chlorobenzenesulfinic acid fragment (176.6 Da), according to the mass spectrometry analysis. Covalent reaction of the thiadiazole ring of the adduct with the active site Cys1 led to the loss of two hydrogens, resulting in a final total mass addition of 221 Da for 6G-318S or of 239 Da for 6G-319S to the Prp8 intein, respectively.

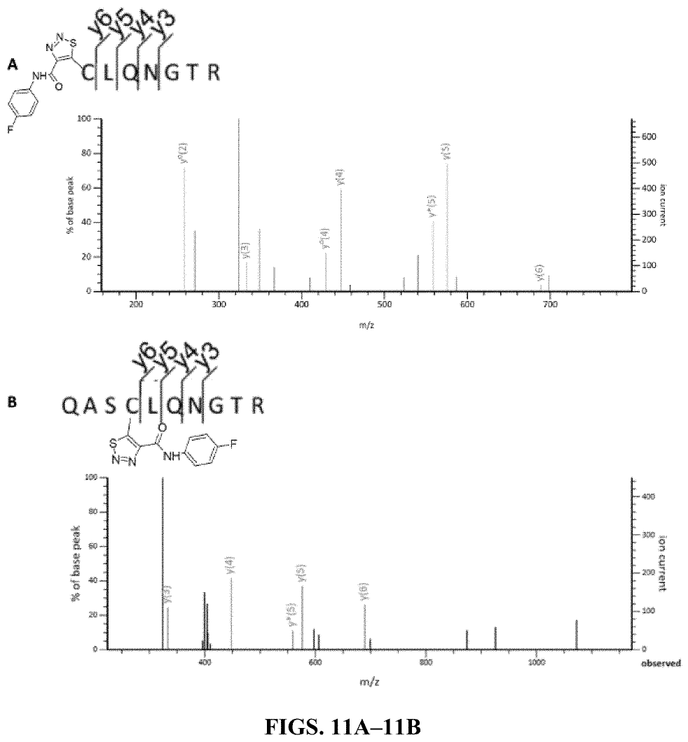

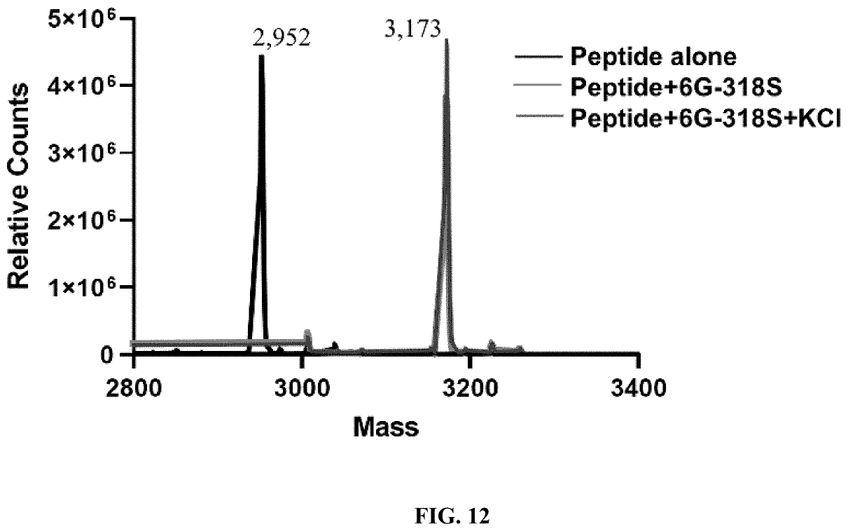

To verify this hypothesis, it was first evaluated if a Prp8 active -ite cysteine splicing-defective mutant (C1A) retained reactivity to the compounds. Using ESI-MS, it was shown that the compounds failed to modify the C1A mutant (FIG. 5D), because no mass shifts were observed for the complexes, which is in contrast to those of the wild-type (WT) Prp8 intein (FIG. 5B). Moreover, liquid chromatography with tandem mass spectrometry (LC-MS/MS) verified the predicted covalent modification of Cys1 of the Prp8 intein and a Cys-containing peptide by the 6G-318S fragment, both undergoing a mass shift of 221 Da, which is not affected by the choice of salt (FIGS. 11A, 12).

The Cga Prp8 intein used in the above experiments does not have extein residues flanking the intein due to the purification chemistry. Using an extein-containing Prp8 intein construct (Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety), it was shown that a Prp8 intein with exteins was modified at the Cys1 position by 6G-318, in the same way as the Prp8 intein without exteins (FIG. 11B).

To quantitate the molecular interaction between the splicing inhibitors and the Prp8 intein, a surface plasmon resonance (SPR) experiment was carried out to determine the binding affinity between 6G-318S and the Prp8 intein (FIGS. 5C and 5E). The results demonstrated that 6G-318S binds directly to the Prp8 intein with a binding affinity of 0.36 μM.

Previously, it was shown that the Prp8 intein splicing inhibitor cisplatin binds to the active site of the Prp8 intein. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. Three inactive mutants involving active-site residues were also generated, C1A, H169A, and C1A/H62A/D95A/H169A (the AAAA mutant). Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. To further explore the mechanism of action, the binding affinity of these mutants to 6G-318S was evaluated. As shown (FIG. 5E, Table 3), mutation of the C-terminal active site residue H169 to alanine did not affect the binding affinity. In contrast, mutation of the N-terminal active site residue Cys1 to alanine reduced the binding affinity nearly 3-fold. Moreover, the AAAA mutant completely lost binding affinity to 6G-318S, indicating that the compound requires an additional intein feature for binding (FIG. 5E, Table 3). The SPR data were also consistent with the MS results showing that 6G-318S reacted with the free thiol group of the Prp8 intein.

TABLE 3
Binding Affinity Between
6G-318S and the WT and Mutant Prp8 Inteins

| | WT | C1A | H169A | AAAA |
|---|---|---|---|---|
| K_{D} (μM) | 0.36 | 1.05 | 0.23 | ND |

ND, Not detected. Values were determined by SPR (FIG. 5E).

### Example 9—Inhibition of Prp8 Intein Splicing in C. neoformans

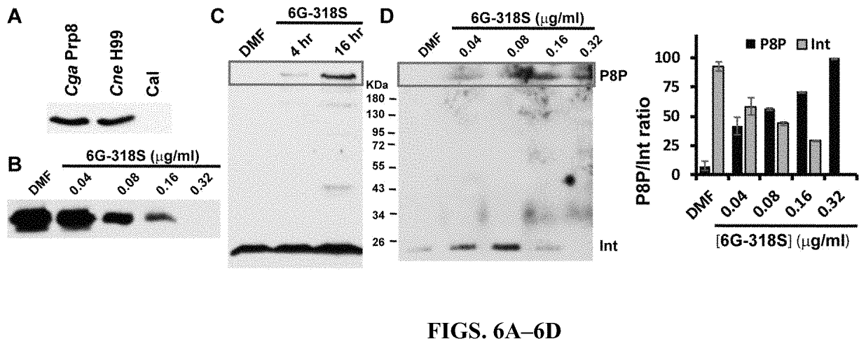

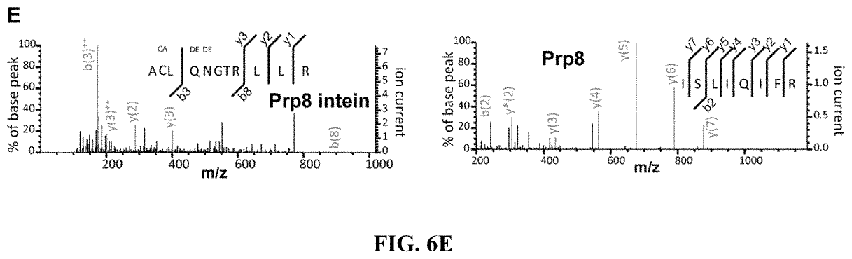

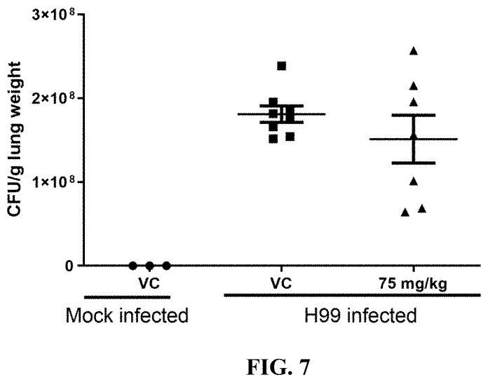

Using an anti-Prp8 intein antibody against the recombinant Prp8 intein, Western blot (WB) analysis (FIG. 6) was performed. The recombinant Cga Prp8 intein comprising of 170 amino acids was used as a positive control. It was first confirmed that a protein of 20 KDa could be detected by immunoblot (FIG. 6A). The antibody serum could also detect intein from the cell lysate of C. neoformans H99. In contrast, no band could be detected for the cell lysate of inteinless C. albicans, implying that the antibody was specific for the Prp8 intein. Next, WB analysis of cryptococcal samples was performed with/without 6G-318S at different concentrations at 3 h post-treatment (FIG. 6B). It was found that 6G-318S inhibited Prp8 splicing in vivo in C. neoformans, leading to a dose-dependent reduction of spliced Prp8 intein product (FIG. 6B). At 0.32 μg/ml, 6G-318S completely inhibited Prp8 splicing. The results were consistent with the fungal susceptibility studies, indicating that the MIC value for 6G-318S for the C. neoformans H99 strain was 0.62 μg/ml (Table 1).

A light band corresponding to a protein with a size larger than 180 KDa was observed for the C. neoformans H99 cell lysate treated with 6G-318S for 4 h after WB with the Prp8 intein antibody serum (FIG. 6C). In contrast, this putative precursor band (˜292 KDa) was not observed for the DMF-treated control sample. Next, WB analysis of the C. neoformans samples treated with different concentrations of 6G-318S or DMF control for 18 h was performed. As shown in FIG. 6D, even after this long incubation period, samples treated with DMF did not show high molecular-weight protein using the anti-Prp8 intein serum in WB. In contrast, a dose-dependent increase of high molecular-weight protein was seen for samples treated with 6G-318S. Conversely, as the high molecular-weight protein accumulated, the spliced Prp8 intein was diminished. Overall, a clear inverse relationship was observed between the accumulation of the high molecular-weight protein and the decrease of the spliced Prp8 intein in vivo for samples treated with 6G-318S (FIG. 6D, right panel).

It is suspected that the high molecular-weight protein represents unprocessed Prp8 protein precursor or branched intermediate (Liu et al., “Structure of the Branched Intermediate in Protein Splicing,” Proceedings of the National Academy of Sciences of the United States of America 111(23):8422-8427 (2014), which is hereby incorporated by reference in its entirety), with a molecular weight of 291.9 KDa, which accumulate over time due to splicing inhibition by 6G-318S. To test this hypothesis, the band was excised and MS proteomic analysis was carried out. The MS results verified that the high molecular weight protein contained representative peptides from the Cne Prp8 precursor, including both the Prp8 intein and exteins (FIG. 6E). These results are consistent with the hypothesis that inhibitors preventing intein splicing lead to accumulation of unprocessed and nonfunctional precursor, ultimately resulting in fungal death.

### Example 10—Overexpression of the Prp8 Intein in C. neoformans Leads to Drug Resistance

To further investigate the mechanism of inhibition in C. neoformans, an overexpression experiment was performed. A system was previously developed to overexpress wild-type (WT) and a non-splicing AAAA Prp8 intein mutant in cryptococcal cells under the strong TEF1 promoter. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. The empty vector, WT, and the AAAA mutant plasmid were individually transformed into the C. neoformans H99 strain. It was shown that transformation of the empty vector, Prp8 WT intein and the AAAA mutant did not affect the growth of cryptococcal cells in the absence of an inhibitor. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. Using this system, the MIC values of 6G-318S in these transformed cells were determined. The results showed that 6G-318S had identical MIC values with empty vector or AAAA mutant Prp8 intein-transformed H99 cells. In contrast, transformation of the WT Prp8 intein into H99 led to 4-fold increase of MIC for 6G-318S, compared to H99 transformed with empty vector or the AAAA mutant. Importantly, the result was consistent with the hypothesis that the Prp8 intein is the intracellular target of 6G-318S.

### Example 11—Discussion of Examples 2-10

C. neoformans and C. gattii cause cryptococcal meningitis (CM) and pulmonary cryptococcosis, which are very difficult to treat. There are over 1 million annual cases of CM worldwide, with estimated deaths of 700,000 per year. Perfect, John R. “Fungal Diagnosis: How Do We Do It and Can We Do Better?” Current Medical Research and Opinion 29 Suppl 4:3-11 (2013); Perfect, J. & Bicanic, T., “Cryptococcosis Diagnosis and Treatment: What Do We Know Now,” Fungal Genetics and Biology 78:49-54 (2015); Chen et al., “The Cryptococcus neoformans Transcriptome at the Site of Human Meningitis,” MBio 5(1):e01087-01013 (2014); and Zhai et al., “The Antidepressant Sertraline Provides a Promising Therapeutic Option for Neurotropic Cryptococcal Infections,” Antimicrobial Agents and Chemotherapy 56(7):3758-3766 (2012), all of which are hereby incorporated by reference in their entirety. CM is seen most commonly in immunocompromised patients, such as those with HIV, severe combined immunodeficiency, or post-organ transplant status. Brouwer et al., “Combination Antifungal Therapies for HIV-Associated Cryptococcal Meningitis: A Randomised Trial,” Lancet 363(9423):1764-1767 (2004) and Husain et al., “Cryptococcus neoformans Infection in Organ Transplant Recipients: Variables Influencing Clinical Characteristics and Outcome,” Emerging Infectious Disease 7(3):375-381 (2001), both of which are hereby incorporated by reference in their entirety. However, recent reports of infections caused by C. gattii in non-immunocompromised people have raised concerns about the overall threat of the Cryptococcus species to public health. MacDougall et al., “Spread of Cryptococcus gattii in British Columbia, Canada, and Detection in the Pacific Northwest, USA,” Emerging Infectious Disease 13(1):42-50 (2007); Fyfe et al., “Cryptococcus gattii Infections on Vancouver Island, British Columbia, Canada: Emergence of a Tropical Fungus in a Temperate Environment,” Canada Communicable Disease Report 34(6):1-12 (2008); Kronstad et al., “Expanding Fungal Pathogenesis: Cryptococcus Breaks Out of the Opportunistic Box,” Nature Reviews. Microbiology 9(3):193-203 (2011); Bartlett et al., “A Decade of Experience: Cryptococcus gattii in British Columbia,” Mycopathologia 173(5-6):311-319 (2012); Bartlett et al., “The Emergence of Cryptococcus gattii in British Columbia and the Pacific Northwest,” Current Infectious Disease Report 10(1):58-65 (2008); Upton et al., “First Contemporary Case of Human Infection with Cryptococcus gattii in Puget Sound: Evidence for Spread of the Vancouver Island Outbreak,” Journal of Clinical Microbiology 45(9):3086-3088 (2007); Byrnes et al., “A Diverse Population of Cryptococcus gattii Molecular Type VGIII in Southern Californian HIV/AIDS Patients,” PLoS Pathogens 7(9):e1002205 (2011); Byrnes et al., “Cryptococcus gattii: An Emerging Fungal Pathogen Infecting Humans and Animals,” Microbes and Infection 13(11):895-907 (2011); Byrnes et al., “Emergence and Pathogenicity of Highly Virulent Cryptococcus gattii Genotypes in the Northwest United States,” PLoS Pathogens 6(4):e1000850 (2010); Byrnes, E. & Heitman, J., “Cryptococcus gattii Outbreak Expands into the Northwestern United States with Fatal Consequences,” F1000 Biol Rep 1 (2009); Datta et al., “Spread of Cryptococcus gattii into Pacific Northwest Region of the United States,” Emerging Infectious Disease 15(8):1185-1191 (2009); and Byrnes et al. “First Reported Case of Cryptococcus gattii in the Southeastern USA: Implications for Travel-Associated Acquisition of an Emerging Pathogen,” PLoS One 4(6):e5851 (2009), all of which are hereby incorporated by reference in their entirety.

Cryptococcal infection is usually treated by combination therapy, with administration time ranging from 12-14 weeks to lifelong. Perfect et al., “Clinical Practice Guidelines for the Management of Cryptococcal Disease: 2010 Update by the Infectious Diseases Society of America,” Clinical Infectious Disease 50(3):291-322 (2010); Boulware et al., “Timing of Antiretroviral Therapy After Diagnosis of Cryptococcal Meningitis,” The New England Journal of Medicine 370(26):2487-2498 (2014); Rajasingham et al., “Cryptococcal Meningitis Treatment Strategies in Resource-Limited Settings: A Cost-Effectiveness Analysis,” PLoS Medicine 9(9):e1001316 (2012); Perea et al., “Antifungal Resistance in Pathogenic Fungi,” Clinical Infectious Diseases 35(9):1073-1080 (2002); Zuger et al., “Cryptococcal Disease in Patients with the Acquired Immunodeficiency Syndrome. Diagnostic Features and Outcome of Treatment,” Annals of Internal Medicine 104(2):234-240 (1986); and Boelaert et al., “Relapsing Meningitis Caused by Persistent Cryptococcal Antigens and Immune Reconstitution After the Initiation of Highly Active Antiretroviral Therapy,” AIDS 18(8):1223-1224 (2004), all of which are hereby incorporated by reference in their entirety. Mainstay drugs for CM include AmB, 5-FC, triazoles (fluconazole, voriconazole, posaconazole, itraconazole), and caspofungin (in combination therapy only). Brouwer et al., “Combination Antifungal Therapies for HIV-Associated Cryptococcal Meningitis: A Randomised Trial,” Lancet 363(9423):1764-1767 (2004); Perfect et al., “Clinical Practice Guidelines for the Management of Cryptococcal Disease: 2010 Update by the Infectious Diseases Society of America,” Clinical Infectious Disease 50(3):291-322 (2010); Boulware et al., “Timing of Antiretroviral Therapy After Diagnosis of Cryptococcal Meningitis,” The New England Journal of Medicine 370(26):2487-2498 (2014); Rajasingham et al., “Cryptococcal Meningitis Treatment Strategies in Resource-Limited Settings: A Cost-Effectiveness Analysis,” PLoS Medicine 9(9):e1001316 (2012); Cornely et al., “Liposomal Amphotericin B as Initial Therapy for Invasive Mold Infection: A Randomized Trial Comparing a High-Loading Dose Regimen with Standard Dosing (AmBiLoad trial),” Clinical Infectious Diseases 44(10):1289-1297 (2007); and Schiller, D. & Fung, H., “Posaconazole: An Extended-Spectrum Triazole Antifungal Agent,” Clinical Therapeutics 29(9):1862-1886 (2007), all of which are hereby incorporated by reference in their entirety. These drugs kill fungi through various mechanisms, including interfering with membrane permeabilisation (AmB) (Baginski, M. & Czub, J., “Amphotericin B and Its New Derivatives—Mode of Action,” Current Drug Metabolism 10(5):459-469 (2009) and Gray et al., “Amphotericin Primarily Kills Yeast by Simply Binding Ergosterol,” Proceedings of the National Academy of Sciences of the United States of America 109(7):2234-2239 (2012), both of which are hereby incorporated by reference in their entirety), inhibition of 14α-demethylase (triazoles) (Hitchcock et al., “Interaction of Azole Antifungal Antibiotics with Cytochrome P-450-Dependent 14 Alpha-Sterol Demethylase Purified From Candida albicans,” The Biochemical Journal 266(2):475-480 (1990), which is hereby incorporated by reference in its entirety), inhibition of DNA/RNA synthesis (5-FC) (Polak, A. & Scholer, H. J., “Mode of Action of 5-Fluorocytosine and Mechanisms of Resistance,” Chemotherapy 21(3-4):113-130 (1975), which is hereby incorporated by reference in its entirety), and inhibition of (1→3)-β-D-glucan synthase (caspofungin) (Deresinski, S. & Stevens, D., “Caspofungin,” Clinical Infectious Diseases 36(11):1445-1457 (2003), which is hereby incorporated by reference in its entirety). However, caspofungin does not work for C. neoformans (Chen et al., “Echinocandin Antifungal Drugs in Fungal Infections: A Comparison,” Drugs 71(1):11-41 (2011); Kartsonis et al., “Caspofungin: The First in a New Class of Antifungal Agents,” Drug Resistance Updates 6(4):197-218 (2003); Maligie, M. A. & Selitrennikoff, C., “Cryptococcus neoformans Resistance to Echinocandins: (1,3)beta-glucan Synthase Activity is Sensitive to Echinocandins,” Antimicrobial Agents and Chemotherapy 49(7):2851-2856 (2005); and Dupont, B. & Pialoux, G., “Amphotericin Versus Fluconazole in Cryptococcal Meningitis,” The New England Journal of Medicine 326(23):1568-1569 (1992), all of which are hereby incorporated by reference in their entirety). Despite the availability of these antifungal drugs, mortality rates associated with cryptococcal infections often exceed 50%. In addition, these drugs are frequently associated with severe side effects, high toxicity, and many serious drug-drug interactions. Gubbins et al., DRUG INTERACTIONS IN INFECTIOUS DISEASES (Humana Press, Totowa, N.J.) (2001); Moen et al., “Liposomal Amphotericin B: A Review of its Use as Empirical Therapy in Febrile Neutropenia and in the Treatment of Invasive Fungal Infections,” Drugs 69(3):361-392 (2009); and Kauffman, C., “Fungal Infections,” Proceedings of the American Thoracic Society 3(1):35-40 (2006), all of which are hereby incorporated by reference in their entirety. Moreover, because of the lengthy treatment, drug resistance is a significant problem. Pfaller, Michael A. “Antifungal Drug Resistance: Mechanisms, Epidemiology, and Consequences for Treatment,” The American Journal of Medicine 125 (1 Suppl):S3-13 (2012) and Ghannoum, M. A. & Rice, L. B. “Antifungal Agents: Mode of Action, Mechanisms of Resistance, and Correlation of These Mechanisms with Bacterial Resistance,” Clinical Microbiology Reviews 12(4):501-517 (1999), both of which are hereby incorporated by reference in their entirety.

The pathogenic cryptococcal fungi C. neoformans and C. gattii contain the Prp8 intein (Butler et al., “A Nuclear-Encoded Intein in the Fungal Pathogen Cryptococcus neoformans,” Yeast 18(15):1365-1370 (2001); Butler, M. & Poulter, R., “The PRP8 Inteins in Cryptococcus are a Source of Phylogenetic and Epidemiological Information,” Fungal Genetics and Biology 42(5):452-463 (2005); Liu, X. & Yang, J., “Prp8 Intein in Fungal Pathogens: Target for Potential Antifungal Drugs,” FEBS Letters 572(1-3):46-50 (2004); and Pearl et al., “Sequence Requirements for Splicing by the Cne PRP8 Intein,” FEBS Letters 581(16):3000-3004 (2007), all of which are hereby incorporated by reference in their entirety), which belong to the class 1 intein family (Butler et al., “A Nuclear-Encoded Intein in the Fungal Pathogen Cryptococcus neoformans,” Yeast 18(15):1365-1370 (2001); Liu, X. & Yang, J., “Prp8 Intein in Fungal Pathogens: Target for Potential Antifungal Drugs,” FEBS Letters 572(1-3):46-50 (2004); and Pearl et al., “Sequence Requirements for Splicing by the Cne PRP8 Intein,” FEBS Letters 581(16):3000-3004 (2007), all of which are hereby incorporated by reference in their entirety. The Prp8 protein is essential and the most highly conserved protein of the spliceosome, which processes pre-mRNA into mRNA by RNA splicing. Previously, it was found that cisplatin, an FDA-approved chemotherapeutic drug, could inhibit fungal Prp8 intein splicing in vitro and reduce the lung burden of C. neoformans in mice. Li et al., “Cisplatin Protects Mice From Challenge of Cryptococcus neoformans by Targeting the Prp8 Intein,” Emerging Microbes & Infections 8(1):895-908 (2019), which is hereby incorporated by reference in its entirety. The crystal structure of the Cne Prp8 intein in complex with cisplatin (Green et al., “Spliceosomal Prp8 Intein at the Crossroads of Protein and RNA Splicing,” PLoS Biol 17(10):e3000104 (2019) and Li et al., “Cisplatin Protects Mice From Challenge of Cryptococcus neoformans by Targeting the Prp8 Intein,” Emerging Microbes & Infections 8(1):895-908 (2019), both of which are hereby incorporated by reference in their entirety) was also determined, providing the structural basis for cisplatin inhibition of C. neoformans.

In the current study, a split-GFP-based Prp8 intein splicing assay suitable for high throughput screening was developed. Using this assay, a pilot screening of small molecules targeting Prp8 intein splicing was performed and the candidate compounds 6G-318S and 6G-319S were identified as potent inhibitors of Prp8 intein splicing. Both compounds appear specific to Prp8 intein splicing, and do not inhibit representative serine (trypsin) and cysteine (papain) proteases. It was validated that these compounds bind covalently to the Prp8 intein active site residue Cys1, using mass spectrometry, PTSA, mutagenesis experiments, and SPR techniques. Functional studies demonstrated that both compounds are fungicidal and potent inhibitors of the Prp8 intein-containing fungi C. neoformans and C. gattii, but not of C. albicans, which does not encode the Prp8 intein. Their MIC values are comparable to or better than the current frontline drugs for management of infections caused by C. neoformans and C. gattii. These Prp8 intein splicing inhibitors also showed synergistic or additive effects with current frontline antifungals. More importantly, it was shown that treatment of C. neoformans with 6G-318S led to accumulation of unspliced Prp8 protein precursor, supporting the hypothesis that splicing arrest leads to inhibition of the processing of Prp8 precursor protein. Finally, it was demonstrated that overexpression of the Prp8 intein in C. neoformans but not an active-site C1A mutant led to resistance to 6G-318S treatment, reinforcing the contention that the compound acts by targeting the intein active site.

Microbial inteins are attractive drug targets. Li et al., “Cisplatin Protects Mice From Challenge of Cryptococcus neoformans by Targeting the Prp8 Intein,” Emerging Microbes & Infections 8(1):895-908 (2019); Paulus, H. “Protein Splicing Inhibitors as a New Class of Antimycobacterial Agents,” Drugs of the Future 32:973-984 (2007); and Chan et al. “Exploring Intein Inhibition by Platinum Compounds as an Antimicrobial Strategy,” The Journal of Biological Chemistry 291(43):22661-22670 (2016), all of which are hereby incorporated by reference in their entirety. Because processed proteins carry out essential cellular functions in fungi, an intein splicing inhibitor is a mechanistically novel antifungal agent. Intein splicing inhibitors would have the following advantages over traditional antifungal drugs: (1) The Cryptococcus Prp8 inteins share high sequence identity (78-83%). Liu, X. & Yang, J., “Prp8 Intein in Fungal Pathogens: Target for Potential Antifungal Drugs,” FEBS Letters 572(1-3):46-50 (2004), which is hereby incorporated by reference in its entirety. Particularly, the active site residues are absolutely conserved among the Prp8 inteins. Liu, X. & Yang, J., “Prp8 Intein in Fungal Pathogens: Target for Potential Antifungal Drugs,” FEBS Letters 572(1-3):46-50 (2004) and Pearl et al., “Sequence Requirements for Splicing by the Cne PRP8 Intein,” FEBS Letters 581(16):3000-3004 (2007), both of which are hereby incorporated by reference in their entirety. Therefore, it is likely that Prp8 intein splicing inhibitors would be effective against all Cryptococcus fungal species. (2) Humans do not have inteins, suggesting that Prp8 intein inhibitors could be specific to the fungus. This work demonstrates the potential of development of small-molecule inhibitors targeting Prp8 intein splicing and opens a new avenue to develop a novel class of antifungals.

### Example 12—Toxicity Studies of 6G-318S

With increasing report of drug resistance to known antifungal drugs, new drug targets are to be investigated. Cryptococcus neoformans and C. gatti are causative agent in cryptococcosis affecting mainly immunocompromised people. In severe cases it can lead to pulmonary pneumonia and meningitis. Both these species have intein in prp8 protein and intein has to be spliced off for prp8 function which is vital for cell survival. In a protein based intein splicing assay compound 6G-318S (BIONET1_002817) was found to be active in inhibiting the splicing of intein. Also, it inhibits C. neoformans and other related species with minimum inhibitory concentration (MIC) 9₀ of 0.6 to 1 ug/ml.

In vivo toxicity studies in adult BALB/c mice: Solubility and formulation of 6G-318S: The 6G-318S is not water soluble and hence the compound is first dissolved in DMSO. The solution is mixed with corn oil and PEG400 (70% corn oil, 25% PEG, 5% DMSO). Since there was not much knowledge on toxicity of 6G-318S, one mouse each for oral and intraperitoneal IP injection was the starting point.

Mouse 1: Oral, VC—125 ul of formulation without drug is given per mouse by oral gavage

Mouse 2: Oral, 6G-318S—125 ul of formulation 4 mg drug is given by oral gavage (200 mg/kg)

Mouse 3: IP, VC—60 ul of the formulation without the drug is injected intraperitoneally

Mouse 4: IP, 6G-318S—60 ul of the formulation with the compound is injected intraperitoneally (50 mg/kg)

A total of 5 doses were given (one/day). Weight, posture, movement, activity, paralysis, seizure, and fur nature were monitored in the morning and evening.

Result—The mice treated with 6G-318S at 200 mg/kg orally were fine until day 6 on which the mouse was euthanized. The mouse treated with 50 mg/kg intraperitoneally was weak and was euthanized on day 2. To confirm the toxicity, one more mouse was injected intraperitoneal (50 mg/kg) and this mouse also died on day 2, whereas only vehicle treated mice were fine. A third mouse was given a dose of 25 mg/kg intraperitoneally and was fine until day 6 and mouse was euthanized.

Conclusion: 6G-318S at 200 mg/kg oral gavage and 10-20 mg/kg IP dose there is no observed toxicity in BALB/c mice

In vivo preliminary efficacy study—BALB/c mice were infected with C. neoformans (H99) by intranasal route with and without 6G-318S at 75 mg/kg oral gavage once daily with 3-5 mice in each group. Treatment was given for 6 days. On day 6 few mice were euthanized to check the viral load in lungs.

Result—There is an indication of reduction of fungal cells in lungs for the 6G-318S-treated group, compared to the vehicle controls. Overall, the study indicates 6G-318S is safe at 200 mg/kg oral dose and 10-15 mg/kg IP dose.

### Example 13—Synthetic Methods for Anti-Fungal Compound (Prp8 Intein Inhibitors)

General Procedure A:

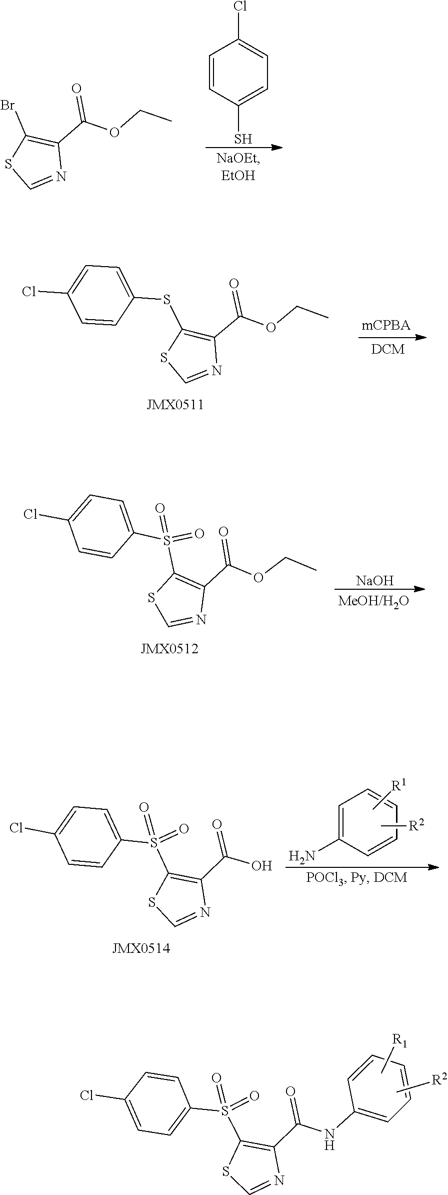

To a solution of ethyl 5-bromothiazole-4-carboxylate (966 mg, 4.09 mmol) and 4-chlorothiopheno (740 mg, 5.17 mmol) in 20 mL of EtOH was added NaOEt (566 mg, 8.18 mmol). The resulting mixture was stirred at 60° C. for 12 h. Then the pH of the mixture was adjusted to 5-6 with 2 M HCl (aq.) and extracted with EtOAc (2×80 mL). The organic phase was washed with brine (40 mL), dried over Na₂SO₄ and concentrated in vacuum. The residue was purified by column chromatography (Hexane/EtOAc=15/1 to 6/1) to afford ethyl 5-((4-chlorophenyl)thio)thiazole-4-carboxylate JMX0511 (1.03 g, 84%) as a light-yellow solid. ¹H NMR (300 MHz, CDC₃) δ 8.47 (s, 1H), 7.63-7.54 (m, 2H), 7.45-7.38 (m, 2H), 4.45 (q, J=7.2 Hz 2H), 1.43 (t, J=7.2 Hz, 3H). ¹³C NMR (75 MHz, CDCl₃) δ 162.3, 149.6, 149.6, 139.3, 137.0, 135.9 (2C), 131.8, 130.5 (2C), 61.8, 14.5.

To a solution of JMX0511 (190 mg, 0.64 mmol) in DCM (10 mL) was added m-chloroperbenzoic acid (409 mg, 1.59 mmol) at 0° C. After addition, the mixture was stirred at r.t overnight. Then the reaction mixture was diluted with DCM (80 mL), washed with Na₂S₂O₃ (aq., 30 mL) and brine (20 mL), dried (Na₂SO₄) and concentrated. The residue was purified by column chromatography (Hexane/EtOAc=3/1) to afford ethyl 5-((4-chlorophenyl)sulfonyl)thiazole-4-carboxylate JMX0512 (190 mg, 90%) as colorless oil. ¹H NMR (300 MHz, CDCl₃) δ 8.99 (s, 1H), 8.05-7.98 (m, 2H), 7.51-7.45 (m, 2H), 4.37 (q, J=7.2 Hz, 2H), 1.34 (t, J=7.2 Hz, 3H). ¹³C NMR (75 MHz, CDCl₃) δ 159.9, 157.2, 147.1, 144.7, 141.0, 138.3, 130.3 (2C), 129.4 (2C), 62.7, 14.0.

To a solution of JMX0512 (190 mg, 0.57 mmol) in MeOH (5 mL) was added NaOH (115 mg, in 3 mL of H₂O). The mixture was stirred at r.t for 1 h and then most of MeOH was evaporated. The pH of the mixture was adjusted to 3-4 with 2 M HCl (aq.) at 0° C. Then the white precipitate was isolated by filtration and dried under vacuum to give 5-((4-chlorophenyl)sulfonyl)thiazole-4-carboxylic acid JMX0514 (140 mg, 80%).

To a solution of acid JMX0514 (1.0 eq), aniline (2.0 eq) and pyridine (20.0 eq) in DCM (5 mL/0.1 mmol) was added POCl₃ (10 eq) at 0° C. The resulting mixture was stirred at r.t overnight. Then the mixture was diluted with DCM, washed with H₂O, and concentrated. The residue was purified by column chromatography (Hex/EtOAc) or crystallization from MeOH to give the corresponding product.

### 5-((4-Chlorophenyl)sulfonyl)-N-(2,4-difluorophenyl)thiazole-4-carboxamide (JMX0515)

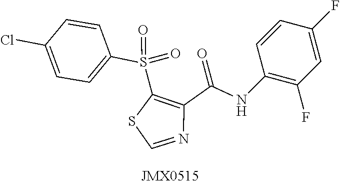 [SMILES: O=C(Nc1ccc(F)cc1F)c1ncsc1S(=O)(=O)c1ccc(Cl)cc1]

20 mg, 21%. White solid. HPLC purity 99.4% (t_{R}=18.46 min). ¹H NMR (300 MHz, DMSO-d₆) δ 10.43 (s, 1H), 9.53 (s, 1H), 8.06 (d, J=8.7 Hz, 2H), 7.77-7.64 (m, 3H), 7.43-7.32 (m, 1H), 7.19-7.08 (m, 1H). HRMS (ESI) calcd for C₁₆H₁₀ClF₂N₂O₃S₂, 414.9789 (M+H)⁺; found, 414.9787.

### 5-((4-Chlorophenyl)sulfonyl)-N-(2-fluorophenyl)thiazole-4-carboxamide (JMX0516)

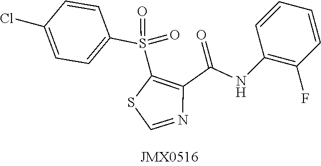 [SMILES: O=C(Nc1ccccc1F)c1ncsc1S(=O)(=O)c1ccc(Cl)cc1]

27 mg, 34%. White solid. HPLC purity 99.2% (t_{R}=18.48 min). ¹H NMR (300 MHz, CDCl₃) δ 9.47 (s, 1H), 8.94 (s, 1H), 8.43 (t, J=7.8 Hz, 1H), 8.23-8.12 (m, 2H), 7.59-7.46 (m, 2H), 7.22-7.05 (m, 3H). ¹³C NMR (75 MHz, CDCl₃) δ 156.1, 155.9, 152.7 (d, J=243.2 Hz), 148.2, 145.2, 141.1, 138.3, 131.1 (2C), 129.3 (2C), 125.8 (d, J=10.0 Hz), 125.2 (d, J=7.6 Hz), 124.8 (d, J=3.7 Hz), 121.7, 115.1 (d, J=18.8 Hz). HRMS (ESI) calcd for C₁₆H₁₁ClFN₂O₃S₂, 396.9884 (M+H)⁺; found, 396.9879.

### 5-((4-Chlorophenyl)sulfonyl)-N-(3-fluorophenyl)thiazole-4-carboxamide (JMX0517)

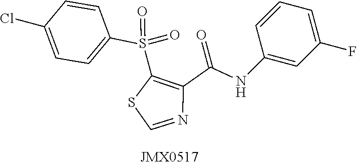 [SMILES: O=C(Nc1cccc(F)c1)c1ncsc1S(=O)(=O)c1ccc(Cl)cc1]

61 mg, 68%. White solid. HPLC purity 99.8% (t_{R}=18.46 min). ¹H NMR (300 MHz, CDCl₃) δ 9.22 (s, 1H), 8.92 (s, 1H), 8.27-8.10 (m, 2H), 7.63 (dt, J=10.5, 2.1 Hz, 1H), 7.57-7.48 (m, 2H), 7.35-7.19 (m, 2H), 6.91-6.78 (m, 1H). ¹³C NMR (75 MHz, CDCl₃) δ 163.1 (d, J=243.8 Hz), 156.2, 155.9, 148.2, 145.3, 141.1, 138.6 (d, J=10.9 Hz), 138.2, 131.1 (2C), 130.3 (d, J=9.3 Hz), 129.3 (2C), 115.3 (d, J=3.0 Hz), 111.9 (d, J=21.2 Hz), 107.5 (d, J=26.3 Hz). HRMS (ESI) calcd for C₁₆H₁₁ClFN₂O₃S₂, 396.9884 (M+H)⁺; found, 396.9877.

### N-(4-Chlorophenyl)-5-((4-chlorophenyl)sulfonyl)thiazole-4-carboxamide (JMX0518)

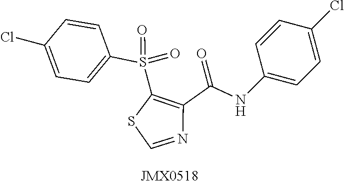 [SMILES: O=C(Nc1ccc(Cl)cc1)c1ncsc1S(=O)(=O)c1ccc(Cl)cc1]

18 mg. Yellow solid. HPLC purity 99.3% (t_{R}=18.99 min). ¹H NMR (300 MHz, CDCl₃) δ 9.18 (s, 1H), 8.92 (s, 1H), 8.21-8.11 (m, 2H), 7.62-7.55 (m, 2H), 7.54-7.48 (m, 2H), 7.35-7.27 (m, 2H). ¹³C NMR (75 MHz, CDCl₃) δ 156.2, 155.9, 148.3, 145.1, 141.1, 138.3, 135.7, 131.1 (2C), 130.2, 129.3 (4C), 121.3 (2C). HRMS (ESI) calcd for C₁₆H₁₁Cl₂N₂O₃S₂, 412.9588 (M+H)⁺; found, 412.9588.

### 5-((4-Chlorophenyl)sulfonyl)-N-(2,4-dichlorophenyl)thiazole-4-carboxamide (JMX0519)

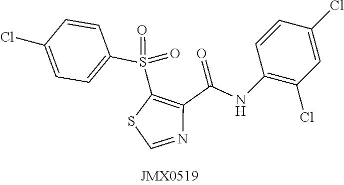 [SMILES: O=C(Nc1ccc(Cl)cc1Cl)c1ncsc1S(=O)(=O)c1ccc(Cl)cc1]

81 mg, 79%. White Solid. HPLC purity 99.3% (t_{R}=20.72 min). ¹H NMR (300 MHz, DMSO-d₆) δ 10.28 (s, 1H), 9.55 (s, 1H), 8.14-8.02 (m, 2H), 7.88 (d, J=8.7 Hz, 1H), 7.76-7.67 (m, 3H), 7.50 (dd, J=8.7, 2.4 Hz, 1H). ¹³C NMR (75 MHz, DMSO-d₆) δ 160.4, 157.7, 149.0, 141.7, 139.4, 138.7, 133.0, 130.2, 130.1, 129.4, 129.1, 127.9, 127.3, 126.0. HRMS (ESI) calcd for C₁₆H₁₀Cl₃N₂O₃S₂, 446.9198 (M+H)⁺; found, 446.9195.

### 5-((4-Chlorophenyl)sulfonyl)-N-(3,4-difluorophenyl)thiazole-4-carboxamide (JMX0520)

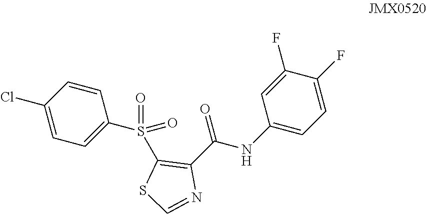 [SMILES: O=C(Nc1ccc(F)c(F)c1)c1ncsc1S(=O)(=O)c1ccc(Cl)cc1]

72 mg, 75%. Grey solid. HPLC purity 99.5% (t_{R}=18.65 min). ¹H NMR (300 MHz, DMSO-d₆) δ 10.91 (s, 1H), 9.54 (s, 1H), 8.16-8.01 (m, 2H), 7.89-7.66 (m, 3H), 7.57-7.36 (m, 2H). ¹³C NMR (75 MHz, DMSO-d₆) δ 160.6, 158.6, 150.6, 148.9 (dd, J=242.2, 13.2 Hz), 146.0 (dd, J=241.2, 12.6 Hz), 140.2, 139.5, 138.8, 135.0 (dd, J=8.9, 3.0 Hz), 130.1 (2C), 129.6 (2C), 117.6 (d, J=17.7 Hz), 116.7 (dd, J=6.0, 3.5 Hz), 109.2 (d, J=21.4 Hz). HRMS (ESI) calcd for C₁₆H₁₀ClF₂N₂O₃S₂, 414.9789 (M+H)⁺; found, 414.9785.

### 5-((4-Chlorophenyl)sulfonyl)-N-(4-(trifluoromethyl)phenyl)thiazole-4-carboxamide (JMX0521)

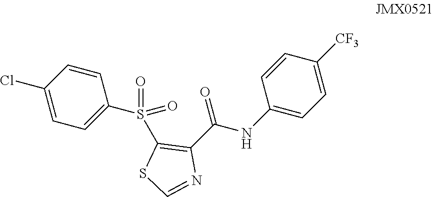 [SMILES: O=C(Nc1ccc(C(F)(F)F)cc1)c1ncsc1S(=O)(=O)c1ccc(Cl)cc1]

51 mg, 50%. White Solid. HPLC purity 98.6% (t_{R}=19.40 min). ¹H NMR (300 MHz, CDCl₃) δ 9.32 (s, 1H), 8.94 (s, 1H), 8.24-8.13 (m, 2H), 7.76 (d, J=8.7 Hz, 2H), 7.61 (d, J=8.7 Hz, 2H), 7.56-7.49 (m, 2H). ¹³C NMR (75 MHz, CDCl₃) δ 156.4, 155.9, 148.0, 145.6, 141.2, 140.2, 138.2, 131.1 (2C), 129.3 (2C), 126.9 (d, J=32.7 Hz), 126.5 (d, J=3.7 Hz, 2C), 124.1 (d, J=270.0 Hz), 119.8 (2C). HRMS (ESI) calcd for C17H₁₁ClF₃N₂O₃S₂, 446.9852 (M+H)⁺; found, 446.9850.

### 5-((4-Chlorophenyl)sulfonyl)-N-(3-fluoro-5-(trifluoromethyl)phenyl) thiazole-4-carboxamide (JMX0522)

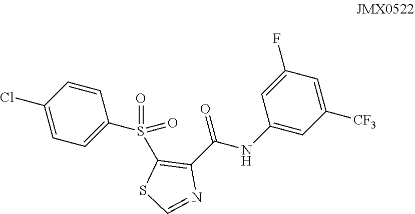 [SMILES: O=C(Nc1cc(F)cc(C(F)(F)F)c1)c1ncsc1S(=O)(=O)c1ccc(Cl)cc1]

62 mg, 58%. Off-white solid. HPLC purity 98.9% (t_{R}=19.78 min). ¹H NMR (300 MHz, CDCl₃) δ 9.37 (s, 1H), 8.94 (s, 1H), 8.27-8.11 (m, 2H), 7.87 (dt, J=10.2, 2.1 Hz, 1H), 7.58-7.54 (m, 1H), 7.54-7.50 (m, 2H), 7.11 (d, J=8.1 Hz, 1H). ¹³C NMR (75 MHz, CDCl₃) δ 162.9 (d, J=246.5 Hz), 156.4, 156.0, 147.5, 145.9, 141.4, 139.3 (d, J=11.0 Hz), 138.1, 133.0 (qd, J=33.5, 9.2 Hz), 131.1 (2C), 129.4 (2C), 123.2 (qd, J=270.8, 3.2 Hz), 112.3 (qui, J=3.6 Hz), 110.6 (d, J=26.3 Hz), 109.1 (dq, J=24.7, 3.8 Hz). HRMS (ESI) calcd for C₁₇H₁₀ClF₄N₂O₃S₂, 464.9757 (M+H)⁺; found, 464.9761.

### 5-((4-Chlorophenyl)sulfonyl)-N-(6-(trifluoromethyl)pyridin-3-yl) thiazole-4-carboxamide (JMX0523)

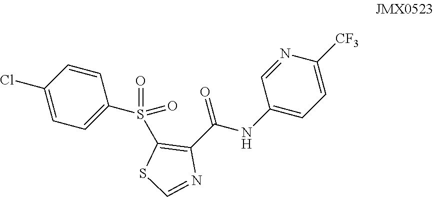 [SMILES: O=C(Nc1ccc(C(F)(F)F)nc1)c1ncsc1S(=O)(=O)c1ccc(Cl)cc1]

57 mg, 55%. White solid. HPLC purity 98.5% (t_{R}=18.27 min). ¹H NMR (300 MHz, CDCl₃) δ 9.41 (s, 1H), 8.96 (s, 1H), 8.73 (d, J=2.4 Hz, 1H), 8.51 (dd, J=8.7, 2.4 Hz, 1H), 8.22-8.11 (m, 2H), 7.70 (d, J=8.4 Hz, 1H), 7.58-7.47 (m, 2H). ¹³C NMR (75 MHz, CDCl₃) δ 156.8, 156.1, 147.1, 146.3, 144.1 (q, J=35.1 Hz), 141.4, 141.1, 138.0, 136.3, 131.1 (2C), 129.4 (2C), 127.4, 121.6 (q, J=271.9 Hz), 121.2 (q, J=2.6 Hz). HRMS (ESI) calcd for C₁₆H₁₀ClF₃N₃O₃S₂, 447.9804 (M+H)⁺; found, 447.9803.

### 5-((4-Chlorophenyl)sulfonyl)-N-(6-fluoropyridin-3-yl)thiazole-4-carboxamide (JMX0524)

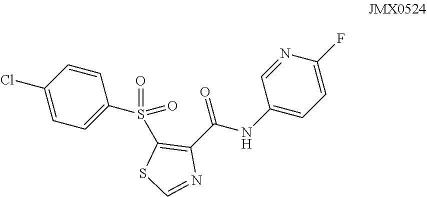 [SMILES: O=C(Nc1ccc(F)nc1)c1ncsc1S(=O)(=O)c1ccc(Cl)cc1]

52 mg, 57%. Light-yellow solid. HPLC purity 96.0% (t_{R}=17.03 min). ¹H NMR (300 MHz, CDCl₃+CD₃OD) δ 9.16 (s, 1H), 8.44 (d, J=1.8 Hz, 1H), 8.30-8.21 (m, 1H), 8.16-8.07 (m, 2H), 7.56-7.51 (m, 2H), 6.99 (dd, J=8.7, 3.0 Hz, 1H). ¹³C NMR (75 MHz, CDCl₃+CD₃OD) δ 160.7 (d, J=237.0 Hz), 158.5, 158.3, 149.3, 144.4, 141.5, 139.7 (d, J=14.3 Hz), 139.0, 134.7 (d, J=7.9 Hz), 133.2 (d, J=4.6 Hz), 131.2 (2C), 129.8 (2C), 110.1 (d, J=37.9 Hz). HRMS (ESI) calcd for C₁₅H₁₀ClFN₃O₃S₂, 397.9836 (M+H)⁺; found, 397.9832.

### 5-((4-Chlorophenyl)sulfonyl)-N-(4-fluorophenyl)thiazole-4-carboxamide (JMX0525)

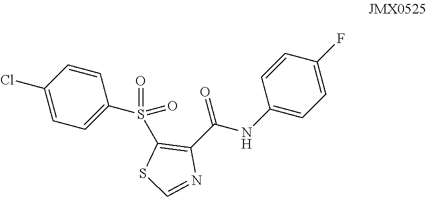 [SMILES: O=C(Nc1ccc(F)cc1)c1ncsc1S(=O)(=O)c1ccc(Cl)cc1]

73 mg, 80%. Grey solid. HPLC purity 99.4% (t_{R}=18.12 min). ¹H NMR (300 MHz, CDCl₃) δ 9.14 (s, 1H), 8.92 (s, 1H), 8.24-8.09 (m, 2H), 7.64-7.55 (m, 2H), 7.55-7.48 (m, 2H), 7.09-6.99 (m, 2H). ¹³C NMR (75 MHz, CDCl₃) δ 159.9 (d, J=242.0 Hz), 156.2, 155.8, 148.5, 145.0, 141.1, 138.4, 133.1 (d, J=2.9 Hz), 131.1 (2C), 129.3 (2C), 121.8 (d, J=8.0 Hz, 2C), 116.0 (d, J=22.4 Hz, 2C). HRMS (ESI) calcd for C₁₆H₁₁ClFN₂O₃S₂, 396.9884 (M+H)⁺; found, 396.9877.

### 5-((4-Chlorophenyl)sulfonyl)-N-(4-fluorobenzyl)thiazole-4-carboxamide (JMX0541)

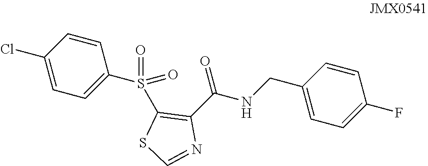 [SMILES: O=C(NCc1ccc(F)cc1)c1ncsc1S(=O)(=O)c1ccc(Cl)cc1]

66 mg, 95%. White solid. HPLC purity 99.2% (t_{R}=17.64 min). ¹H NMR (300 MHz, CDCl₃) δ 8.84 (s, 1H), 8.20-8.06 (m, 2H), 7.63-7.44 (m, 3H), 7.27-7.18 (m, 2H), 7.04
6.93 (m, 2H), 4.51 (d, J=6.0 Hz, 2H). ¹³C NMR (75 MHz, CDCl₃) δ 162.4 (d, J=244.7 Hz), 158.5, 156.1, 148.7, 144.0, 140.9, 138.5, 133.3 (d, J=3.2 Hz), 130.9 (2C), 129.7 (d, J=8.1 Hz, 2C), 129.2 (2C), 115.7 (d, J=21.4 Hz, 2C), 43.0. HRMS (ESI) calcd for C₁₇H₁₃ClFN₂O₃S₂, 411.0040 (M+H)⁺; found, 411.0036.

General Procedure B:

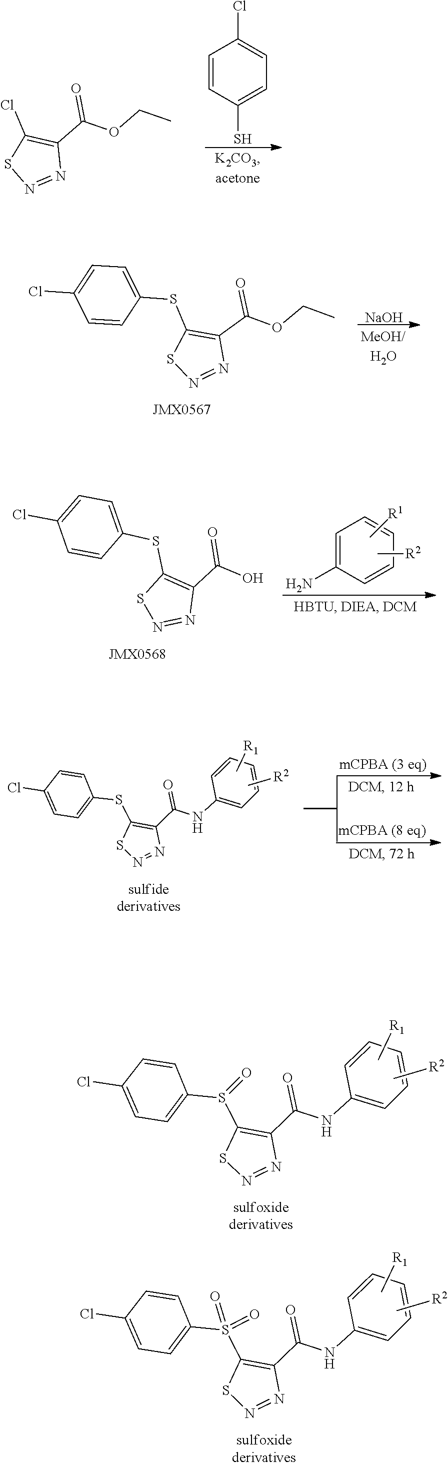

To a solution of ethyl 5-chloro-1,2,3-thiadiazole-4-carboxylate (270 mg, 1.40 mmol) and 4-chlorothiopheno (405 mg, 2.80 mmol) in 20 mL of acetone was added K₂CO₃ (387 mg, 2.80 mmol) at 0° C. The resulting mixture was stirred at 0° C. for 2 h. Then the mixture was diluted with EtOAc (100 mL), washed with H₂O (20 mL) and brine (20 mL), dried over Na₂SO₄ and concentrated in vacuum. The residue was purified by column chromatography (Hexane/EtOAc=20/1 to 5/1) to afford ethyl 5-((4-chlorophenyl)thio)-1,2,3-thiadiazole-4-carboxylate JMX0567 (390 mg, 92%) as a white solid. ¹H NMR (300 MHz, CDCl₃) δ 7.53 (d, J=8.4 Hz, 2H), 7.38 (d, J=8.4 Hz, 2H), 4.41 (q, J=7.2 Hz, 2H), 1.36 (t, J=7.2 Hz, 3H). ¹³C NMR (75 MHz, CDCl₃) δ 168.3, 160.4, 145.2, 137.7, 135.2 (2C), 130.8 (2C), 130.7, 62.0, 14.1.

To a solution of ethyl 5-((4-chlorophenyl)thio)-1,2,3-thiadiazole-4-carboxylate JMX0567 (390 mg, 1.30 mmol) in MeOH (5 mL) was added NaOH (104 mg, 2.60 mmol, in 3 mL of H₂O). The mixture was stirred at r.t for 2 h and then most of MeOH was evaporated. The pH of the mixture was adjusted to 3-4 with 2 M HCl (aq.) at 0° C. The mixture was extracted with EtOAc (2×80 mL), washed with H₂O (30 mL) and brine (30 mL), dried over Na₂SO₄ and concentrated in vacuum to give the product 5-((4-chlorophenyl)thio)-1,2,3-thiadiazole-4-carboxylic acid JMX0568 (340 mg, 96%) as a white solid.

To a solution of 5-((4-chlorophenyl)thio)-1,2,3-thiadiazole-4-carboxylic acid JMX0568 (1.0 eq), aniline (1.2 eq) and DIEA (2.0 eq) in DCM (5 mL/0.1 mmol) was added HBTU (1.5 eq). The resulting mixture was stirred at r.t for 24 h and then concentrated. The residue was purified by column chromatography to give the sulfide derivatives.

The sulfide intermediate (1 eq) was dissolved in DCM (5 mL/0.1 mmol), and mCPBA (3 eq) was added. The resultant mixture was stirred at r.t for 12 h. Then the reaction mixture was diluted with DCM (80 mL/0.1 mmol), washed with Na₂S₂O₃ (aq., 30 mL/0.1 mmol) and brine (20 mL/0.1 mmol), dried (Na₂SO₄) and concentrated. The residue was purified by column chromatography (Hex/EtOAc) or crystallization from MeOH to give the sulfoxide derivatives.

The sulfide intermediate (1 eq) was dissolved in DCM (5 mL/0.1 mmol), and mCPBA (10 eq) was added. The resultant mixture was stirred at r.t for 72 h. Then the reaction mixture was diluted with DCM (80 mL/0.1 mmol), washed with Na₂S₂O₃ (aq., 30 mL/0.1 mmol) and brine (20 mL/0.1 mmol), dried (Na₂SO₄) and concentrated. The residue was purified by column chromatography (Hex/EtOAc) or crystallization from MeOH to give the sulphone derivatives.

### 5-((4-Chlorophenyl)thio)-N-(4-fluorophenyl)-1,2,3-thiadiazole-4-carboxamide (JMX0564)

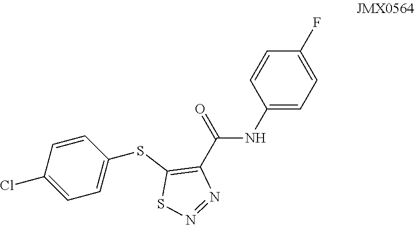 [SMILES: O=C(Nc1ccc(F)cc1)c1nnsc1Sc1ccc(Cl)cc1]

The 70 mg, 66%. White solid. HPLC purity 99.2% (t_{R}=19.92 min). ¹H NMR (300 MHz, CDCl₃) δ 9.16 (s, 1H), 7.76-7.60 (m, 4H), 7.50 (d, J=8.4 Hz, 2H), 7.09 (t, J=8.7 Hz, 2H). ¹³C NMR (75 MHz, CDCl₃) δ 166.7, 159.9 (d, J=243.4 Hz), 158.3, 147.6, 138.2, 135.5 (2C), 133.4 (d, J=2.8 Hz), 131.6, 131.2 (2C), 121.8 (d, J=8.0 Hz, 2C), 116.0 (d, J=22.5 Hz, 2C). HRMS (ESI) calcd for C₁₅H₁₀ClFN₃OS₂, 365.9938 (M+H)⁺; found, 365.9935.

### 5-((4-Chlorophenyl)sulfinyl)-N-(4-fluorophenyl)-1,2,3-thiadiazole-4-carboxamide (JMX0565-1)

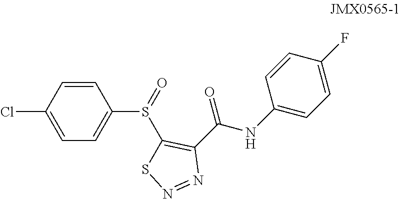 [SMILES: O=C(Nc1ccc(F)cc1)c1nnsc1S(=O)c1ccc(Cl)cc1]

40 mg, 64%. Yellow solid. HPLC purity 99.7% (t_{R}=18.35 min). ¹H NMR (300 MHz, CDCl₃) δ 9.24 (s, 1H), 8.01 (d, J=8.4 Hz, 2H), 7.71-7.62 (m, 2H), 7.49 (d, J=8.4 Hz, 2H), 7.12 (t, J=8.4 Hz, 2H). ¹³C NMR (75 MHz, CDCl₃) δ 174.9, 160.2 (d, J=244.2 Hz), 156.2, 151.4, 142.4, 139.1, 132.7 (d, J=2.9 Hz), 130.1 (2C), 127.2 (2C), 122.2 (d, J=8.0 Hz, 2C), 116.2 (d, J=22.7 Hz, 2C). HRMS (ESI) calcd for C₁₅H₁₀ClFN₃O₂S₂, 381.9887 (M+H)⁺; found, 381.9885.

### 5-((4-Chlorophenyl)sulfonyl)-N-(4-fluorophenyl)-1,2,3-thiadiazole-4-carboxamide (JMX0565-2)

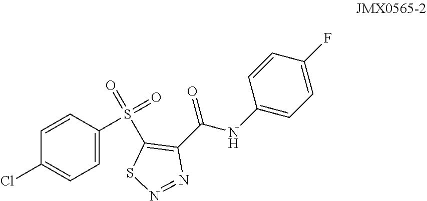 [SMILES: O=C(Nc1ccc(F)cc1)c1nnsc1S(=O)(=O)c1ccc(Cl)cc1]

50 mg, 76%. White solid. HPLC purity 99.6% (t_{R}=18.50 min). ¹H NMR (300 MHz, CDCl₃) δ 9.23 (s, 1H), 8.26-8.10 (m, 2H), 7.66-7.58 (m, 2H), 7.58-7.52 (m, 2H), 7.11-7.02 (m, 2H). ¹³C NMR (75 MHz, CDCl₃) δ 162.8, 160.1 (d, J=244.0 Hz), 154.6, 152.1, 142.3, 136.9, 132.7 (d, J=2.9 Hz), 131.5 (2C), 129.7 (2C), 122.2 (d, J=8.0 Hz, 2C), 116.2 (d, J=22.6 Hz, 2C). HRMS (ESI) calcd for C₁₅H₁₀ClFN₃O₃S₂, 397.9836 (M+H)⁺; found, 397.9836.

### 5-((4-Chlorophenyl)thio)-N-(2,4-difluorophenyl)-1,2,3-thiadiazole-4-carboxamide (JMX0569)

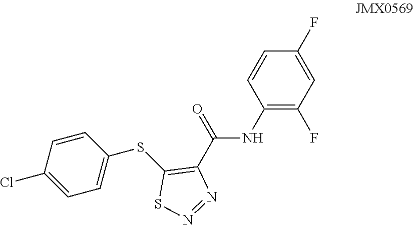 [SMILES: O=C(Nc1ccc(F)cc1F)c1nnsc1Sc1ccc(Cl)cc1]

80 mg, 99%. White solid. HPLC purity 95.7% (t_{R}=20.12 min). ¹H NMR (300 MHz, CDCl₃) δ 9.31 (s, 1H), 8.50-8.39 (m, 1H), 7.66 (d, J=8.4 Hz, 2H), 7.51 (d, J=8.4 Hz, 2H), 6.95 (t, J=8.4 Hz, 2H). ¹³C NMR (75 MHz, CDCl₃) δ 167.0, 159.3 (dd, J=245.9, 11.3 Hz), 158.3, 153.0 (dd, J=246.4, 11.8 Hz), 147.4, 138.3, 135.5 (2C), 131.5, 131.3 (2C), 122.9 (dd, J=8.9, 1.9 Hz), 122.3 (dd, J=10.6, 3.8 Hz), 111.6 (dd, J=21.6, 3.8 Hz), 104.1 (dd, J=26.5, 23.0 Hz). HRMS (ESI) calcd for C₁₅H₉ClF₂N₃OS₂, 383.9844 (M+H)⁺; found, 383.9843.

### N-(4-Chlorophenyl)-5-((4-chlorophenyl)thio)-1,2,3-thiadiazole-4-carboxamide (JMX0570)

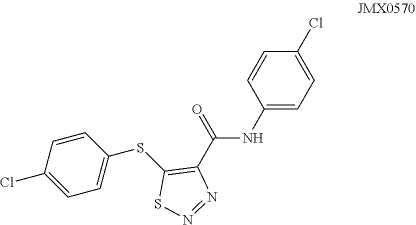 [SMILES: O=C(Nc1ccc(Cl)cc1)c1nnsc1Sc1ccc(Cl)cc1]

65 mg, 92%. White solid. HPLC purity 99.1% (t_{R}=20.74 min). ¹H NMR (300 MHz, CDCl₃) δ 9.19 (s, 1H), 7.76-7.58 (m, 4H), 7.50 (d, J=7.2 Hz, 2H), 7.35 (d, J=7.5 Hz, 2H). ¹³C NMR (75 MHz, CDCl₃) δ 166.9, 158.3, 147.5, 138.2, 135.9, 135.5 (2C), 131.5, 131.2 (2C), 130.1, 129.4 (2C), 121.2 (2C). HRMS (ESI) calcd for C₁₅H₁₀Cl₂N₃OS₂, 381.9642 (M+H)⁺; found, 381.9633.

### N-(4-Chlorophenyl)-5-((4-chlorophenyl)sulfonyl)-1,2,3-thiadiazole-4-carboxamide (JMX0571)

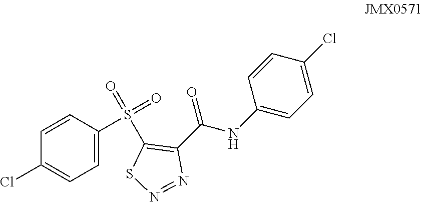 [SMILES: O=C(Nc1ccc(Cl)cc1)c1nnsc1S(=O)(=O)c1ccc(Cl)cc1]

29 mg, 58%. Yellow solid. HPLC purity 99.3% (t_{R}=19.27 min). ¹H NMR (300 MHz, CDCl₃) δ 9.24 (s, 1H), 8.21 (d, J=8.7 Hz, 2H), 7.64-7.52 (m, 4H), 7.34 (d, J=8.7 Hz, 2H). ¹³C NMR (75 MHz, CDCl₃) δ 163.0, 154.5, 151.9, 142.4, 136.9, 135.3, 131.5 (2C), 130.8, 129.7 (2C), 129.4 (2C), 121.6 (2C). HRMS (ESI) calcd for C₁₅H₁₀Cl₂N₃O₃S₂, 413.9541 (M+H)⁺; found, 413.9539.

### 5-((4-Chlorophenyl)sulfonyl)-N-(2,4-difluorophenyl)-1,2,3-thiadiazole-4-carboxamide (JMX0572)

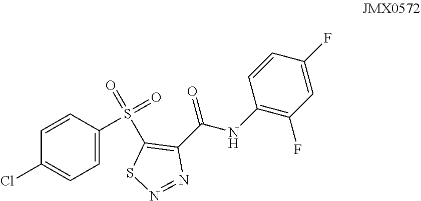 [SMILES: O=C(Nc1ccc(F)cc1F)c1nnsc1S(=O)(=O)c1ccc(Cl)cc1]

57 mg, 79%. White solid. HPLC purity 99.8% (t_{R}=18.52 min). ¹H NMR (300 MHz, CDCl₃) δ 9.38 (s, 1H), 8.44-8.08 (m, 3H), 7.56 (d, J=7.8 Hz, 2H), 7.02-6.82 (m, 2H). ¹³C NMR (75 MHz, CDCl₃) δ 163.1, 159.6 (dd, J=246.7, 11.3 Hz), 154.6, 153.0 (dd, J=247.0, 11.7 Hz), 151.6, 142.4, 136.9, 131.5 (2C), 129.7 (2C), 123.2 (dd, J=9.1, 1.3 Hz), 121.6 (dd, J=10.5, 3.8 Hz), 111.6 (dd, J=21.9, 3.7 Hz), 104.1 (dd, J=26.6, 22.9 Hz). HRMS (ESI) calcd for C₁₅H₉ClF₂N₃O₃S₂, 415.9742 (M+H)⁺; found, 415.9739.

### 5-((4-Chlorophenyl)thio)-N-(4-fluorobenzyl)-1,2,3-thiadiazole-4-carboxamide (JMX0573)

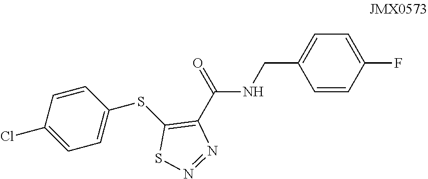 [SMILES: O=C(NCc1ccc(F)cc1)c1nnsc1Sc1ccc(Cl)cc1]

62 mg, 88%. White solid. HPLC purity 97.9% (t_{R}=19.31 min). ¹H NMR (300 MHz, CDCl₃) δ 7.76 (s, 1H), 7.61 (d, J=8.4 Hz, 2H), 7.46 (d, J=8.4 Hz, 2H), 7.39-7.30 (m, 2H), 7.01 (t, J=8.7 Hz, 2H), 4.65 (d, J=6.0 Hz, 2H). ¹³C NMR (75 MHz, CDCl₃) δ 165.8, 162.4 (d, J=244.5 Hz), 160.2, 147.5, 137.9, 135.4 (2C), 133.5 (d, J=3.1 Hz), 131.7, 131.1 (2C), 129.7 (d, J=8.1 Hz, 2C), 115.7 (d, J=21.4 Hz, 2C), 42.8. HRMS (ESI) calcd for C₁₆H₁₂ClFN₃OS₂, 380.0094 (M+H)⁺; found, 380.0092.

### 5-((4-Chlorophenyl)sulfonyl)-N-(4-fluorobenzyl)-1,2,3-thiadiazole-4-carboxamide (JMX0574)

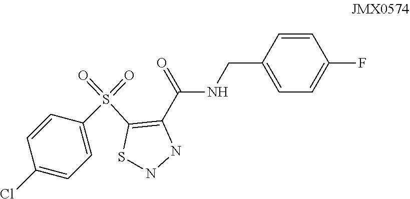 [SMILES: O=C(NCc1ccc(F)cc1)C1=C(S(=O)(=O)c2ccc(Cl)cc2)SNN1]

45 mg, 83%. White solid. HPLC purity 99.5% (t_{R}=18.08 min). ¹H NMR (300 MHz, CDCl₃) δ 8.19 (d, J=8.7 Hz, 2H), 7.65 (s, 1H), 7.56 (d, J=8.7 Hz, 2H), 7.32-7.26 (m, 2H), 7.03 (t, J=8.7 Hz, 2H), 4.61 (d, J=6.0 Hz, 2H). ¹³C NMR (75 MHz, CDCl₃) δ 162.5 (d, J=245.0 Hz), 162.1, 156.8, 152.1, 142.2, 137.1, 132.8 (d, J=3.2 Hz), 131.4 (2C), 129.8 (d, J=8.1 Hz), 129.6 (2C), 115.9 (d, J=21.5 Hz), 43.3. HRMS (ESI) calcd for C₁₆H12ClFN₃O₃S₂, 411.9993 (M+H)⁺; found, 411.9991.

### 5-((4-Chlorophenyl)thio)-N-(4-(trifluoromethyl)phenyl)-1,2,3-thiadiazole-4-carboxamide (JMX0575)

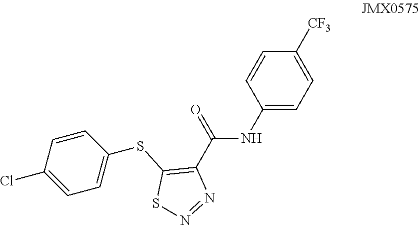 [SMILES: O=C(Nc1ccc(C(F)(F)F)cc1)c1nnsc1Sc1ccc(Cl)cc1]

86 mg, 95%. Light-yellow solid. HPLC purity 97.2% (t_{R}=21.10 min). ¹H NMR (300 MHz, CDCl₃) δ 9.33 (s, 1H), 7.86 (d, J=8.4 Hz, 2H), 7.64 (d, J=8.4 Hz, 4H), 7.50 (d, J=8.4 Hz, 2H). ¹³C NMR (75 MHz, CDCl₃) δ 167.4, 158.4, 147.30 140.4, 138.3, 135.4 (2C), 131.4, 131.3 (2C), 126.8 (q, J=33.0 Hz), 126.6 (q, J=3.6 Hz, 2C), 124.1 (q, J=270.0 Hz), 119.6 (2C). HRMS (ESI) calcd for C₁₆H₁₀ClF₃N₃OS₂, 415.9906 (M+H)⁺; found, 415.9909.

### 5-((4-Chlorophenyl)sulfonyl)-N-(4-(trifluoromethyl)phenyl)-1,2,3-thiadiazole-4-carboxamide (JMX0576)

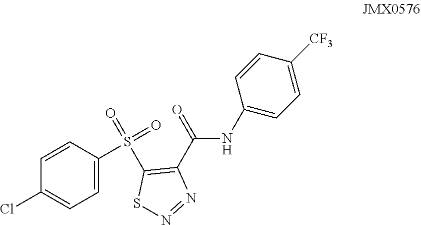 [SMILES: O=C(Nc1ccc(C(F)(F)F)cc1)c1nnsc1S(=O)(=O)c1ccc(Cl)cc1]

64 mg, 85%. White solid. HPLC purity 99.6% (t_{R}=19.60 min). ¹H NMR (300 MHz, CDCl₃) δ 9.40 (s, 1H), 8.27-8.17 (m, 2H), 7.79 (d, J=8.4 Hz, 2H), 7.63 (d, J=8.7 Hz, 2H), 7.60-7.53 (m, 2H). ¹³C NMR (75 MHz, CDCl₃) δ 163.4, 154.8, 151.7, 142.5, 139.7, 136.8, 131.5 (2C), 129.7 (2C), 127.4 (q, J=32.6 Hz), 126.6 (q, J=3.8 Hz, 2C), 124.0 (q, J=270.1 Hz), 120.1 (2C). HRMS (ESI) calcd for C₁₆H₁₀ClF₃N₃O₃S₂, 447.9804 (M+H)⁺; found, 447.9806.

### 5-((4-Chlorophenyl)thio)-N-(6-fluoropyridin-3-yl)-1,2,3-thiadiazole-4-carboxamide (JMX0580)

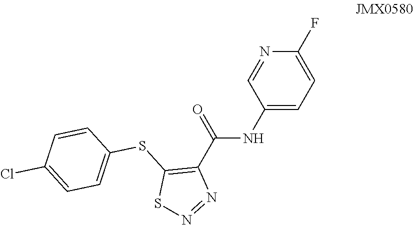 [SMILES: O=C(Nc1ccc(F)nc1)c1nnsc1Sc1ccc(Cl)cc1]

65 mg, 88%. Yellow solid. HPLC purity 98.3% (t_{R}=18.75 min). ¹H NMR (300 MHz, CDCl₃) δ 9.28 (s, 1H), 8.45 (s, 1H), 8.41-8.32 (m, 1H), 7.64 (d, J=8.4 Hz, 2H), 7.50 (d, J=8.4 Hz, 2H), 6.98 (dd, J=8.7, 3.0 Hz, 1H). ¹³C NMR (75 MHz, CDCl₃) δ 167.4, 160.3 (d, J=236.6 Hz), 158.5, 147.0, 139.0, 138.8, 138.3, 135.4 (2C), 133.1 (d, J=7.7 Hz), 132.1 (d, J=4.7 Hz), 131.3 (2C), 109.8 (d, J=38.7 Hz). HRMS (ESI) calcd for C₁₄H₉ClFN₄OS₂, 366.9890 (M+H)⁺; found, 366.9885.

### 5-((4-Chlorophenyl)sulfonyl)-N-(6-fluoropyridin-3-yl)-1,2,3-thiadiazole-4-carboxamide (JMX0581)

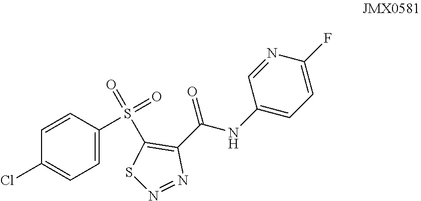 [SMILES: O=C(Nc1ccc(F)nc1)c1nnsc1S(=O)(=O)c1ccc(Cl)cc1]

55 mg, 84%. White solid. HPLC purity 98.0% (t_{R}=17.23 min). ¹H NMR (300 MHz, CDCl₃) δ 9.29 (s, 1H), 8.40 (s, 1H), 8.34-8.27 (m, 1H), 8.21 (d, J=8.7 Hz, 2H), 7.58 (d, J=8.7 Hz, 2H), 7.00 (dd, J=9.0, 3.3 Hz, 1H). ¹³C NMR (75 MHz, CDCl₃) δ 163.3, 160.6 (d, J=237.5 Hz), 155.0, 151.4, 142.5, 139.4 (d, J=15.4 Hz), 136.7, 133.5 (d, J=7.8 Hz), 131.5 (d, J=5.0 Hz), 131.5 (2C), 129.7 (2C), 109.9 (d, J=38.7 Hz). HRMS (ESI) calcd for C₁₄H₉ClFN₄O₃S₂, 398.9789 (M+H)⁺; found, 398.9782.

### Synthesis of 5-((4-chlorophenyl)sulfonyl)-N-(4-fluorophenyl)-1,3,4-thiadiazole-2-carboxamide (JMX0585-1) and 5-((4-chlorophenyl)sulfinyl)-N-(4-fluorophenyl)-1,3,4-thiadiazole-2-carboxamide (JMX0585-2)

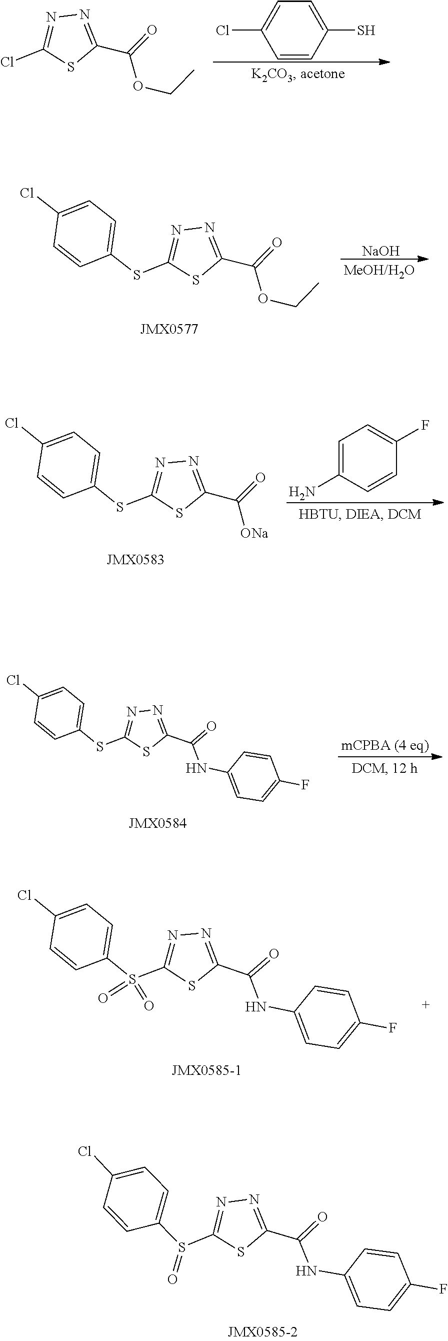

To a solution of ethyl 5-chloro-1,3,4-thiadiazole-2-carboxylate (350 mg, 1.82 mmol) and 4-chlorothiopheno (525 mg, 3.63 mmol) in 20 mL of acetone was added K₂CO₃ (502 mg, 3.63 mmol) at 0° C. The resulting mixture was stirred at 0° C. for 3 h. Then the mixture was diluted with EtOAc (100 mL), washed with H₂O (20 mL) and brine (20 mL), dried over Na₂SO₄ and concentrated in vacuum. The residue was purified by column chromatography (Hexane/EtOAc=20/1 to 5/1) to afford ethyl 5-((4-chlorophenyl)thio)-1,3,4-thiadiazole-2-carboxylate JMX0577 (560 mg, 99%) as a white solid. ¹H NMR (300 MHz, CDCl₃) δ 7.65-7.56 (m, 2H), 7.48-7.38 (m, 2H), 4.41 (q, J=7.2 Hz, 2H), 1.36 (t, J=7.2 Hz, 3H). ¹³C NMR (75 MHz, CDCl₃) δ 174.6, 160.0, 158.1, 137.7, 136.0 (2C), 130.7 (2C), 128.0, 63.3, 14.1.

To a solution of ethyl 5-((4-chlorophenyl)thio)-1,3,4-thiadiazole-2-carboxylate JMX0577 (210 mg, 0.70 mmol) in MeOH (6 mL) was added NaOH (56 mg, 1.40 mmol, in 4 mL of H₂O). The mixture was stirred at r.t for 2 h and then most of MeOH was evaporated. The white precipitate was isolated by filtration. 200 mg (97%) of sodium 5-((4-chlorophenyl)thio)-1,3,4-thiadiazole-2-carboxylate (JMX0583) was afforded as a white solid. ¹H NMR (300 MHz, CD₃OD) δ 7.71 (d, J=8.5 Hz, 2H), 7.53 (d, J=8.5 Hz, 2H). ¹³C NMR (75 MHz, CD₃OD) S 172.9, 171.1, 163.1, 138.1, 137.0 (2C), 131.5 (2C), 130.4.

To a solution of sodium 5-((4-chlorophenyl)thio)-1,3,4-thiadiazole-2-carboxylate (JMX0583) (100 mg, 0.34 mmol) and 4-fluoroaniline (75 mg, 0.68 mmol) in 15 mL of DCM was added HBTU (217 mg, 0.68 mmol) and DIPEA (88 mg, 0.68 mmol) at 0° C. The resulting mixture was stirred at r.t overnight. And then concentrated. The residue was purified by column chromatography (Hexane/EtOAc=4/1) to give 5-((4-chlorophenyl)thio)-N-(4-fluorophenyl)-1,3,4-thiadiazole-2-carboxamide (JMX0584) (84 mg, 68%) as a yellow solid. HPLC purity 99.3% (t_{R}=19.36 min). ¹H NMR (300 MHz, CDCl₃) δ 8.94 (s, 1H), 7.71-7.55 (m, 4H), 7.47 (d, J=8.7 Hz, 2H), 7.06 (t, J=8.7 Hz, 2H). ¹³C NMR (75 MHz, CDCl₃) δ 175.1, 165.3, 160.1 (d, J=243.8 Hz), 155.1, 137.9, 136.1 (2C), 132.7 (d, J=2.9 Hz), 130.9 (2C), 128.1, 121.9 (d, J=8.0 Hz, 2C), 116.2 (d, J=22.6 Hz, 2C). HRMS (ESI) calcd for C₁₅H₁₀ClFN₃OS₂, 365.9938 (M+H)⁺; found, 365.9933.

To a solution of 5-((4-chlorophenyl)thio)-N-(4-fluorophenyl)-1,3,4-thiadiazole-2-carboxamide (JMX0584) (70 mg, 0.19 mmol) in DCM (15 mL) was added m-chloroperbenzoic acid (197 mg, 0.77 mmol) at 0° C. After addition, the mixture was stirred at r.t overnight. Then the reaction mixture was diluted with DCM (80 mL), washed with Na₂S₂O₃ (aq., 30 mL) and brine (20 mL), dried (Na₂SO₄) and concentrated. The residue was purified by column chromatography (Hexane/EtOAc=3/1) to afford 5-((4-chlorophenyl)sulfonyl)-N-(4-fluorophenyl)-1,3,4-thiadiazole-2-carboxamide (JMX0585-1) (55 mg, 72%) as a yellow solid. HPLC purity 99.3% (t_{R}=18.51 min). ¹H NMR (300 MHz, CDCl₃) δ 9.00 (s, 1H), 8.09 (d, J=8.7 Hz, 2H), 7.72-7.49 (m, 4H), 7.09 (t, J=8.7 Hz, 2H). ¹³C NMR (75 MHz, CDCl₃) δ 174.1, 169.9, 160.4 (d, J=244.7 Hz), 154.1, 142.9, 136.1, 132.2 (d, J=2.9 Hz), 130.8 (2C), 130.4 (2C), 122.2 (d, J=8.1 Hz, 2C), 116.4 (d, J=22.7 Hz, 2C). HRMS (ESI) calcd for C₁₅H₁₀ClFN₃O₃S2 397.9836 (M+H)⁺, found 397.9835. And 5-((4-chlorophenyl)sulfinyl)-N-(4-fluorophenyl)-1,3,4-thiadiazole-2-carboxamide (JMX0585-2) (5 mg, 7%) as a white solid. HPLC purity 95.2% (t_{R}=17.78 min). ¹H NMR (300 MHz, CDCl₃) δ 8.96 (s, 1H), 7.82 (d, J=8.7 Hz, 2H), 7.66-7.53 (m, 4H), 7.09 (t, J=8.7 Hz, 2H). ¹³C NMR (75 MHz, CDCl₃) δ 185.0, 168.8, 160.3 (d, J=244.4 Hz), 154.5, 140.8, 139.4, 132.3 (d, J=2.9 Hz), 130.5 (2C), 125.8 (2C), 122.1 (d, J=8.0 Hz, 2C), 116.3 (d, J=22.7 Hz, 2C). HRMS (ESI) calcd for C₁₅H₁₀ClFN₃O₂S₂, 381.9887 (M+H)⁺; found, 381.9882.

### 5-((4-Chlorophenyl)sulfonyl)-N-(3-fluorophenyl)-1,2,3-thiadiazole-4-carboxamide (JMX0871)

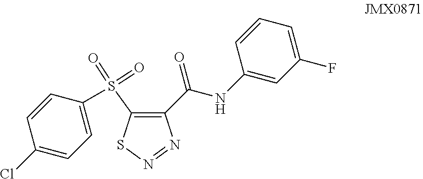 [SMILES: O=C(Nc1cccc(F)c1)c1nnsc1S(=O)(=O)c1ccc(Cl)cc1]

60 mg, 68%. Yellow solid. ¹H NMR (300 MHz, CDCl₃) δ 9.30 (s, 1H), 8.28-8.16 (m, 2H), 7.65 (dt, J=10.5, 2.1 Hz, 1H), 7.60-7.53 (m, 2H), 7.37-7.24 (m, 2H), 6.94-6.85 (m, 1H). ¹³C NMR (75 MHz, CDCl₃) δ 163.1 (d, J=244.1 Hz), 163.1, 154.6, 151.8, 142.4, 138.1 (d, J=10.8 Hz), 136.8, 131.5 (2C), 130.5 (d, J=9.3 Hz), 129.7 (2C), 115.6 (d, J=3.0 Hz), 112.4 (d, J=21.2 Hz), 107.9 (d, J=26.5 Hz).

### 5-((4-Chlorophenyl)sulfonyl)-N-(2-fluorophenyl)-1,2,3-thiadiazole-4-carboxamide (JMX0872)

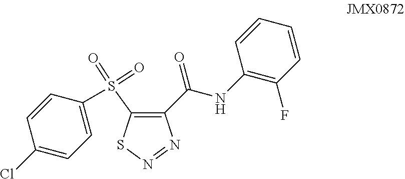 [SMILES: O=C(Nc1ccccc1F)c1nnsc1S(=O)(=O)c1ccc(Cl)cc1]

38 mg, 70%. Yellow solid. ¹H NMR (300 MHz, CDCl₃) δ 9.50 (s, 1H), 8.45-8.35 (m, 1H), 8.27-8.17 (m, 2H), 7.61-7.50 (m, 2H), 7.24-7.10 (m, 3H). ¹³C NMR (75 MHz, CDCl₃) δ 163.0, 154.5, 152.8 (d, J=244.1 Hz), 151.8, 142.3, 136.9, 131.5 (2C), 129.7 (2C), 125.9 (d, J=7.7 Hz), 125.3 (d, J=10.2 Hz), 124.8 (d, J=3.8 Hz), 122.0, 115.3 (d, J=18.8 Hz).

### 5-((4-Chlorophenyl)sulfonyl)-N-(3,4-difluorophenyl)-1,2,3-thiadiazole-4-carboxamide (JMX0876)

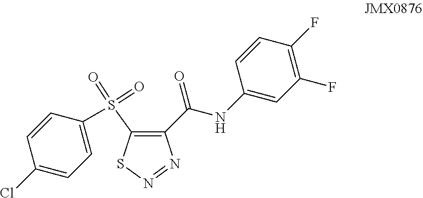 [SMILES: O=C(Nc1ccc(F)c(F)c1)c1nnsc1S(=O)(=O)c1ccc(Cl)cc1]

64 mg, 68%. yellow solid. ¹H NMR (300 MHz, CDCl₃) δ 9.25 (s, 1H), 8.26-8.15 (m, 2H), 7.84-7.74 (m, 1H), 7.62-7.53 (m, 2H), 7.25-7.11 (m, 2H). ¹³C NMR (75 MHz, CDCl₃) δ 163.2, 154.6, 151.6, 150.3 (dd, J=246.7, 13.2 Hz), 147.9 (dd, J=245.9, 12.7 Hz), 142.5, 136.8, 133.1 (dd, J=8.8, 3.4 Hz), 131.5 (2C), 129.7 (2C), 117.1 (dd, J=18.4, 1.1 Hz), 116.0 (dd, J=6.1, 3.7 Hz), 110.2 (d, J=21.9 Hz).

### Synthesis of 5-((4-chlorophenyl)sulfonyl)-N-(p-tolyl)-1,2,3-thiadiazole-4-carboxamide (JMX1033)

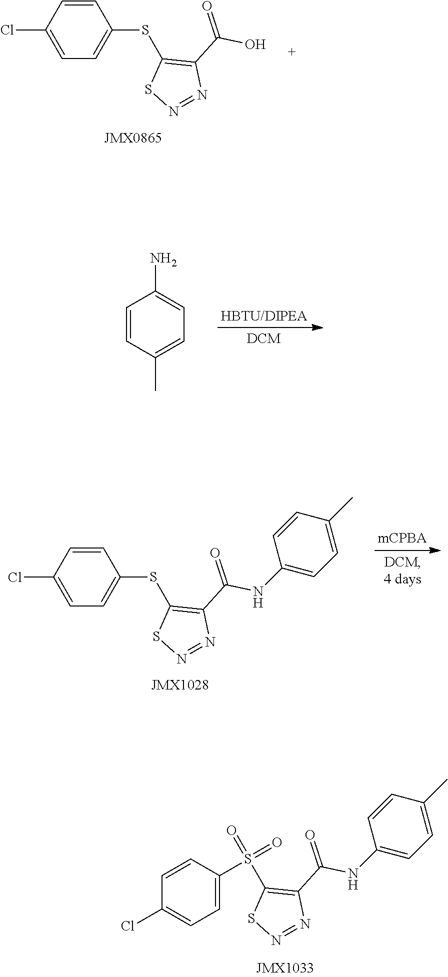

To a solution of JMX0865 (50 mg, 0.16 mmol) and p-toluidine (35 mg, 0.33 mmol) in 5 mL of DMF was added DIEA (42 mg, 0.33 mmol) and HBTU (125 mg, 0.33 mmol) at 0° C. The resulting mixture was stirred at r.t overnight and diluted with 80 mL DCM. The mixture was washed with H₂O (2×30 mL) and concentrated. Then 2 mL of MeOH and 6 mL of H₂O were added. The mixture was stirred at r.t. for 10 min and the white solid was isolated by filtration to give JMX1028 which was used for the next step without further purification. The white solid was dissolved in 15 mL of DCM, and m-chloroperbenzoic acid (202 mg, 0.82 mmol) was added at 0° C. The resulting mixture was stirred at r.t. for 4 days. Then the reaction mixture was diluted with DCM (60 mL), washed with Na₂S₂O₃ (aq., 20 mL) and brine (20 mL), dried (Na₂SO₄) and concentrated. The residue was purified by preparative TLC (Hexane/DCM=1/1) to afford 5-((4-chlorophenyl)sulfonyl)-N-(p-tolyl)-1,2,3-thiadiazole-4-carboxamide (JMX1033) (31 mg, 48% in two steps) as a pale yellow solid. ¹H NMR (300 MHz, CDCl₃) δ 9.19 (s, 1H), 8.26-8.20 (m, 2H), 8.03 (d, J=8.1 Hz, 1H), 7.59-7.52 (m, 2H), 7.31-7.21 (m, 2H), 7.18-7.11 (m, 1H), 2.33 (s, 3H). ¹³C NMR (75 MHz, CDCl₃) δ 162.7, 154.6, 152.5, 142.2, 137.0, 134.7, 131.6 (2C), 130.8, 129.6 (2C), 129.0, 127.1, 126.1, 122.5, 17.8.

### N-(4-Bromophenyl)-5-((4-chlorophenyl)sulfonyl)-1,2,3-thiadiazole-4-carboxamide (JMX1034)

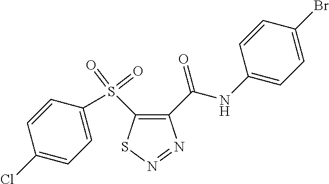 [SMILES: O=C(Nc1ccc(Br)cc1)c1nnsc1S(=O)(=O)c1ccc(Cl)cc1]

44 mg, 58% in two steps. Pale yellow solid. ¹H NMR (300 MHz, CDCl₃) δ 9.24 (s, 1H), 8.21 (d, J=8.4 Hz, 2H), 7.61-7.46 (m, 6H). ¹³C NMR (75 MHz, CDCl₃) δ 163.0, 154.5, 151.9, 142.4, 136.9, 135.8, 132.4 (2C), 131.5 (2C), 129.7 (2C), 121.8 (2C), 118.5.

### 5-((4-Chlorophenyl)sulfonyl)-N-phenyl-1,2,3-thiadiazole-4-carboxamide (JMX1035)

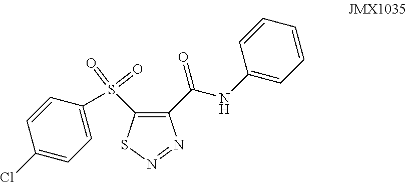 [SMILES: O=C(Nc1ccccc1)c1nnsc1S(=O)(=O)c1ccc(Cl)cc1]

47 mg, 75% in two steps. Yellow solid. ¹H NMR (300 MHz, CDCl₃) δ 9.25 (s, 1H), 8.22 (d, J=8.4 Hz, 2H), 7.65 (d, J=8.4 Hz, 2H), 7.55 (d, J=8.4 Hz, 2H), 7.43-7.34 (m, 2H), 7.20 (t, J=7.2 Hz, 1H). ¹³C NMR (75 MHz, CDCl₃) δ 162.7, 154.5, 152.2, 142.2, 136.9, 136.7, 131.5 (2C), 129.6 (2C), 129.3 (2C), 125.7, 120.3 (2C).

### N-(3-Chloro-5-fluorophenyl)-5-((4-chlorophenyl)sulfonyl)-1,2,3-thiadiazole-4-carboxamide (JMX1036)

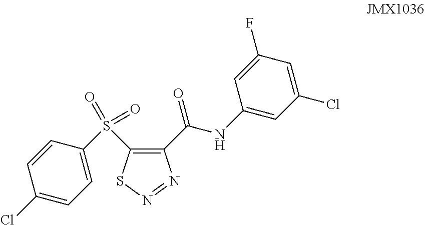 [SMILES: O=C(Nc1cc(F)cc(Cl)c1)c1nnsc1S(=O)(=O)c1ccc(Cl)cc1]

45 mg, 63% in two steps. White solid. ¹H NMR (300 MHz, CDCl₃) δ 9.30 (s, 1H), 8.21 (d, J=8.7 Hz, 2H), 7.59 (d, J=8.7 Hz, 2H), 7.52-7.42 (m, 2H), 6.93 (dt, J=8.1, 2.1 Hz, 1H). ¹³C NMR (75 MHz, CDCl₃) δ 163.6, 162.9 (d, J=247.3 Hz), 154.7, 151.4, 142.5, 138.7 (d, J=12.2 Hz), 136.7, 135.7 (d, J=12.2 Hz), 131.6 (2C), 129.8 (2C), 116.0 (d, J=3.3 Hz), 113.3 (d, J=24.8 Hz), 106.2 (d, J=26.4 Hz).

### N-(3-Chloro-4-fluorophenyl)-5-((4-chlorophenyl)sulfonyl)-1,2,3-thiadiazole-4-carboxamide (JMX1037)

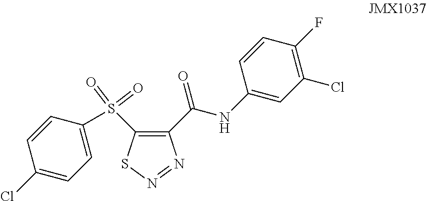 [SMILES: O=C(Nc1ccc(F)c(Cl)c1)c1nnsc1S(=O)(=O)c1ccc(Cl)cc1]

51 mg, 72% in two steps. Yellow solid. ¹H NMR (300 MHz, CDCl₃) δ 9.23 (s, 1H), 8.22 (d, J=8.7 Hz, 2H), 7.90 (dd, J=6.3, 2.7 Hz, 1H), 7.58 (d, J=8.7 Hz, 2H), 7.48-7.41 (m, 1H), 7.15 (t, J=8.7 Hz, 1H). ¹³C NMR (75 MHz, CDCl₃) δ 163.2, 155.6 (d, J=246.6 Hz), 154.6, 151.6, 142.5, 136.8, 133.3 (d, J=3.5 Hz), 131.5 (2C), 129.8 (2C), 122.6, 121.8 (d, J=18.5 Hz), 120.0 (d, J=6.9 Hz), 117.1 (d, J=22.2 Hz).

### 5-((4-Chlorophenyl)sulfonyl)-N-(3,5-difluorophenyl)-1,2,3-thiadiazole-4-carboxamide (JMX1038)

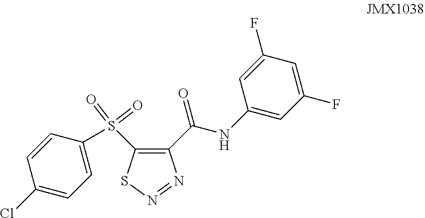 [SMILES: O=C(Nc1cc(F)cc(F)c1)c1nnsc1S(=O)(=O)c1ccc(Cl)cc1]

44 mg, 64% in two steps. Yellow solid. ¹H NMR (300 MHz, CDCl₃) δ 9.30 (s, 1H), 8.25-8.18 (m, 2H), 7.62-7.56 (m, 2H), 7.32-7.27 (m, 2H), 6.66 (tt, J=8.7, 2.4 Hz, 1H). ¹³C NMR (75 MHz, CDCl₃) δ 163.6, 163.4 (dd, J=246.1, 4.3 Hz), 154.6 (d, J=6.2 Hz), 151.4 (d, J=2.0 Hz), 142.5, 138.9-138.5 (m), 136.7, 131.6 (2C), 129.8 (2C), 103.7-103.2 (m, 3C), 101.0 (t, J=25.4 Hz).

### Synthesis of 5-((4-chlorophenyl)sulfonyl)-N-(3-fluoro-5-(trifluoromethyl)phenyl)-1,2,3-thiadiazole-4-carboxamide (JMX1040)

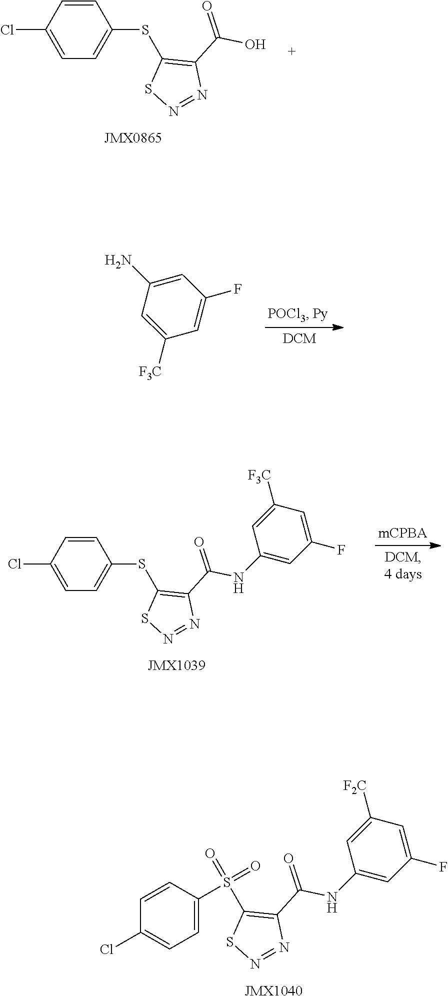

To a solution of JMX0865 (60 mg, 0.20 mmol), 3-fluoro-5-(trifluoromethyl)aniline (71 mg, 0.39 mmol) and pyridine (312 mg, 1.97 mmol) in 15 mL of DCM was added POCl₃ (302 mg, 1.97 mmol) at 0° C. The resulting mixture was stirred at r.t. for 12 h. Then diluted with DCM (60 mL), washed with NAHCO₃ (aq., 20 mL), and concentrated. MeOH (2 mL) and H₂O (6 mL) were added and the mixture was stirred at r.t. for 10 min. The white solid was isolated to afford the intermediate JMX1039 which was used for the next step without further purification. The intermediate was dissolved in 20 mL of DCM, and m-chloroperbenzoic acid (243 mg, 0.99 mmol) was added at 0° C. The resulting mixture was stirred at r.t. for 4 days. Then the reaction mixture was diluted with DCM (60 mL), washed with Na₂S₂O₃ (aq., 20 mL) and brine (20 mL), dried (Na₂SO₄) and concentrated. The residue was purified by preparative TLC (Hexane/DCM=1/2) to afford 5-((4-chlorophenyl)sulfonyl)-N-(3-fluoro-5-(trifluoromethyl)phenyl)-1,2,3-thiadiazole-4-carboxamide (JMX1040) (67 mg, 73% in two steps) as a white solid. ¹H NMR (300 MHz, CDCl₃) δ 9.49 (s, 1H), 8.26-8.16 (m, 2H), 7.86 (dt, J=10.2, 1.8 Hz, 1H), 7.63-7.53 (m, 3H), 7.15 (d, J=8.1 Hz, 1H). ¹³C NMR (75 MHz, CDCl₃) δ 163.6, 162.8 (d, J=246.9 Hz), 154.9, 151.2, 142.6, 138.9 (d, J=11.0 Hz), 136.6, 133.1 (qd, J=33.5, 9.3 Hz), 131.5 (2C), 129.8 (2C), 123.0 (qd, J=271.0, 3.3 Hz), 112.6 (quint, J=3.6 Hz), 110.9 (d, J=26.4 Hz), 109.6 (dq, J=24.6, 3.6 Hz).

### N-(3,5-Bis(trifluoromethyl)phenyl)-5-((4-chlorophenyl)sulfonyl)-1,2,3-thiadiazole-4-carboxamide (JMX1041)

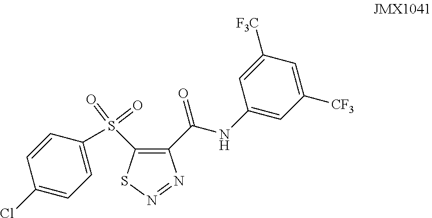 [SMILES: O=C(Nc1cc(C(F)(F)F)cc(C(F)(F)F)c1)c1nnsc1S(=O)(=O)c1ccc(Cl)cc1]

70 mg, 69% in two steps. Pale yellow solid. ¹H NMR (300 MHz, CDCl₃) δ 9.53 (s, 1H), 8.27-8.20 (m, 2H), 8.18 (s, 2H), 7.71 (s, 1H), 7.63-7.56 (m, 2H). ¹³C NMR (75 MHz, CDCl₃) δ 163.9, 155.0, 151.0, 142.7, 138.2, 136.7, 132.9 (q, J=33.5 Hz, 2C), 131.5 (2C), 129.9 (2C), 123.1 (q, J=271.2 Hz, 2C), 120.0 (q, J=3.3 Hz, 2C), 119.0 (hept, J=3.8 Hz).

### N-(2-Chloro-4-(trifluoromethyl)phenyl)-5-((4-chlorophenyl)sulfonyl)-1,2,3-thiadiazole-4-carboxamide (JMX1042)

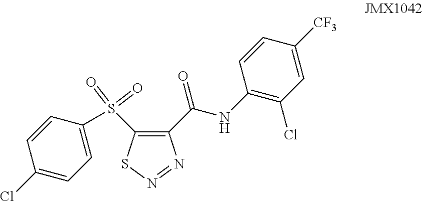 [SMILES: O=C(Nc1ccc(C(F)(F)F)cc1Cl)c1nnsc1S(=O)(=O)c1ccc(Cl)cc1]

70 mg, 73% in two steps. Pale yellow solid. ¹H NMR (300 MHz, CDCl₃) δ 10.05 (s, 1H), 8.67 (d, J=8.4 Hz, 1H), 8.28-8.16 (m, 2H), 7.70 (d, J=1.5 Hz, 1H), 7.63-7.54 (m, 3H).

### 5-((4-Chlorophenyl)sulfonyl)-N-(2-fluoro-4-nitrophenyl)-1,2,3-thiadiazole-4-carboxamide (JMX1043)

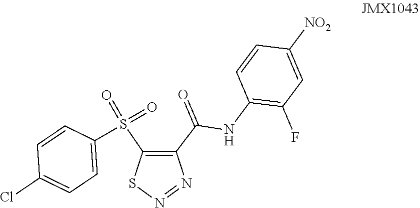 [SMILES: O=C(Nc1ccc([N+](=O)[O-])cc1F)c1nnsc1S(=O)(=O)c1ccc(Cl)cc1]

53 mg, 73% in two steps. Yellow solid. ¹H NMR (300 MHz, CDCl₃) δ 9.77 (d, J=2.1 Hz, 1H), 8.72 (dd, J=9.0, 7.8 Hz, 1H), 8.26-8.18 (m, 2H), 8.18-8.11 (m, 1H), 8.07 (dd, J=10.5, 2.4 Hz, 1H), 7.64-7.55 (m, 2H). ¹³C NMR (75 MHz, CDCl₃) δ 164.2, 154.8, 151.3 (d, J=248.0 Hz), 150.8, 144.0 (d, J=8.4 Hz), 142.7, 136.6, 131.5 (d, J=9.9 Hz), 131.5 (2C), 129.8 (2C), 120.9, 120.9 (d, J=3.1 Hz), 111.4 (d, J=24.1 Hz).

### 5-((4-Chlorophenyl)sulfonyl)-N-(3,4,5-trifluorophenyl)-1,2,3-thiadiazole-4-carboxamide (JMX1044)

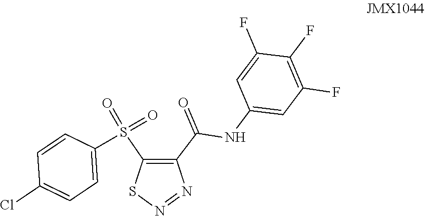 [SMILES: O=C(Nc1cc(F)c(F)c(F)c1)c1nnsc1S(=O)(=O)c1ccc(Cl)cc1]

45 mg, 63% in two steps. Yellow solid. ¹H NMR (300 MHz, CDCl₃) δ 9.24 (s, 1H), 8.26-8.17 (m, 2H), 7.64-7.55 (m, 2H), 7.48-7.34 (m, 2H). ¹³C NMR (75 MHz, CDCl₃+CD₃OD) δ 161.8, 155.5, 152.3, 150.9 (ddd, J=246.3, 10.1, 5.3 Hz), 142.2, 138.8-138.2 (m, 0.5C), 136.7, 135.5-135.0 (m, 0.5C), 133.1-132.6 (m), 131.1 (2C), 129.6 (2C), 105.2-104.7 (m, 3C).

### 5-((4-Chlorophenyl)sulfinyl)-N-(3,4,5-trifluorophenyl)-1,2,3-thiadiazole-4-carboxamide (JMX1044-2)

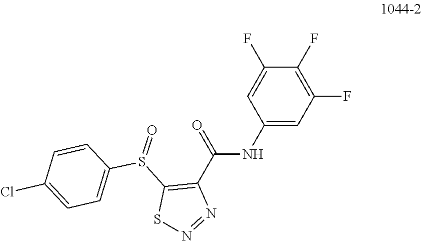 [SMILES: O=C(Nc1cc(F)c(F)c(F)c1)c1nnsc1S(=O)c1ccc(Cl)cc1]

2 mg, 3% in two steps. Yellow solid. ¹H NMR (300 MHz, CDCl₃) δ 9.16 (s, 1H), 8.01-7.95 (m, 2H), 7.53-7.48 (m, 2H), 7.46-7.39 (m, 2H).

### 5-((4-Chlorophenyl)sulfonyl)-N-(2,4,6-trifluorophenyl)-1,2,3-thiadiazole-4-carboxamide (JMX1045)

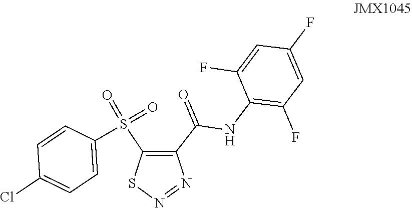 [SMILES: O=C(Nc1c(F)cc(F)cc1F)c1nnsc1S(=O)(=O)c1ccc(Cl)cc1]

38 mg, 53% in two steps. Yellow solid. ¹H NMR (300 MHz, CDCl₃) δ 8.69 (s, 1H), 8.22-8.13 (m, 2H), 7.60-7.50 (m, 2H), 6.87-6.74 (m, 2H).

### 5-((4-Chlorophenyl)sulfinyl)-N-(2,4,6-trifluorophenyl)-1,2,3-thiadiazole-4-carboxamide (JMX1045-2)

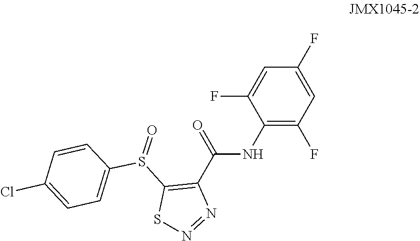 [SMILES: O=C(Nc1c(F)cc(F)cc1F)c1nnsc1S(=O)c1ccc(Cl)cc1]

7 mg, 10% in two steps. Pale-yellow solid. ¹H NMR (300 MHz, CDCl₃) δ 8.64 (s, 1H), 8.00-7.93 (m, 2H), 7.53-7.43 (m, 2H), 6.90-6.78 (m, 2H).

### N-(4-Chloro-3-fluorophenyl)-5-((4-chlorophenyl)sulfonyl)-1,2,3-thiadiazole-4-carboxamide (JMX1046)

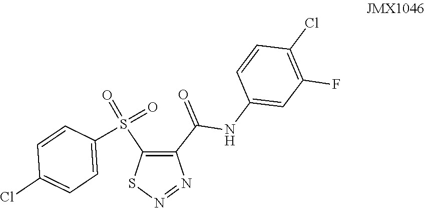 [SMILES: O=C(Nc1ccc(Cl)c(F)c1)c1nnsc1S(=O)(=O)c1ccc(Cl)cc1]

43 mg, 60% in two steps. Pale yellow solid. ¹H NMR (300 MHz, CDCl₃) δ 9.30 (s, 1H), 8.26-8.16 (m, 2H), 7.79 (dd, J=10.8, 2.4 Hz, 1H), 7.61-7.55 (m, 2H), 7.42-7.34 (m, 1H), 7.25-7.21 (m, 1H). ¹³C NMR (75 MHz, CDCl₃) δ 163.4, 158.3 (d, J=246.8 Hz), 154.6, 151.5, 142.5, 136.8, 136.5 (d, J=9.7 Hz), 131.6 (2C), 131.0, 129.8 (2C), 117.5 (d, J=17.8 Hz), 116.3 (d, J=3.6 Hz), 109.0 (d, J=26.1 Hz).

### N-(4-Chloro-3-fluorophenyl)-5-((4-chlorophenyl)sulfinyl)-1,2,3-thiadiazole-4-carboxamide (JMX1046-2)

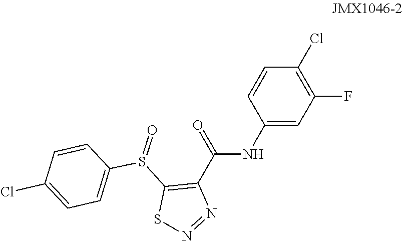 [SMILES: O=C(Nc1ccc(Cl)c(F)c1)c1nnsc1S(=O)c1ccc(Cl)cc1]

12 mg, 17% in two steps. Pale yellow solid. ¹H NMR (300 MHz, CDCl₃) δ 9.23 (s, 1H), 8.03-7.95 (m, 2H), 7.81 (dd, J=10.5, 2.4 Hz, 1H), 7.53-7.47 (m, 2H), 7.41 (dd, J=8.7, 7.9 Hz, 1H), 7.45-7.38 (m, 1H), 7.29-7.23 (m, 1H). ¹³C NMR (75 MHz, CDCl₃) δ 175.5, 158.3 (d, J=247.1 Hz), 156.2, 150.8, 142.2, 139.3, 136.5 (d, J=9.8 Hz), 131.1, 130.2, 130.1, 127.3, 127.2, 117.6 (d, J=18.0 Hz), 116.0 (d, J=2.8 Hz), 109.0 (dd, J=26.4, 6.8 Hz).

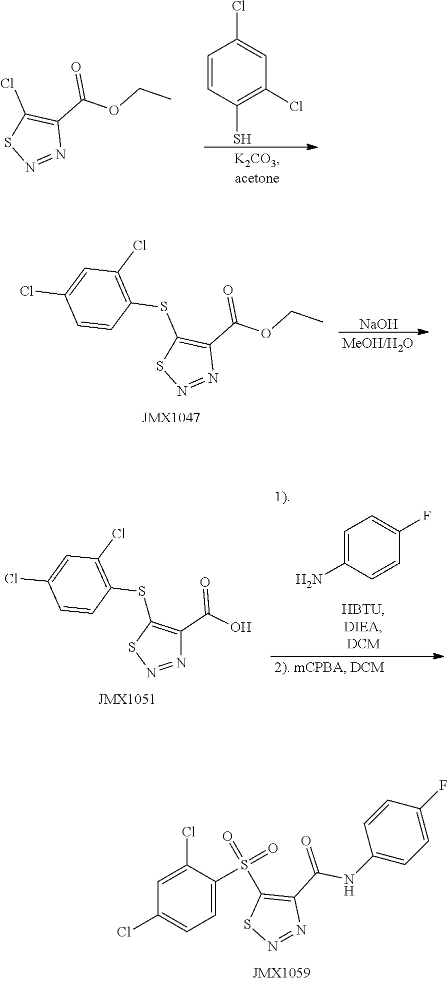

### Ethyl 5-((2,4-dichlorophenyl)thio)-1,2,3-thiadiazole-4-carboxylate (JMX1047)

500 mg, 95%. White solid. ¹H NMR (300 MHz, CDCl₃) δ 7.72 (d, J=8.4 Hz, 1H), 7.65 (d, J=2.1 Hz, 1H), 7.40 (dd, J=8.4, 2.4 Hz, 1H), 4.56 (q, J=7.2 Hz, 2H), 1.50 (t, J=7.2 Hz, 3H).

### 5-((2,4-Dichlorophenyl)thio)-1,2,3-thiadiazole-4-carboxylic acid (JMX1051)

450 mg, 94% in two steps. White solid. ¹H NMR (300 MHz, CDCl₃) δ 7.74 (d, J=8.4 Hz, 1H), 7.66 (d, J=2.1 Hz, 1H), 7.41 (dd, J=8.4, 2.1 Hz, 1H).

### 5-((2,4-Dichlorophenyl)sulfonyl)-N-(4-fluorophenyl)-1,2,3-thiadiazole-4-carboxamide (JMX1059)

75 mg, 88% in two steps. Yellow solid. ¹H NMR (300 MHz, CDCl₃) δ 9.23 (s, 1H), 8.51 (d, J=8.7 Hz, 1H), 7.62-7.50 (m, 3H), 7.46 (d, J=2.0 Hz, 1H), 7.10-6.98 (m, 2H). ¹³C NMR (75 MHz, CDCl₃) δ 161.6, 160.1 (d, J=244.0 Hz), 154.4, 152.3, 142.4, 135.2, 134.9, 133.5, 132.6 (d, J=2.9 Hz), 131.5, 127.7, 122.1 (d, J=8.0 Hz, 2C), 116.1 (d, J=22.6 Hz, 2C).

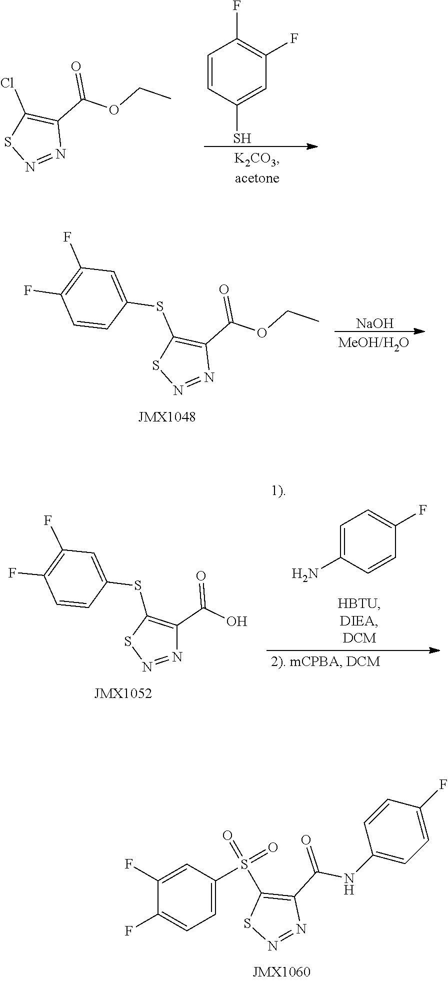

### Ethyl 5-((3,4-difluorophenyl)thio)-1,2,3-thiadiazole-4-carboxylate (JMX1048)

White solid. ¹H NMR (300 MHz, CDCl₃) δ 7.61-7.44 (m, 2H), 7.39-7.28 (m, 1H), 4.55 (q, J=7.2 Hz, 2H), 1.50 (t, J=7.2 Hz, 3H).

### 5-((3,4-Difluorophenyl)thio)-1,2,3-thiadiazole-4-carboxylic acid (JMX1052)

440 mg, 95% in two steps. White solid. ¹H NMR (300 MHz, CDCl₃) δ 7.61-7.45 (m, 2H), 7.42-7.30 (m, 1H).

### 5-((3,4-Difluorophenyl)sulfonyl)-N-(4-fluorophenyl)-1,2,3-thiadiazole-4-carboxamide (JMX1060)

62 mg, 71% in two steps. Grey solid. ¹H NMR (300 MHz, CDCl₃) δ 9.24 (s, 1H), 8.21-8.13 (m, 1H), 8.12-8.05 (m, 1H), 7.68-7.59 (m, 2H), 7.44-7.32 (m, 1H), 7.13-7.03 (m, 2H). ¹³C NMR (75 MHz, CDCl₃) δ 162.3, 160.2 (d, J=244.1 Hz), 154.9 (dd, J=260.0, 12.5 Hz), 154.5, 150.1 (dd, J=254.3, 13.3 Hz), 152.1, 135.1 (dd, J=5.1, 3.7 Hz), 132.6 (d, J=2.9 Hz), 127.7 (dd, J=8.1, 3.9 Hz), 122.2 (d, J=8.0 Hz, 2C), 120.2 (dd, J=20.4, 2.2 Hz), 118.5 (d, J=18.6 Hz), 116.1 (d, J=22.6 Hz, 2C).

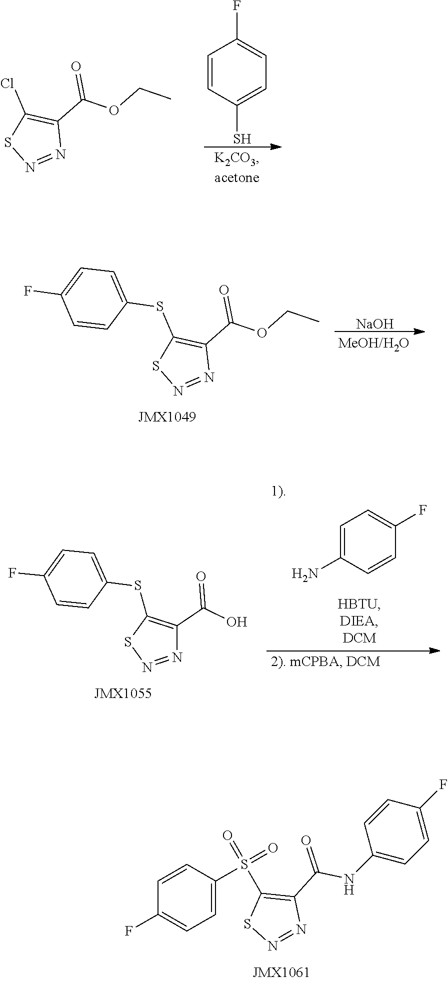

### Ethyl 5-((4-fluorophenyl)thio)-1,2,3-thiadiazole-4-carboxylate (JMX1049)

White solid. ¹H NMR (300 MHz, CDCl₃) δ 7.73-7.65 (m, 2H), 7.25-7.16 (m, 2H), 4.55 (q, J=7.2 Hz, 2H), 1.49 (t, J=7.2, 3H).

### 5-((4-Fluorophenyl)thio)-1,2,3-thiadiazole-4-carboxylic acid (JMX1055)

390 mg, 97% in two steps. White solid. ¹H NMR (300 MHz CDCl₃) δ 7.74-7.67 (m, 2H), 7.27-7.19 (m, 2H).

### N-(4-Fluorophenyl)-5-((4-fluorophenyl)sulfonyl)-1,2,3-thiadiazole-4-carboxamide (JMX1061)

70 mg, 67% in two steps. White solid. ¹H NMR (300 MHz, CDCl₃) δ 9.24 (s, 1H), 8.37-8.27 (m, 2H), 7.68-7.58 (m, 2H), 7.31-7.21 (m, 2H), 7.13-7.02 (m, 2H). ¹³C NMR (75 MHz, CDCl₃) δ 166.8 (d, J=257.9 Hz), 163.1, 160.1 (d, J=244.0 Hz), 154.6, 152.0, 134.5 (d, J=3.0 Hz), 133.2 (d, J=10.1 Hz, 2C), 132.7 (d, J=3.0 Hz), 122.2 (d, J=8.0 Hz, 2C), 116.7 (d, J=22.7 Hz, 2C), 116.1 (d, J=22.6 Hz, 2C).

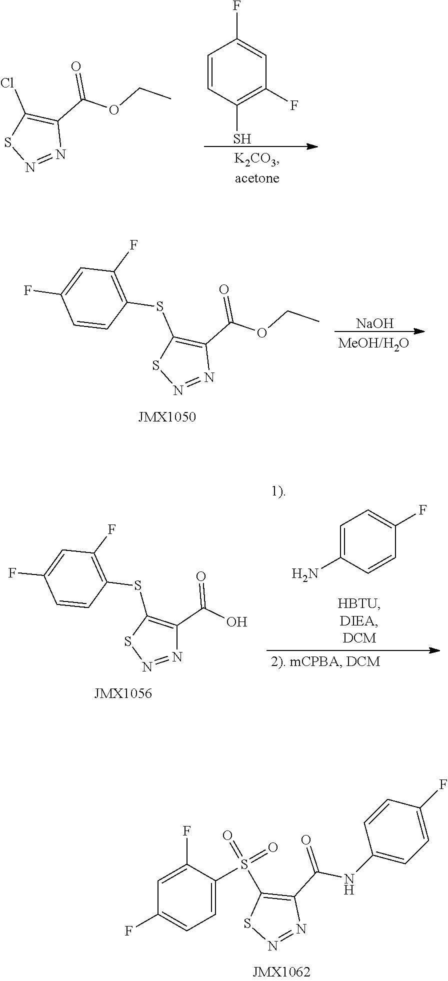

### Ethyl 5-((2,4-difluorophenyl)thio)-1,2,3-thiadiazole-4-carboxylate (JMX1050)

White solid. ¹H NMR (300 MHz, CDCl₃) δ 7.76-7.65 (m, 1H), 7.10-7.01 (m, 2H), 4.56 (q, J=7.2 Hz, 2H), 1.50 (t, J=7.2 Hz, 3H).

### 5-((2,4-Difluorophenyl)thio)-1,2,3-thiadiazole-4-carboxylic acid (JMX1056)

400 mg, 93% in two steps. White solid. ¹H NMR (300 MHz, CDCl₃) δ 7.77-7.67 (m, 1H), 7.12-7.02 (m, 2H).

### 5-((2,4-Difluorophenyl)sulfonyl)-N-(4-fluorophenyl)-1,2,3-thiadiazole-4-carboxamide (JMX1062)

62 mg, 58% in two steps. Grey solid. ¹H NMR (300 MHz, CDCl₃) δ 9.24 (s, 1H), 8.47-8.37 (m, 1H), 7.64-7.47 (m, 2H), 7.20-7.10 (m, 1H), 7.08-6.98 (m, 2H), 6.94-6.84 (m, 1H). ¹³C NMR (75 MHz, CDCl₃) δ 167.5 (dd, J=259.9, 11.7 Hz), 161.8 (d, J=16.4 Hz), 160.5 (dd, J=258.8, 13.4 Hz), 158.5, 154.5, 152.3, 135.1 (d, J=11.1 Hz), 132.6 (d, J=2.9 Hz), 123.4 (dd, J=12.5, 3.8 Hz), 122.1 (d, J=8.0 Hz, 2C), 116.0 (d, J=22.5 Hz, 2C), 112.4 (dd, J=22.1, 3.5 Hz), 105.7 (dd, J=26.2, 24.1 Hz).

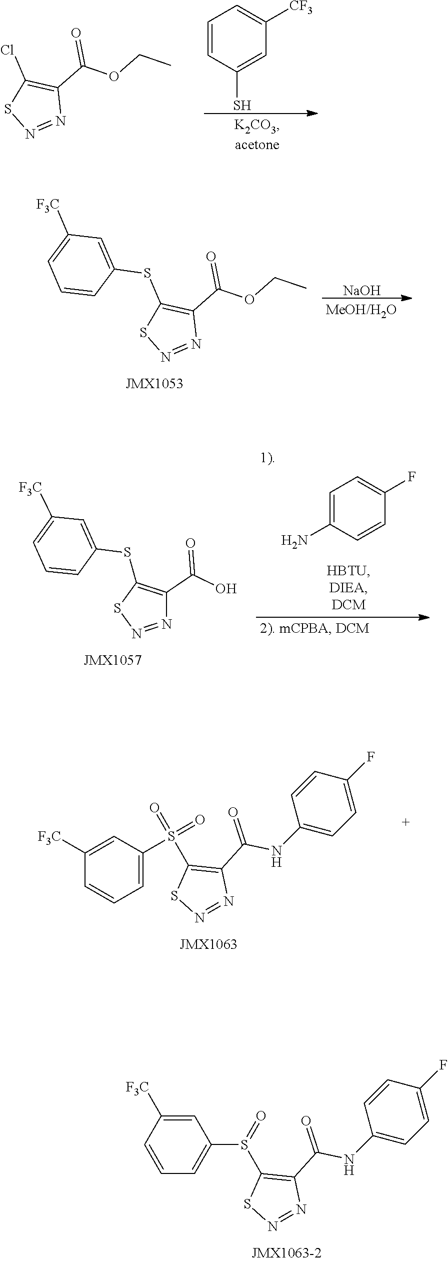

### Ethyl 5-((3-(trifluoromethyl)phenyl)thio)-1,2,3-thiadiazole-4-carboxylate (JMX1053)

White solid. ¹H NMR (300 MHz, CDCl₃) δ 7.97 (s, 1H), 7.90 (d, J=7.8 Hz, 1H), 7.84 (d, J=7.8 Hz, 1H), 7.68 (t, J=7.8 Hz, 1H), 4.56 (q, J=7.2 Hz, 2H), 1.50 (t, J=7.2 Hz, 3H).

### 5-((3-(Trifluoromethyl)phenyl)thio)-1,2,3-thiadiazole-4-carboxylic acid (JMX1057)

410 mg, 86% in two steps. ¹H NMR (300 MHz, CDCl₃) δ 7.99 (s, 1H), 7.92 (d, J=7.8 Hz, 1H), 7.86 (d, J=8.1 Hz, 1H), 7.70 (t, J=7.8 Hz, 1H).

### N-(4-Fluorophenyl)-5-((3-(trifluoromethyl)phenyl)sulfonyl)-1,2,3-thiadiazole-4-carboxamide (JMX1063)

53 mg, 53% in two steps. Tan solid. ¹H NMR (300 MHz, CDCl₃) δ 9.23 (s, 1H), 8.55-8.49 (m, 2H), 7.98-7.91 (m, 1H), 7.80-7.72 (m, 1H), 7.66-7.57 (m, 2H), 7.12-7.02 (m, 2H). ¹³C NMR (75 MHz, CDCl₃) δ 162.3, 160.1 (d, J=244.0 Hz), 154.4, 152.3, 139.7, 133.5, 132.7 (d, J=2.9 Hz), 132.0 (q, J=33.8 Hz), 131.8 (q, J=3.5 Hz), 130.1, 127.3 (q, J=3.8 Hz), 123.0 (q, J=271.4 Hz), 122.0 (d, J=8.0 Hz, 2C), 116.1 (d, J=22.6 Hz, 2C).

### N-(4-Fluorophenyl)-5-((3-(trifluoromethyl)phenyl)sulfinyl)-1,2,3-thiadiazole-4-carboxamide (JMX1063-2)

24 mg, 25% in two steps. Beige solid. ¹H NMR (300 MHz, CDCl₃) δ 9.20 (s, 1H), 8.37 (s, 1H), 8.28 (d, J=7.8 Hz, 1H), 7.77 (d, J=7.8 Hz, 1H), 7.70-7.60 (m, 3H), 7.17-7.07 (m, 2H). ¹³C NMR (75 MHz, CDCl₃) δ 174.4, 160.3 (d, J=244.2 Hz), 156.2, 151.6, 145.5, 132.6 (d, J=2.9 Hz), 132.4 (q, J=33.3 Hz), 130.4, 129.3 (q, J=3.5 Hz), 129.1, 123.3 (q, J=271.4 Hz), 122.7 (q, J=3.8 Hz), 122.2 (d, J=8.0 Hz, 2C), 116.3 (d, J=22.7 Hz, 2C).

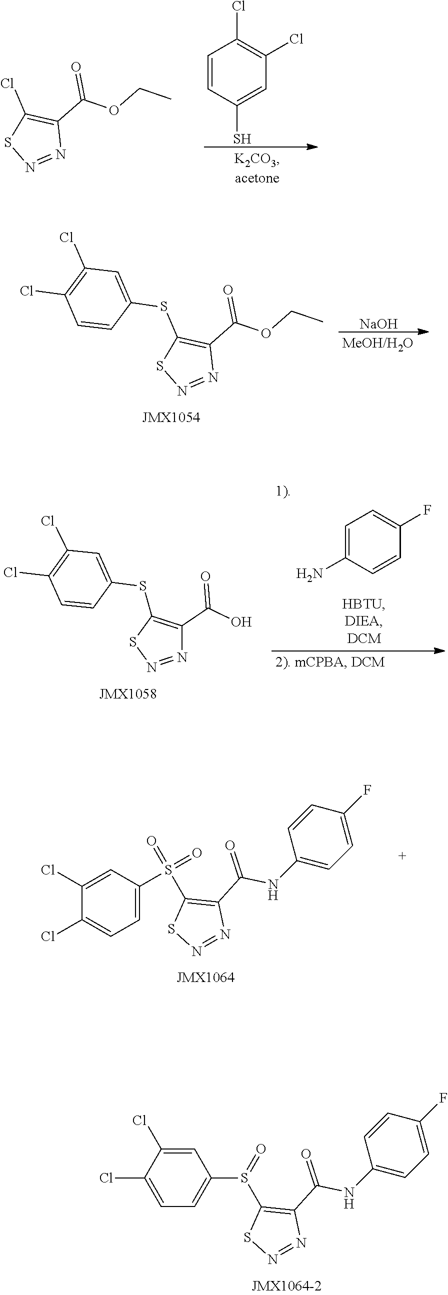

### Ethyl 5-((3,4-dichlorophenyl)thio)-1,2,3-thiadiazole-4-carboxylate (JMX1054)

White solid. ¹H NMR (300 MHz, CDCl₃) δ 7.97 (s, 1H), 7.90 (d, J=7.8 Hz, 1H), 7.84 (d, J=7.8 Hz, 1H), 7.68 (t, J=7.8 Hz, 1H), 4.56 (q, J=7.2 Hz, 2H), 1.50 (t, J=7.2 Hz, 3H).

### 5-((3,4-Dichlorophenyl)thio)-1,2,3-thiadiazole-4-carboxylic acid (JMX1058)

440 mg, 92% in two steps. White solid. ¹H NMR (300 MHz, DMSO-d₆) δ 14.16 (s, 1H), 8.21 (d, J=1.8 Hz, 1H), 7.88 (d, J=8.4 Hz, 1H), 7.83 (dd, J=8.4, 2.1 Hz, 1H).

### 5-((3,4-Dichlorophenyl)sulfonyl)-N-(4-fluorophenyl)-1,2,3-thiadiazole-4-carboxamide (JMX1064)

54 mg, 55%. Yellow solid. ¹H NMR (300 MHz, CDCl₃) δ 9.23 (s, 1H), 8.30 (d, J=2.1 Hz, 1H), 8.15 (dd, J=8.4, 2.1 Hz, 1H), 7.72-7.56 (m, 3H), 7.15-7.01 (m, 2H). ¹³C NMR (75 MHz, CDCl₃) δ 162.2, 160.2 (d, J=244.1 Hz), 154.4, 152.2, 140.6, 138.0, 134.0, 132.6 (d, J=2.9 Hz), 131.7, 131.3, 129.2, 122.1 (d, J=8.0 Hz, 2C), 116.2 (d, J=22.6 Hz, 2C).

### 5-((3,4-Dichlorophenyl)sulfinyl)-N-(4-fluorophenyl)-1,2,3-thiadiazole-4-carboxamide (JMX1064-2)

9 mg, 9% in two steps. Beige solid. ¹H NMR (300 MHz, CDCl₃) δ 9.18 (s, 1H), 8.14 (d, J=2.1 Hz, 1H), 7.92 (dd, J=8.4, 2.1 Hz, 1H), 7.70-7.62 (m, 2H), 7.58 (d, J=8.5 Hz, 1H), 7.17-7.07 (m, 2H). ¹³C NMR (75 MHz, CDCl₃) δ 174.2, 160.3 (d, J=244.4 Hz), 156.2, 151.5, 143.8, 137.4, 134.6, 132.5 (d, J=2.9 Hz), 131.7, 127.3, 125.0, 122.2 (d, J=8.0 Hz, 2C), 116.3 (d, J=22.6 Hz, 2C).

### Example 14—Inhibitors for Cryptococcal Fungi by Targeting the Prp8 Intein

Materials and Methods: Cloning, expression and purification—The split green fluorescence protein GFP-Prp8 and split Renilla luciferase RLuc-Prp8 were constructed, expressed, and purified as described previously. Li et al., “Cisplatin Protects Mice From Challenge of Cryptococcus neoformans by Targeting the Prp8 Intein,” Emerg. Microbes. Infect. 8:895-908 (2019) and Li et al., “Small-Molecule Inhibitors for the Prp8 Intein as Antifungal Agents,” Proc. Natl. Acad. Sci. USA 118 (2021), both of which are hereby incorporated by reference in their entirety.

The constructs for Cys-free (Cf) GFP-Prp8 and Cys-free nanoluciferase NnLuc-Prp8 were codon-optimized, synthesized, and cloned into the pET28a vector using the NcoI and XhoI sites by Gene Universal (Gene Universal, Newark Del.). For CfGFP-Prp8, the gene insert encoding the Prp8 intein of Cryptococcus gattii was inserted between residues 128 and 129 of a CfGFP. Suzuki et al., “Development of Cysteine-Free Fluorescent Proteins for the Oxidative Environment,” PLoS One 7:e37551 (2012), which is hereby incorporated by reference in its entirety. Within the Prp8 intein, a His6-tag and a Myc-tag with the sequence GHHHHHHEQKLISEEDLG (SEQ ID NO: 7) was inserted between the Prp8 residues 132 and 133 to facilitate purification and identification. For NnLuc-Prp8, the DNA fragment encoding the same His₆-Myc-Prp8 fragment was inserted between residues 157 and 158 of a Cys-free nanoluciferase. Dixon et al., “NanoLuc Complementation Reporter Optimized for Accurate Measurement of Protein Interactions in Cells,” ACS Chem. Biol. 11:400-08 (2016), which is hereby incorporated by reference in its entirety. The expression, refolding and purification of the CfGFP-Prp8 and NnLuc-Prp8 fusion proteins were carried out as described previously. Li et al., “Cisplatin Protects Mice From Challenge of Cryptococcus neoformans by Targeting the Prp8 Intein,” Emerg. Microbes. Infect. 8:895-908 (2019) and Li et al., “Small-Molecule Inhibitors for the Prp8 Intein as Antifungal Agents,” Proc. Natl. Acad. Sci. USA 118 (2021), both of which are hereby incorporated by reference in their entirety.

Split GFP-Prp8 and CJGFP-Prp8 intein splicing assays—Using black and clear bottom 96-well plates, the DPNB-treated, -refolded and -concentrated GFP-Prp8 or CfGFP-Prp8 fusion protein (0.5 μM) was incubated with compounds or Dimethylsulfoxide (DMSO) solvent control for 30 min at 25° C. in a 100 μl intein splicing buffer (20 mM Bis-tris propane, 0.5 M NaCl, 0.5 M arginine, 1 mM EDTA, and 0.05% CHAPS). Then 100 μl 2×TCEP was added to a final concentration of 20 μM. The reactions were incubated at 25° C. overnight. GFP-Prp8i splicing was monitored using a Bio-Tek FL800 microplate reader with excitation and emission wavelengths of 485 nm and 528 nm, respectively. Two readings were taken, one prior to addition of TCEP (I₀) and one after incubation overnight with TCEP (I_{f}). Fluorescence/luminescence differences (ΔFluorescence or ΔLuminescence) were defined as I_{f}−I₀ for each well. Relative GFP fluorescence-Prp8 (FLU) or RLuc-Prp8 luminescence (RLU, see below) was calculated by the following formula: % FLU (or % RLU)=(I_{f-comp}−I_{0-comp})/(I_{f-DMF}−I_{0-DMF})×100. DMSO control served as 100% splicing activity.

Split luciferase RLuc-Prp8 and NnLuc-Prp8 intein splicing assays—The split RLuc-Prp8 intein splicing assay was carried out and monitored using a Veritas luminometer, as described previously. Li et al., “Cisplatin Protects Mice From Challenge of Cryptococcus neoformans by Targeting the Prp8 Intein,” Emerg. Microbes. Infect. 8:895-908 (2019), which is hereby incorporated by reference in its entirety. For NnLuc-Prp8 intein splicing assay, the NnLuc-prp8 is diluted in assay buffer (50 mM Tris, 0.1% BSA, 1 mM EDTA, pH 7.4) and 50 ul is added to each well in 96 well black plates. Test molecules or the solvent was added and incubated for 30 to 60 min. A splicing inducer TCEP (50 ul, 100 μM) is added and incubated for 24 h. Luciferase substrate (Furimazine, 25 ul 1:10,000 diluted) is added and luminescence is read using a luminescence reader. IC₅₀ is calculated using Graphpad prism.

Cytotoxicity—Cytotoxicity assays were carried out using human lung carcinoma cell line A549, as described. Chen et al., “Selective Inhibition of the West Nile Virus Methyltransferase by Nucleoside Analogs,” Antiviral Res. 97:232-9 (2013), which is hereby incorporated by reference in its entirety. Briefly, the A549 cells (5×10³) were incubated with/without compounds in a 96-well plate at 37° C. for 48 h. Cell viability was determined using the MTT assay (ATCC) as was described previously (Chen et al., “Selective Inhibition of the West Nile Virus Methyltransferase by Nucleoside Analogs,” Antiviral Research 97(3):232-239 (2013) and Brecher et al., “Identification and Characterization of Novel Broad-Spectrum Inhibitors of the Flavivirus Methyltransferase,” ACS Infectious Diseases 1(8):340-349 (2015), both of which are hereby incorporated by reference in their entirety). N=3.

Fungal susceptibility test—The fungal susceptibility test was carried out with a compound concentration series as described. Li et al., “Cisplatin Protects Mice From Challenge of Cryptococcus neoformans by Targeting the Prp8 Intein,” Emerg. Microbes. Infect. 8:895-908 (2019), which is hereby incorporated by reference in its entirety. The broth microdilution method was used to determine the compound MIC values against C. neoformans. In brief, serial 2-fold dilutions of the compounds were made in 100 μl RPMI-1640 medium with MOPS in 96-well U-bottom plates. The C. neoformans H99 strain (100 μl of 0.5×10⁵/ml) in log phase was added to each well. After incubating the cells at 30° C. for 48 h, the cell culture was visualized to determine MIC. The MIC break-point was defined as no visible fungal growth by naked eye or by determining the 90% growth inhibition using absorbance at 630 nm, compared with no inhibitor control in a 96-well plate.

Results—54 derivatives of 6G-318S, an inhibitor for the Prp8 intein of pathogenic fungi, Cryptococcus neoformans and C. gattii were synthesized (Table 4). Using the split green fluorescence protein (GFP) (or Cys-free (Cf) GFP) and split Renilla luciferase (or Nanoluciferase (NnLuc)) assays, the inhibition efficacy of these derivatives was measured against the Prp8 intein of C. gattii. All derivatives inhibited the splicing of the GFP/CfGFP-Prp8 and RLuc/NnLuc-Prp8 intein with various potency (IC₅₀).

TABLE 4
IC₅₀, EC₅₀, and CC₅₀ (all in μM) of 6G-318S Derivatives

| | | IC₅₀ (μM) | | | MIC | |
|---|---|---|---|---|---|---|
| Formula | | GFP/CfGFP- | RLuc/NnLuc- | CC50 | (μM) | |
| (Comp ID) | Structure | Prp8 | Prp8 | (μM) | H99 | TI^{e} |
| Ia′ (6G-318S) | 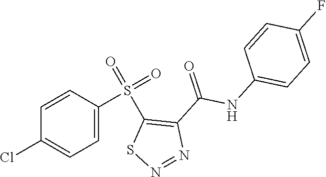 [SMILES: O=C(Nc1ccc(F)cc1)c1nnsc1S(=O)(=O)c1ccc(Cl)cc1] | 5.8^{a}/21.4^{b} | 11.2^{c}/16.2^{d} | 35 | 1.56 | 22.4 |
| Ia (JMX0515) | 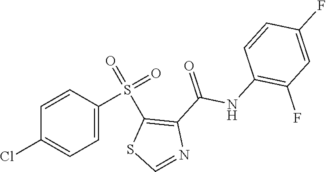 [SMILES: O=C(Nc1ccc(F)cc1F)c1ncsc1S(=O)(=O)c1ccc(Cl)cc1] | >50 | 37.7 | 210 | >100 | <2.1 |
| Ib (JMX0516) | 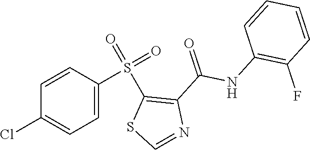 [SMILES: O=C(Nc1ccccc1F)c1ncsc1S(=O)(=O)c1ccc(Cl)cc1] | 49.3 | 13.3 | 144.4 | >100 | <1.4 |
| Ic (JMX0517) | 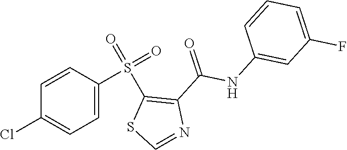 [SMILES: O=C(Nc1cccc(F)c1)c1ncsc1S(=O)(=O)c1ccc(Cl)cc1] | >50 | 28.4 | 160.6 | >100 | <1.6 |
| Id (JMX0518) | 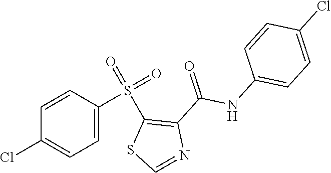 [SMILES: O=C(Nc1ccc(Cl)cc1)c1ncsc1S(=O)(=O)c1ccc(Cl)cc1] | 28.0 | 6.5 | 57.3 | 50 | 1.1 |
| Ie (JMX0519) | 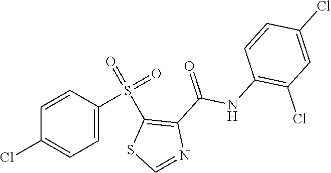 [SMILES: O=C(Nc1ccc(Cl)cc1Cl)c1ncsc1S(=O)(=O)c1ccc(Cl)cc1] | 50.0 | 11.7 | 245.2 | >100 | <2.4 |
| If (JMX0520) | 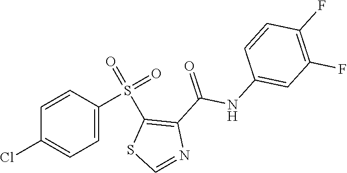 [SMILES: O=C(Nc1ccc(F)c(F)c1)c1ncsc1S(=O)(=O)c1ccc(Cl)cc1] | >50 | 27.5 | 81.6 | >100 | <0.8 |
| Ig (JMX0521) | 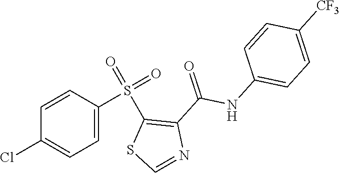 [SMILES: O=C(Nc1ccc(C(F)(F)F)cc1)c1ncsc1S(=O)(=O)c1ccc(Cl)cc1] | 50.0 | 29.3 | 54.1 | >100 | <0.5 |
| Ih (JMX0522) | 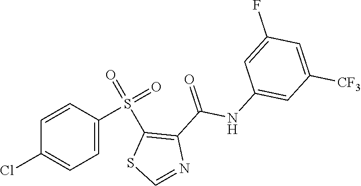 [SMILES: O=C(Nc1cc(F)cc(C(F)(F)F)c1)c1ncsc1S(=O)(=O)c1ccc(Cl)cc1] | 23.5 | 20.2 | 97.7 | 100 | 1.0 |
| Ii (JMX0523) | 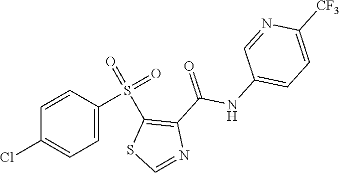 [SMILES: O=C(Nc1ccc(C(F)(F)F)nc1)c1ncsc1S(=O)(=O)c1ccc(Cl)cc1] | >50 | 37.1 | 113.7 | >100 | <1.1 |
| Ij (JMX0524) | 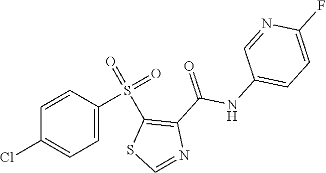 [SMILES: O=C(Nc1ccc(F)nc1)c1ncsc1S(=O)(=O)c1ccc(Cl)cc1] | >50 | 7.8 | 174.2 | >100 | <1.7 |
| Ik (JMX0525) | 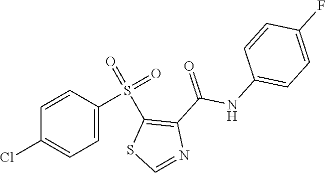 [SMILES: O=C(Nc1ccc(F)cc1)c1ncsc1S(=O)(=O)c1ccc(Cl)cc1] | >50 | 6.4 | 309.4 | >100 | <3.1 |
| Il (JMX0541) | 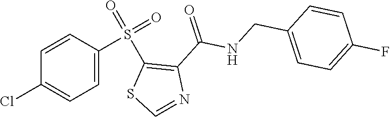 [SMILES: O=C(NCc1ccc(F)cc1)c1ncsc1S(=O)(=O)c1ccc(Cl)cc1] | >50 | 10.6 | 113.9 | 100 | 1.1 |
| Im (JMX0564) | 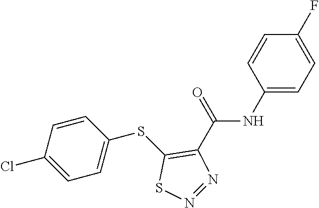 [SMILES: O=C(Nc1ccc(F)cc1)c1nnsc1Sc1ccc(Cl)cc1] | 55.7 | 15.8 | 418.0 | >100 | <4.2 |
| In (JMX0565-1) | 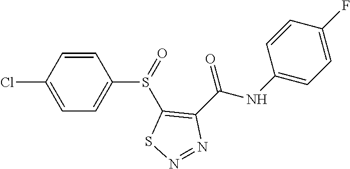 [SMILES: O=C(Nc1ccc(F)cc1)c1nnsc1S(=O)c1ccc(Cl)cc1] | 7.1 | 16.6 | 32.5 | 0.097 | 335 |
| Io (JMX0569) | 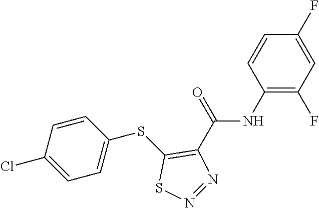 [SMILES: O=C(Nc1ccc(F)cc1F)c1nnsc1Sc1ccc(Cl)cc1] | >50 | 36.2 | 186.1 | >100 | <1.9 |
| Ip (JMX0570) | 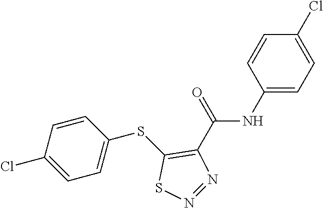 [SMILES: O=C(Nc1ccc(Cl)cc1)c1nnsc1Sc1ccc(Cl)cc1] | 38.7 | 21.6 | 83.7 | >100 | <0.8 |
| Iq (JMX0571) | 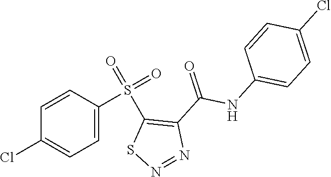 [SMILES: O=C(Nc1ccc(Cl)cc1)c1nnsc1S(=O)(=O)c1ccc(Cl)cc1] | 1.8 | 12.6 | 33.8 | 0.39 | 86.5 |
| Ir (JMX0573) | 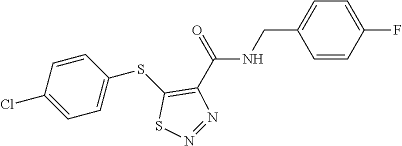 [SMILES: O=C(NCc1ccc(F)cc1)c1nnsc1Sc1ccc(Cl)cc1] | >50 | 5.5 | 282.5 | >100 | <2.8 |
| Is (JMX0574) | 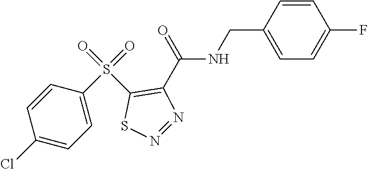 [SMILES: O=C(NCc1ccc(F)cc1)c1nnsc1S(=O)(=O)c1ccc(Cl)cc1] | 1.6 | 59.1 | 21.5 | 0.78 | 27.5 |
| It (JMX0575) | 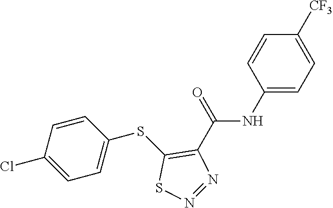 [SMILES: O=C(Nc1ccc(C(F)(F)F)cc1)c1nnsc1Sc1ccc(Cl)cc1] | >50 | 21.5 | 271.4 | >100 | <2.7 |
| Iu (JMX0576) | 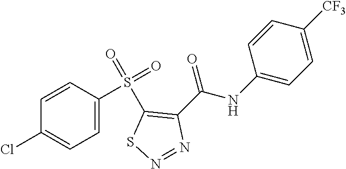 [SMILES: O=C(Nc1ccc(C(F)(F)F)cc1)c1nnsc1S(=O)(=O)c1ccc(Cl)cc1] | 0.9 | 11.9 | 40.3 | 0.78 | 51.7 |
| Iv (JMX0580) | 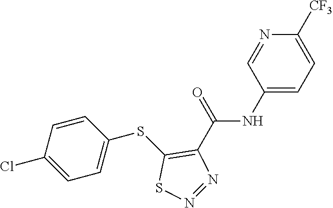 [SMILES: O=C(Nc1ccc(C(F)(F)F)nc1)c1nnsc1Sc1ccc(Cl)cc1] | 35.3 | 6.4 | 33.1 | >100 | <0.3 |
| Iw (JMX0581) | 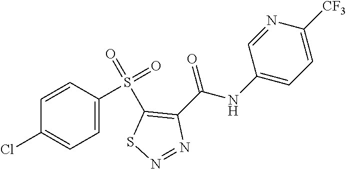 [SMILES: O=C(Nc1ccc(C(F)(F)F)nc1)c1nnsc1S(=O)(=O)c1ccc(Cl)cc1] | 6.1 | 11.2 | 92.4 | 0.78 | 118 |
| Ix (JMX0584) | 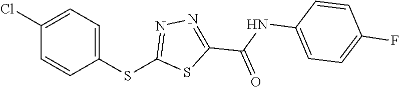 [SMILES: O=C(Nc1ccc(F)cc1)c1nnc(Sc2ccc(Cl)cc2)s1] | >50 | 11.0 | >150 | >100 | <1.5 |
| Iy (JMX0585-1) | 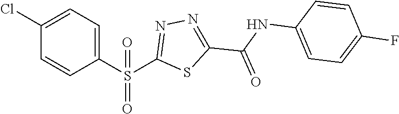 [SMILES: O=C(Nc1ccc(F)cc1)c1nnc(S(=O)(=O)c2ccc(Cl)cc2)s1] | 5.7 | 19.4 | 26.0 | 3.12 | 8.3 |
| Iz (JMX0585-2) | 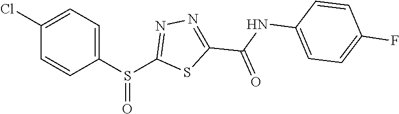 [SMILES: O=C(Nc1ccc(F)cc1)c1nnc(S(=O)c2ccc(Cl)cc2)s1] | 4.5 | 20.9 | 28.5 | 0.097 | 293.7 |
| Ic′ (JMX 871) | 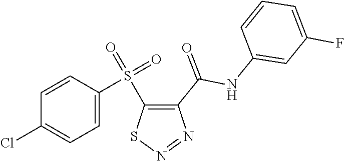 [SMILES: O=C(Nc1cccc(F)c1)c1nnsc1S(=O)(=O)c1ccc(Cl)cc1] | 35.4 | 27.1 | 140.2 | 3.12 | 44.9 |
| Id′ (JMX872) | 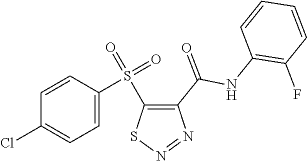 [SMILES: O=C(Nc1ccccc1F)c1nnsc1S(=O)(=O)c1ccc(Cl)cc1] | 28.3 | 31.8 | 161.9 | >50 | <3.2 |
| Ie′ (JMX876) | 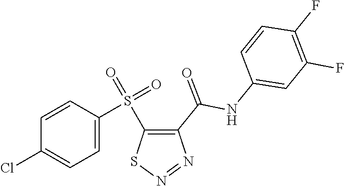 [SMILES: O=C(Nc1ccc(F)c(F)c1)c1nnsc1S(=O)(=O)c1ccc(Cl)cc1] | 33.5 | 22.0 | 162.7 | 3.12 | 52.1 |
| If′ (JMX1033) | 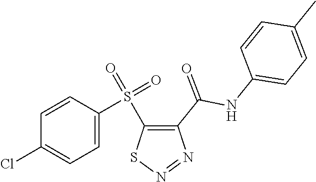 [SMILES: Cc1ccc(NC(=O)c2nnsc2S(=O)(=O)c2ccc(Cl)cc2)cc1] | 35.2 | 45.9 | 156.4 | >50 | <3.1 |
| Ig′ (JMX1033-2) | 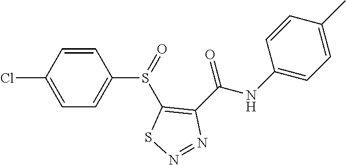 [SMILES: Cc1ccc(NC(=O)c2nnsc2S(=O)c2ccc(Cl)cc2)cc1] | 47.3 | 3.8 | 74.6 | >50 | <1.5 |
| Ih′ (JMX1034) | 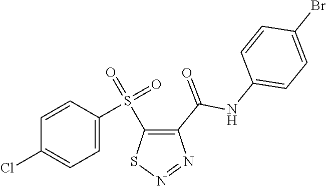 [SMILES: O=C(Nc1ccc(Br)cc1)c1nnsc1S(=O)(=O)c1ccc(Cl)cc1] | 33.1 | 15.4 | 84.8 | >50 | <1.7 |
| Ii′ (JMX1035) | 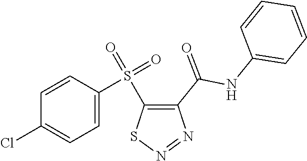 [SMILES: O=C(Nc1ccccc1)c1nnsc1S(=O)(=O)c1ccc(Cl)cc1] | 38.4 | 33.9 | 84.1 | 6.25 | 13.5 |
| Ij′ (JMX1036) | 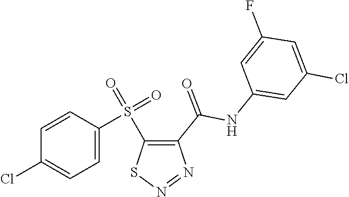 [SMILES: O=C(Nc1cc(F)cc(Cl)c1)c1nnsc1S(=O)(=O)c1ccc(Cl)cc1] | 32.9 | 21.4 | 70.9 | 1.56 | 45.4 |
| Ik′ (JMX1037) | 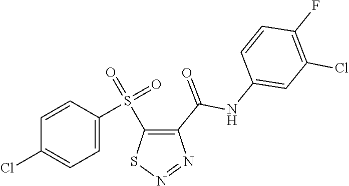 [SMILES: O=C(Nc1ccc(F)c(Cl)c1)c1nnsc1S(=O)(=O)c1ccc(Cl)cc1] | 31.4 | 14.1 | 63.2 | 25 | 2.5 |
| Il′ (JMX1038) | 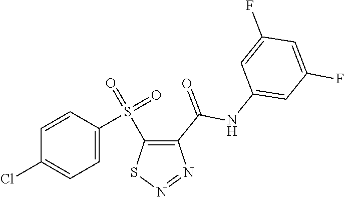 [SMILES: O=C(Nc1cc(F)cc(F)c1)c1nnsc1S(=O)(=O)c1ccc(Cl)cc1] | 37.1 | 37.4 | 54.2 | >50 | <1.1 |
| Im′ (JMX1040) | 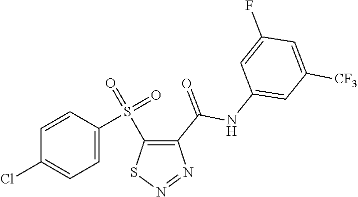 [SMILES: O=C(Nc1cc(F)cc(C(F)(F)F)c1)c1nnsc1S(=O)(=O)c1ccc(Cl)cc1] | 32.8 | 20.6 | 56.3 | 3.12 | 18.1 |
| In′ (JMX1041) | 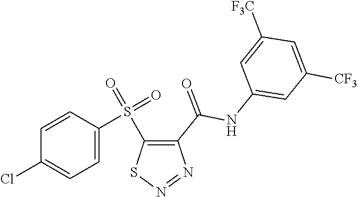 [SMILES: O=C(Nc1cc(C(F)(F)F)cc(C(F)(F)F)c1)c1nnsc1S(=O)(=O)c1ccc(Cl)cc1] | 37.9 | 18.5 | 66.7 | >50 | <1.3 |
| Io′ (JMX1042) | 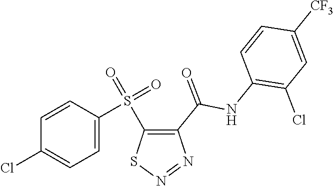 [SMILES: O=C(Nc1ccc(C(F)(F)F)cc1Cl)c1nnsc1S(=O)(=O)c1ccc(Cl)cc1] | 41.5 | 10.9 | 98.7 | >50 | <2.0 |
| Ip′ (JMX1043) | 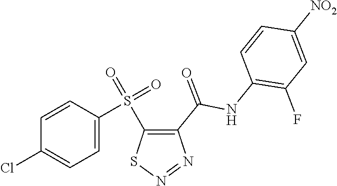 [SMILES: O=C(Nc1ccc([N+](=O)[O-])cc1F)c1nnsc1S(=O)(=O)c1ccc(Cl)cc1] | 38.1 | 19.6 | 66.9 | >50 | <1.3 |
| Iq′ (JMX1044) | 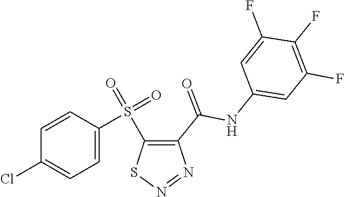 [SMILES: O=C(Nc1cc(F)c(F)c(F)c1)c1nnsc1S(=O)(=O)c1ccc(Cl)cc1] | 31.8 | 16.3 | 47.8 | >50 | 1.0 |
| Ir′ (JMX1044-2) | 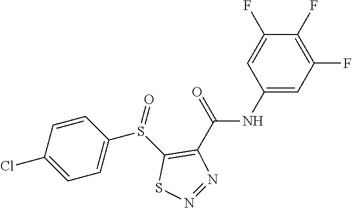 [SMILES: O=C(Nc1cc(F)c(F)c(F)c1)c1nnsc1S(=O)c1ccc(Cl)cc1] | 26.9 | 4.4 | 47.5 | >50 | 1.0 |
| Is′ (JMX1045) | 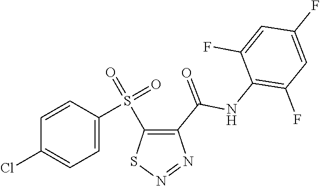 [SMILES: O=C(Nc1c(F)cc(F)cc1F)c1nnsc1S(=O)(=O)c1ccc(Cl)cc1] | 34.3 | 15.9 | 47.9 | >50 | 1.0 |
| It′ (JMX1045-2) | 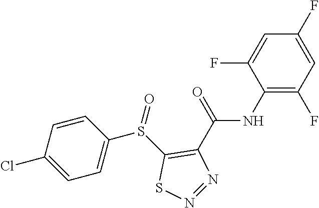 [SMILES: O=C(Nc1c(F)cc(F)cc1F)c1nnsc1S(=O)c1ccc(Cl)cc1] | 27.4 | 8.6 | 61.0 | 3.12 | 19.6 |
| Iu′ (JMX1046) | 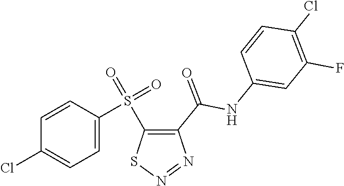 [SMILES: O=C(Nc1ccc(Cl)c(F)c1)c1nnsc1S(=O)(=O)c1ccc(Cl)cc1] | 34.1 | 20.1 | 145.4 | >50 | <2.9 |
| Iv′ (JMX1046-2) | 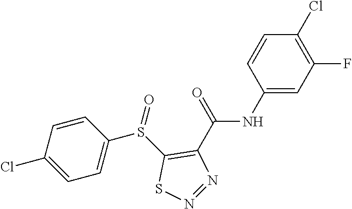 [SMILES: O=C(Nc1ccc(Cl)c(F)c1)c1nnsc1S(=O)c1ccc(Cl)cc1] | 32.2 | 7.5 | 149.3 | 1.56 | 95.7 |
| Iw′ (JMX1059) | 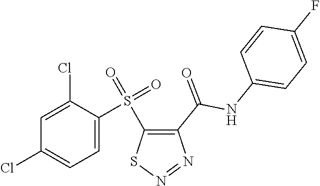 [SMILES: O=C(Nc1ccc(F)cc1)c1nnsc1S(=O)(=O)c1ccc(Cl)cc1Cl] | 31.9 | 20.9 | 68.1 | 12.5 | 5.4 |
| Ix′ (JMX1060) | 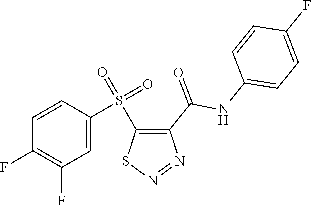 [SMILES: O=C(Nc1ccc(F)cc1)c1nnsc1S(=O)(=O)c1ccc(F)c(F)c1] | 32.8 | 23.5 | 61.1 | 50 | 1.2 |
| Iy′ (JMX1061) | 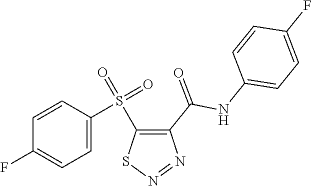 [SMILES: O=C(Nc1ccc(F)cc1)c1nnsc1S(=O)(=O)c1ccc(F)cc1] | 51.3 | 23.7 | 71.7 | >50 | <1.4 |
| Iz′ (JMX1062) | 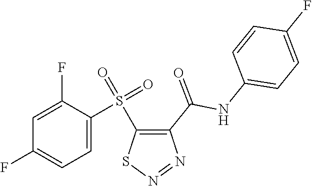 [SMILES: O=C(Nc1ccc(F)cc1)c1nnsc1S(=O)(=O)c1ccc(F)cc1F] | 38.2 | 21.7 | 65.4 | >50 | <1.3 |
| Ia″ (JMX1063) | 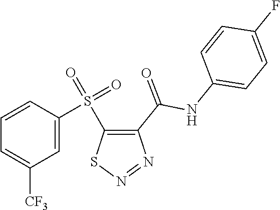 [SMILES: O=C(Nc1ccc(F)cc1)c1nnsc1S(=O)(=O)c1cccc(C(F)(F)F)c1] | 35.0 | 28.2 | 136.7 | 6.25 | 21.9 |
| Ib″ (JMX1063-2) | 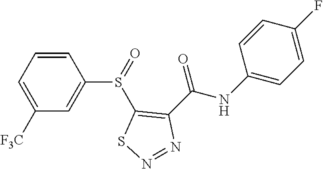 [SMILES: O=C(Nc1ccc(F)cc1)c1nnsc1S(=O)c1cccc(C(F)(F)F)c1] | 29.2 | 12.4 | 93.8 | 6.25 | 15.0 |
| Ic″ (JMX1064) | 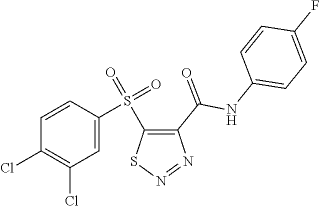 [SMILES: O=C(Nc1ccc(F)cc1)c1nnsc1S(=O)(=O)c1ccc(Cl)c(Cl)c1] | 32.6 | 26.0 | 78.4 | 1.56 | 50.1 |
| Id″ (JMX1064-2) | 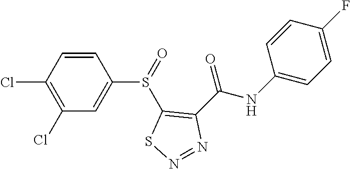 [SMILES: O=C(Nc1ccc(F)cc1)c1nnsc1S(=O)c1ccc(Cl)c(Cl)c1] | 19. 8 | 35.0 | 122.4 | >50 | <2.4 |

^{a}IC₅₀ was measured using split GFP-Prp8 assay. IC₅₀S for compounds from JMX0515 to JMX0585-2 were measured using split GFP-Prp8 assay.
^{b}IC₅₀ was measured using split Cys-free (Cf) GFP-Prp8. IC₅₀S for compounds from JMX0871 to JMX1064-2 were measured using split CfGFP-Prp8.
^{c}IC₅₀ was measured using split Renilla luciferase (RLuc)-Prp8 assay. IC₅₀S for compounds from JMX0515 to JMX0585-2 were measured using split RLuc-Prp8 assay.
^{d}IC₅₀ was measured using split nanoluciferase (NnLuc)-Prp8 assay. IC₅₀S for compounds from JMX0871 to JMX1064-2 were measured using split NnLuc-Prp8 assay.
^{e}TI: Therapeutic index defined as CC50/MIC.

To investigate the antifungal potency, the minimum inhibitory concentration (MIC) of these derivatives was measured against C. neoformans H99 strain, using microbroth dilution assay (Table 4). Among the 54 derivatives, compounds JMX0565-1, JMX0571, JMX0574, JMX0576, JMX0581, JMX0585-2, JMX1036, JMX1046-2, and JMX1064 showed antifungal activity against C. neoformans with potency equal to or better than the parent compound 6G-318S. The best compounds JMX0565-1 and JMX0585-2 showed much improved antifungal potency with MIC values of 0.097 μM towards C. neoformans.

The cytotoxicity (CC₅₀) of these compounds to the lung carcinoma cell line A549 (Table 4) was measured. These results showed that all the active compounds showed cytotoxicity profile similar to or better than the parent compound 6G-318S, leading to improved therapeutic index (TI) defined as CC₅₀/MIC. For the most potent compounds JMX0565-1 and JMX0585-2, the TI values are 335 and 294, respectively.

### CONCLUSION

Through structure-activity relationship synthesis, several 6G-318S derivatives were identified showing improved potency and therapeutic index, against C. neoformans.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1-7. (canceled)

8. A method, comprising:
administering to a subject a Prp8 intein splicing inhibitor of formula (I) 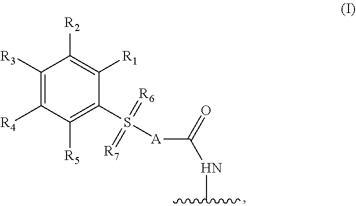
wherein R₁, R₂, R₃, R₄, and R₅ are independently selected from the group consisting of halogen, trifluoromethyl, hydrogen, amine, amide, nitrogen oxide, C₁-C₂₃ alkyl, aryl, heteroaryl, carbocycle, heterocycle, and oxygen, wherein one or more of the halogen, trifluoromethyl, amine, amide, nitrogen oxide, C₁-C₂₃ alkyl, aryl, heteroaryl, carbocycle, heterocycle, and oxygen optionally can be independently substituted with one or more halogen, hydrogen, C₁-C₃ alkyl, trifluoromethyl, or nitrogen oxide;
wherein R₆ and R₇ are independently selected from oxygen and hydrogen;
wherein A is independently selected from: 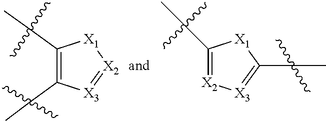
wherein 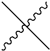
in A represent a point of attachment and wherein X₁, X₂, and X₃ are independently selected from carbon, nitrogen, sulfur, and oxygen; and
wherein [symbol] in formula (I) represents a point of attachment to at least one of: 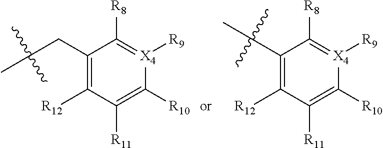
wherein X₄ is carbon or nitrogen;
wherein R₈, R₉, R₁₀, R₁₁, and R₁₂ are independently selected from hydrogen, halogen, trifluoromethyl, alkyl, and nitrogen oxide,
wherein the subject has an infection, or is at risk of developing an infection, with a fungus.

9. The method of claim 8, wherein (i) one or more of R₁, R₂, R₃, R₄, and R₅ is hydrogen, halogen, or trifluoromethyl; (ii) one or more of X₁, X₂, and X₃ is N or S; or both (i) and (ii).

10-13. (canceled)

14. The method of claim 8, wherein the Prp8 intein splicing inhibitor is a compound selected from formulae (Ia) through (Id″): 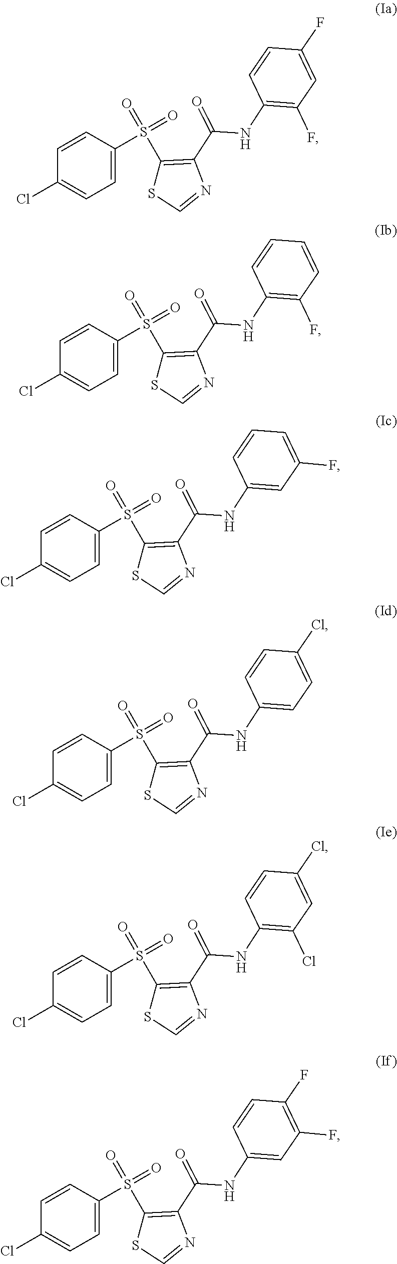 [SMILES: O=C(Nc1ccc(F)cc1F)c1ncsc1S(=O)(=O)c1ccc(Cl)cc1 ; O=C(Nc1ccccc1F)c1ncsc1S(=O)(=O)c1ccc(Cl)cc1 ; O=C(Nc1cccc(F)c1)c1ncsc1S(=O)(=O)c1ccc(Cl)cc1 ; O=C(Nc1ccc(Cl)cc1)c1ncsc1S(=O)(=O)c1ccc(Cl)cc1 ; O=C(Nc1ccc(Cl)cc1Cl)c1ncsc1S(=O)(=O)c1ccc(Cl)cc1 ; O=C(Nc1ccc(F)c(F)c1)c1ncsc1S(=O)(=O)c1ccc(Cl)cc1] 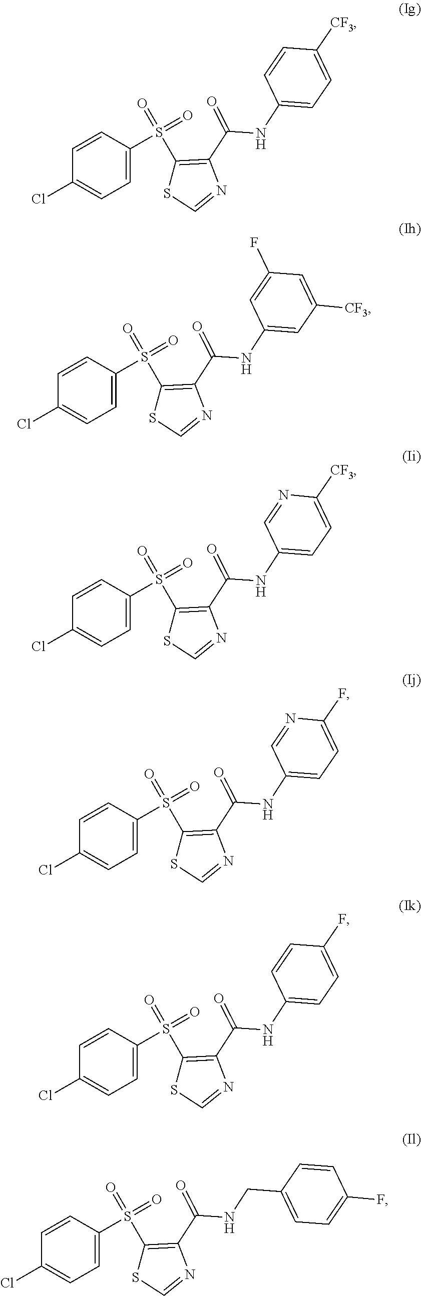 [SMILES: O=C(Nc1ccc(C(F)(F)F)cc1)c1ncsc1S(=O)(=O)c1ccc(Cl)cc1 ; O=C(Nc1cc(F)cc(C(F)(F)F)c1)c1ncsc1S(=O)(=O)c1ccc(Cl)cc1 ; O=C(Nc1ccc(C(F)(F)F)nc1)c1ncsc1S(=O)(=O)c1ccc(Cl)cc1 ; O=C(Nc1ccc(F)nc1)c1ncsc1S(=O)(=O)c1ccc(Cl)cc1 ; O=C(Nc1ccc(F)cc1)c1ncsc1S(=O)(=O)c1ccc(Cl)cc1 ; O=C(NCc1ccc(F)cc1)c1ncsc1S(=O)(=O)c1ccc(Cl)cc1] 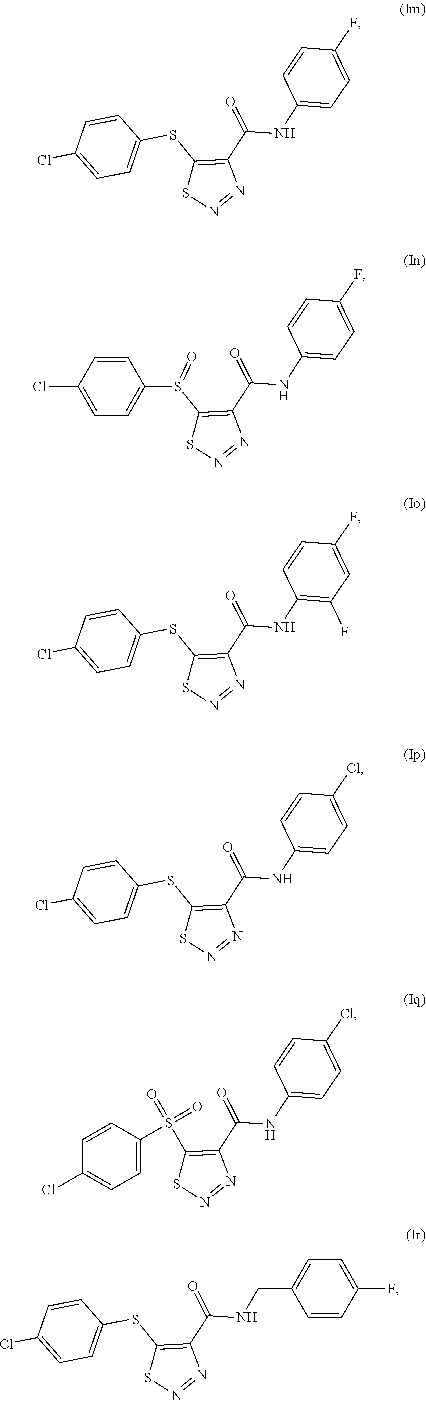 [SMILES: O=C(Nc1ccc(F)cc1)c1nnsc1Sc1ccc(Cl)cc1 ; O=C(Nc1ccc(F)cc1)c1nnsc1S(=O)c1ccc(Cl)cc1 ; O=C(Nc1ccc(F)cc1F)c1nnsc1Sc1ccc(Cl)cc1 ; O=C(Nc1ccc(Cl)cc1)c1nnsc1Sc1ccc(Cl)cc1 ; O=C(Nc1ccc(Cl)cc1)c1nnsc1S(=O)(=O)c1ccc(Cl)cc1 ; O=C(NCc1ccc(F)cc1)c1nnsc1Sc1ccc(Cl)cc1] 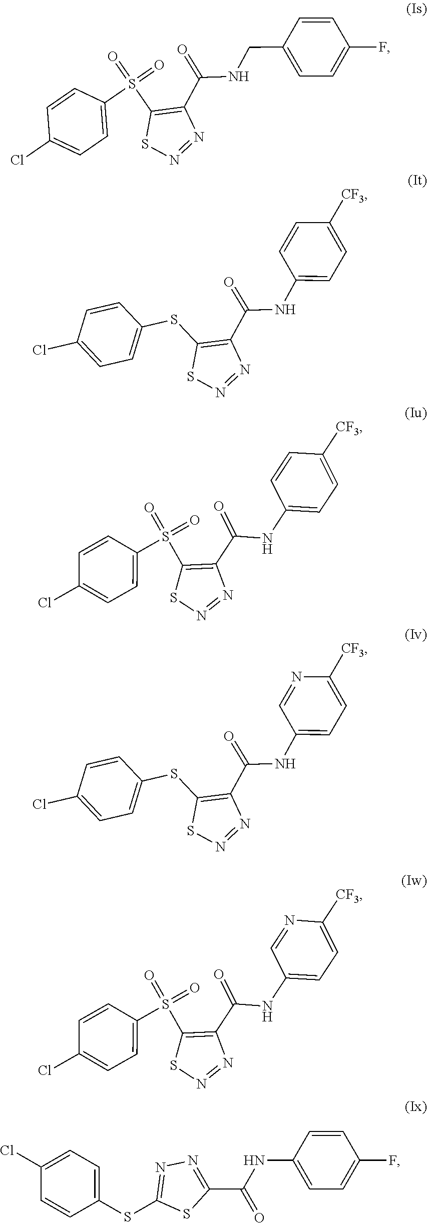 [SMILES: O=C(NCc1ccc(F)cc1)c1nnsc1S(=O)(=O)c1ccc(Cl)cc1 ; O=C(Nc1ccc(C(F)(F)F)cc1)c1nnsc1Sc1ccc(Cl)cc1 ; O=C(Nc1ccc(C(F)(F)F)cc1)c1nnsc1S(=O)(=O)c1ccc(Cl)cc1 ; O=C(Nc1ccc(C(F)(F)F)nc1)c1nnsc1Sc1ccc(Cl)cc1 ; O=C(Nc1ccc(C(F)(F)F)nc1)c1nnsc1S(=O)(=O)c1ccc(Cl)cc1 ; O=C(Nc1ccc(F)cc1)c1nnc(Sc2ccc(Cl)cc2)s1] 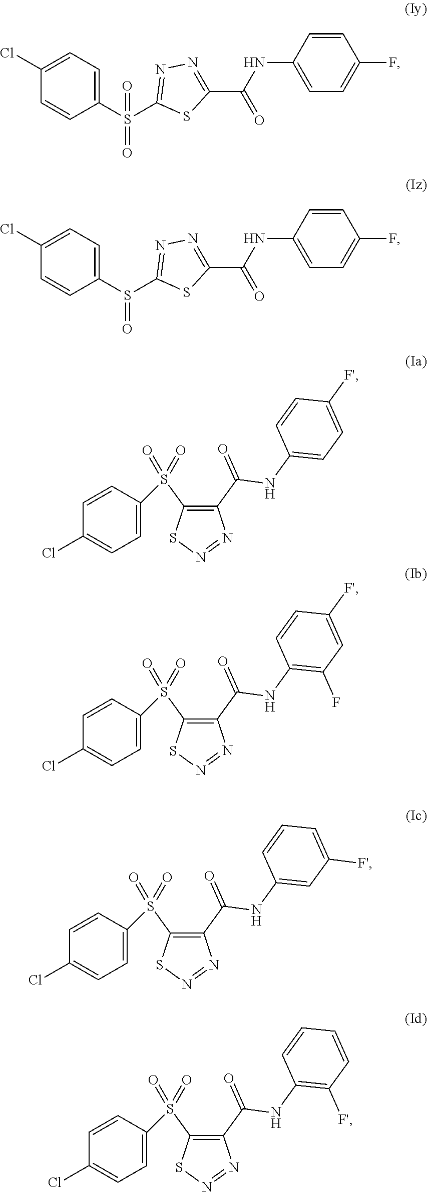 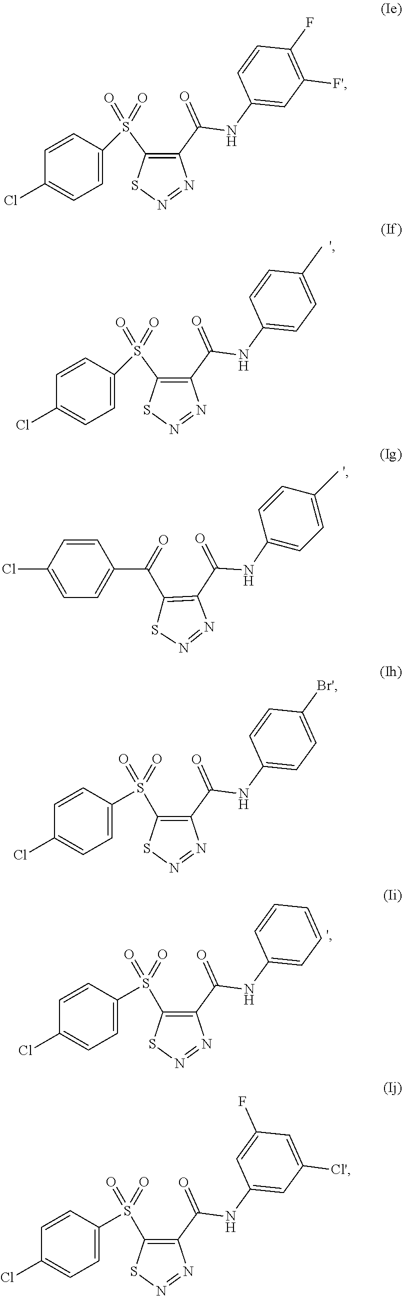 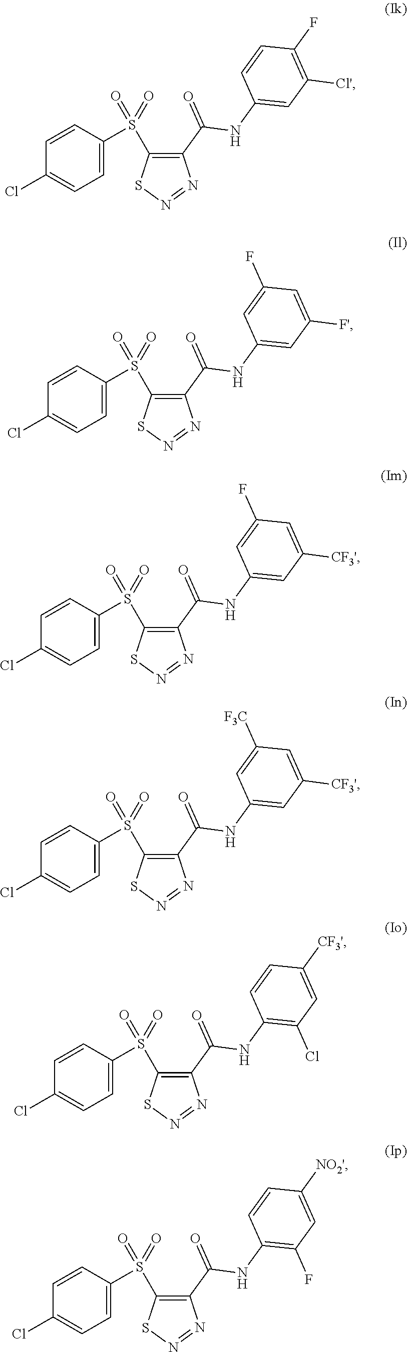 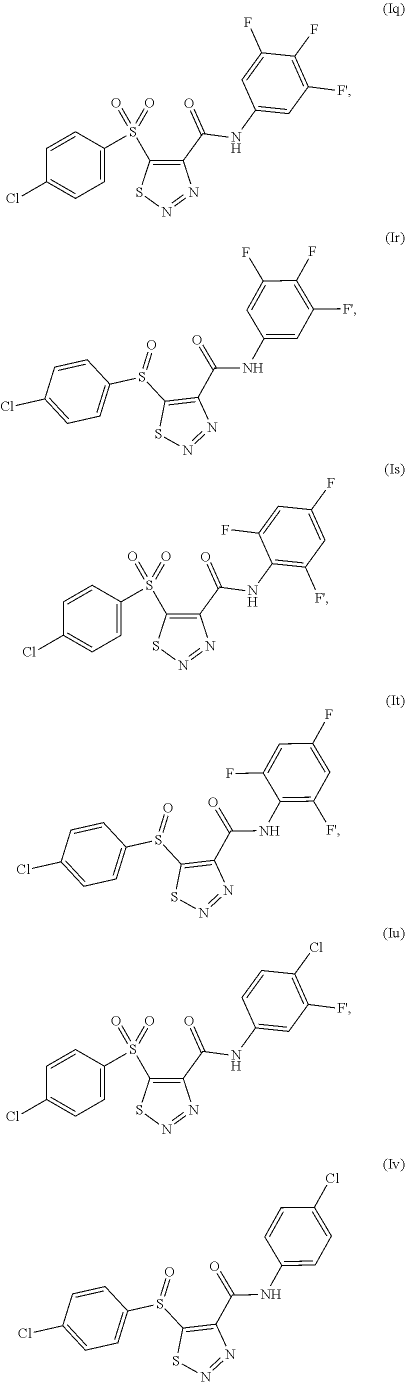 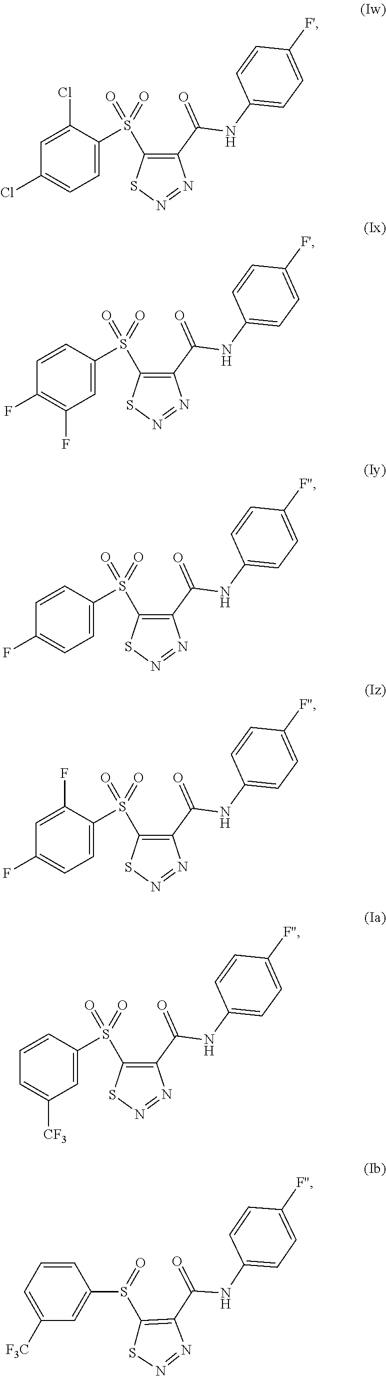 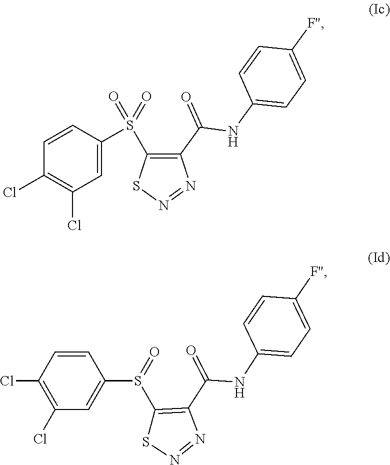
or a pharmaceutically acceptable salt thereof.

15. The method of claim 8, wherein said fungus is selected from one or more of Cryptococcus, Aspergillus, Blastomyces, Coccidiodes, Histoplasma, Phycomyces, Tinea corporis, Tinea unguis, Sporothrix schenckii, Pneumocystis carinii, and Candida.

16-17. (canceled)

18. The method of claim 8 further comprising:
administering one or more additional agents.

19. The method of claim 18, wherein said one or more additional agent is a compound selected from the group consisting of 5-fluorocytosine (5-FC), itraconazole, fluconazole, amphotericin B, anidulafungin, micafungin, caspofungin, posaconazole, and voriconazole.

20. (canceled)

21. The method of claim 8, wherein said treatment comprises:
administering said Prp8 intein splicing inhibitor to a subject transdermally, intradermally, parenterally, subcutaneously, intravenous injection, intra-arterial injection, intramuscular injection, intrapleurally, intraperitoneally, intrathecally, or by application to a mucous membrane, orally, by inhalation, by intranasal instillation, topically, or any combination thereof.

22. The method of claim 8 further comprising:
repeating said administering of said Prp8 intein splicing inhibitor.

23. The method of claim 8, wherein the subject is an infant, a juvenile, or an adult.

24-25. (canceled)

26. A method of inhibiting Prp8 intein expression or activity in a cell or tissue, said method comprising:
contacting the ell or tissue with a compound of formula (I): 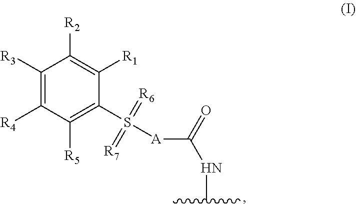
wherein R₁, R₂, R₃, R₄, and R₅ are independently selected from the group consisting of halogen, trifluoromethyl, hydrogen amine, amide, nitrogen oxide, C₁-C₂₃ alkyl, aryl, heteroaryl, carbocycle, heterocycle, and oxygen, wherein one or more of the halogen, trifluoromethyl, amine, amide, nitrogen oxide, C₁-C₂₃ alkyl, aryl, heteroaryl, carbocycle, heterocycle, and oxygen optionally can be independently substituted with one or more halogen, hydrogen, C₁-C₃ alkyl, trifluoromethyl, or nitrogen oxide;
wherein R₆ and R₇ are independently selected from oxygen and hydrogen;
wherein A is independently selected from: 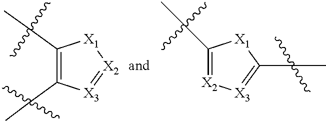
wherein 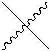
in A represent a point of attachment and wherein X₁, X₂, and X₃ are independently selected from carbon, nitrogen, sulfur, and oxygen; and
wherein [symbol] in formula (I) represents a point of attachment to at least one of: 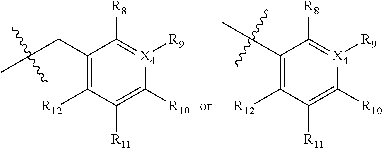
wherein X₄ is carbon or nitrogen;
wherein R₈, R₉, R₁₀, R₁₁, and R₁₂ are independently selected from hydrogen, halogen, trifluoromethyl, alkyl, and nitrogen oxide, under conditions effective to inhibit Prp8 intein expression or activity in a cell or tissue.

27. The method of claim 26, wherein (i) one or more of R₁, R₂, R₃, R₄, and R₅ is hydrogen, halogen, or trifluoromethyl; (ii) one or more of X₁, X₂, and X₃ is N or S; or both (i) and (ii).

28-31. (canceled)

32. The method of claim 26, wherein the Prp8 intein splicing inhibitor is a compound selected from formulae (Ia) through (Id″): 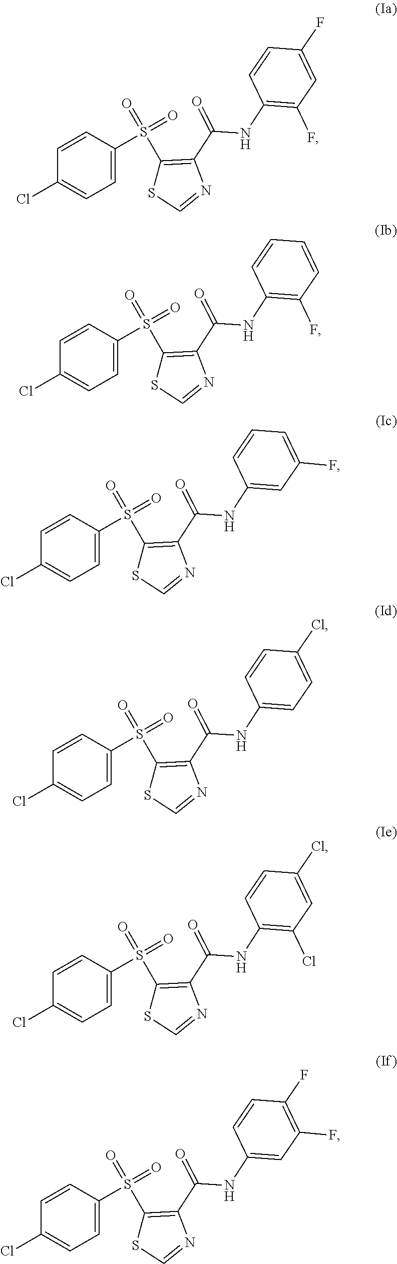 [SMILES: O=C(Nc1ccc(F)cc1F)c1ncsc1S(=O)(=O)c1ccc(Cl)cc1 ; O=C(Nc1ccccc1F)c1ncsc1S(=O)(=O)c1ccc(Cl)cc1 ; O=C(Nc1cccc(F)c1)c1ncsc1S(=O)(=O)c1ccc(Cl)cc1 ; O=C(Nc1ccc(Cl)cc1)c1ncsc1S(=O)(=O)c1ccc(Cl)cc1 ; O=C(Nc1ccc(Cl)cc1Cl)c1ncsc1S(=O)(=O)c1ccc(Cl)cc1 ; O=C(Nc1ccc(F)c(F)c1)c1ncsc1S(=O)(=O)c1ccc(Cl)cc1] 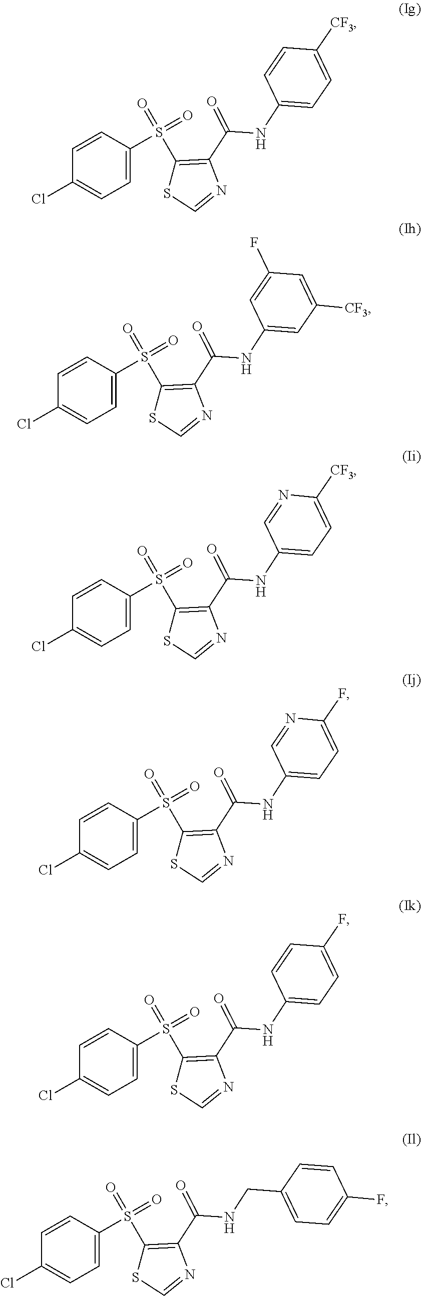 [SMILES: O=C(Nc1ccc(C(F)(F)F)cc1)c1ncsc1S(=O)(=O)c1ccc(Cl)cc1 ; O=C(Nc1cc(F)cc(C(F)(F)F)c1)c1ncsc1S(=O)(=O)c1ccc(Cl)cc1 ; O=C(Nc1ccc(C(F)(F)F)nc1)c1ncsc1S(=O)(=O)c1ccc(Cl)cc1 ; O=C(Nc1ccc(F)nc1)c1ncsc1S(=O)(=O)c1ccc(Cl)cc1 ; O=C(Nc1ccc(F)cc1)c1ncsc1S(=O)(=O)c1ccc(Cl)cc1 ; O=C(NCc1ccc(F)cc1)c1ncsc1S(=O)(=O)c1ccc(Cl)cc1] 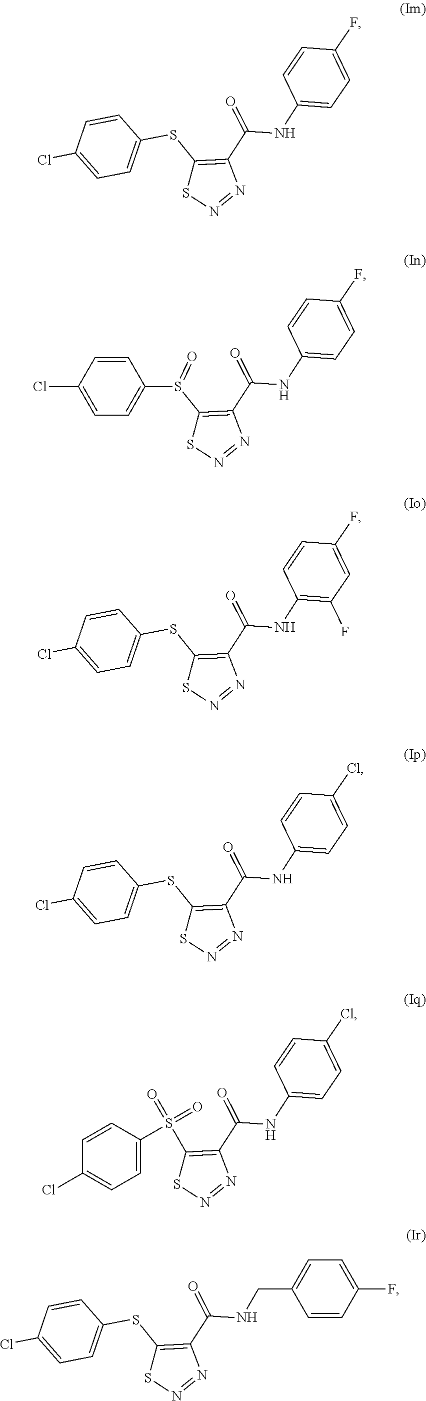 [SMILES: O=C(Nc1ccc(F)cc1)c1nnsc1Sc1ccc(Cl)cc1 ; O=C(Nc1ccc(F)cc1)c1nnsc1S(=O)c1ccc(Cl)cc1 ; O=C(Nc1ccc(F)cc1F)c1nnsc1Sc1ccc(Cl)cc1 ; O=C(Nc1ccc(Cl)cc1)c1nnsc1Sc1ccc(Cl)cc1 ; O=C(Nc1ccc(Cl)cc1)c1nnsc1S(=O)(=O)c1ccc(Cl)cc1 ; O=C(NCc1ccc(F)cc1)c1nnsc1Sc1ccc(Cl)cc1] 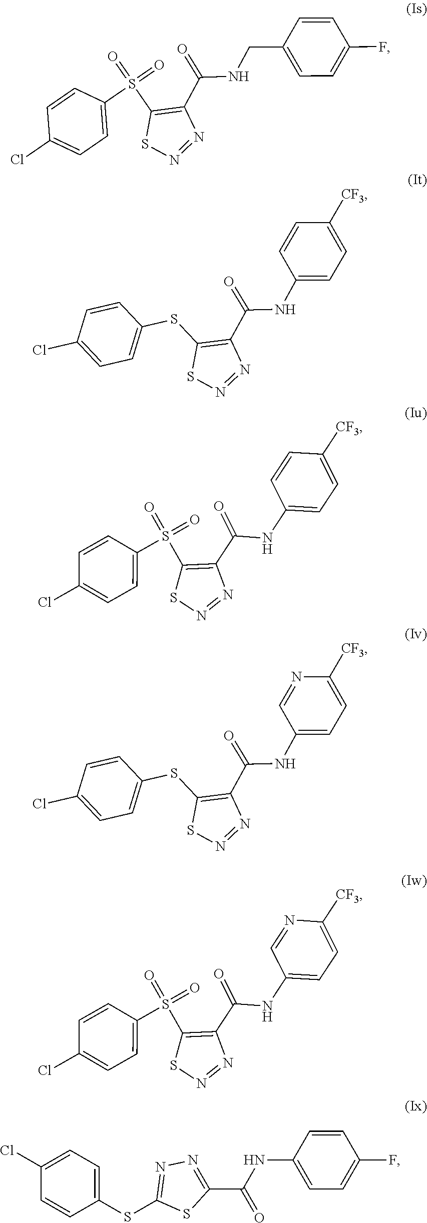 [SMILES: O=C(NCc1ccc(F)cc1)c1nnsc1S(=O)(=O)c1ccc(Cl)cc1 ; O=C(Nc1ccc(C(F)(F)F)cc1)c1nnsc1Sc1ccc(Cl)cc1 ; O=C(Nc1ccc(C(F)(F)F)cc1)c1nnsc1S(=O)(=O)c1ccc(Cl)cc1 ; O=C(Nc1ccc(C(F)(F)F)nc1)c1nnsc1Sc1ccc(Cl)cc1 ; O=C(Nc1ccc(C(F)(F)F)nc1)c1nnsc1S(=O)(=O)c1ccc(Cl)cc1 ; O=C(Nc1ccc(F)cc1)c1nnc(Sc2ccc(Cl)cc2)s1] 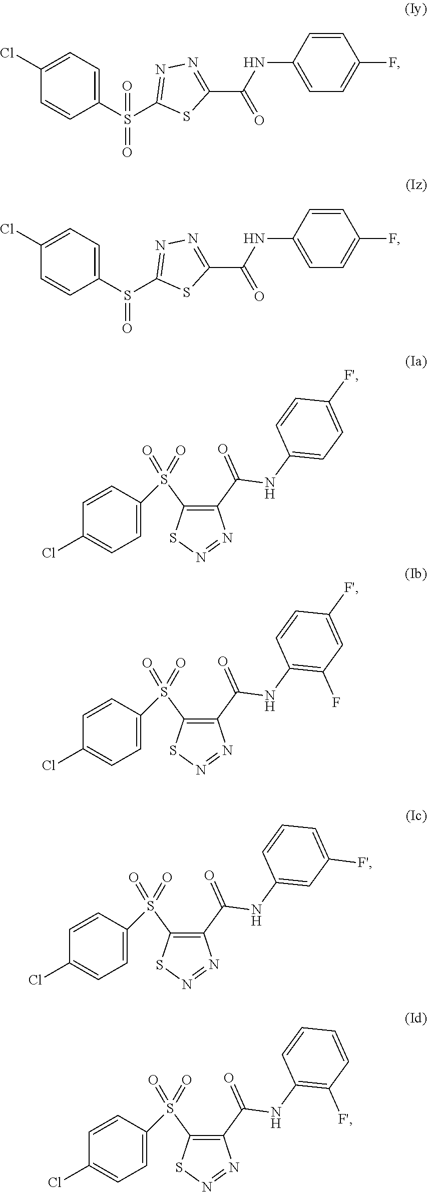 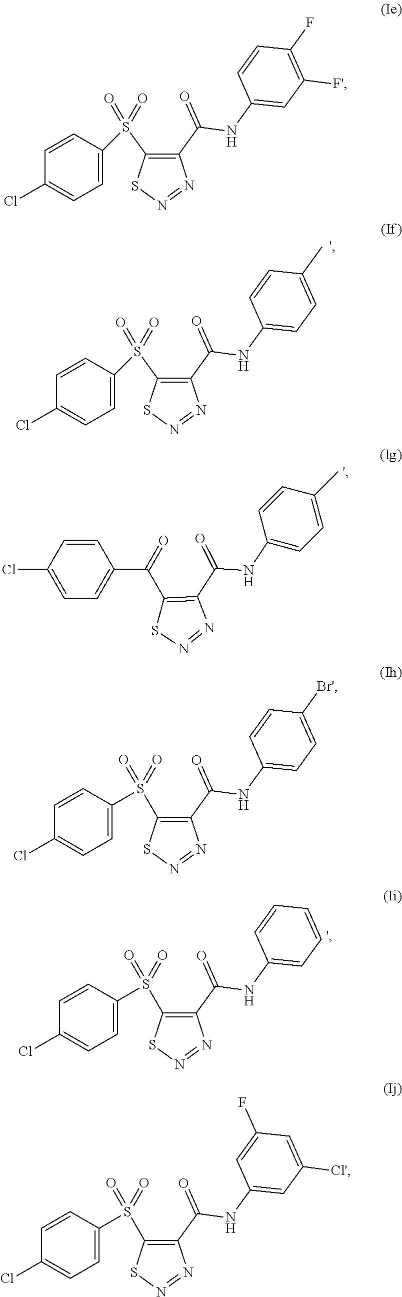 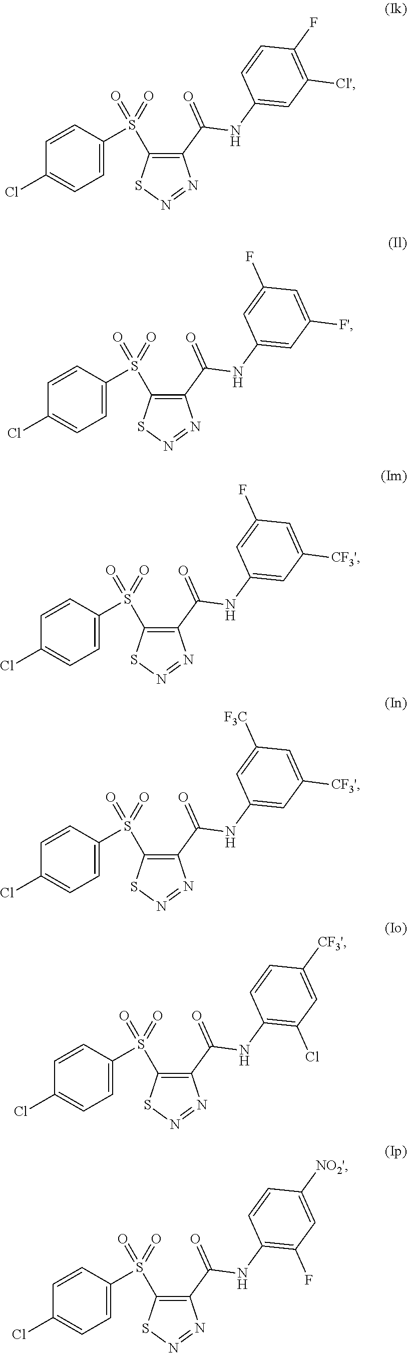 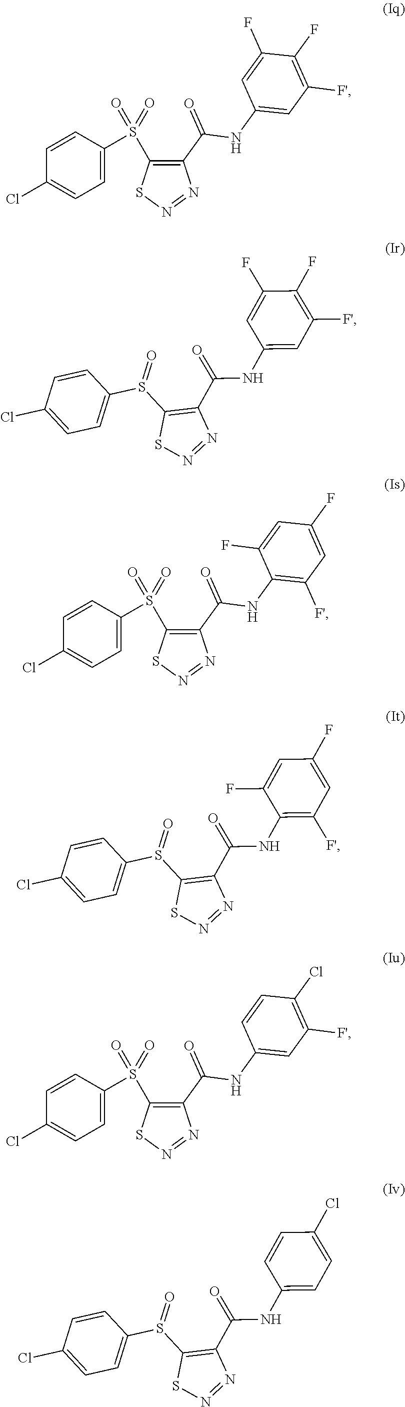 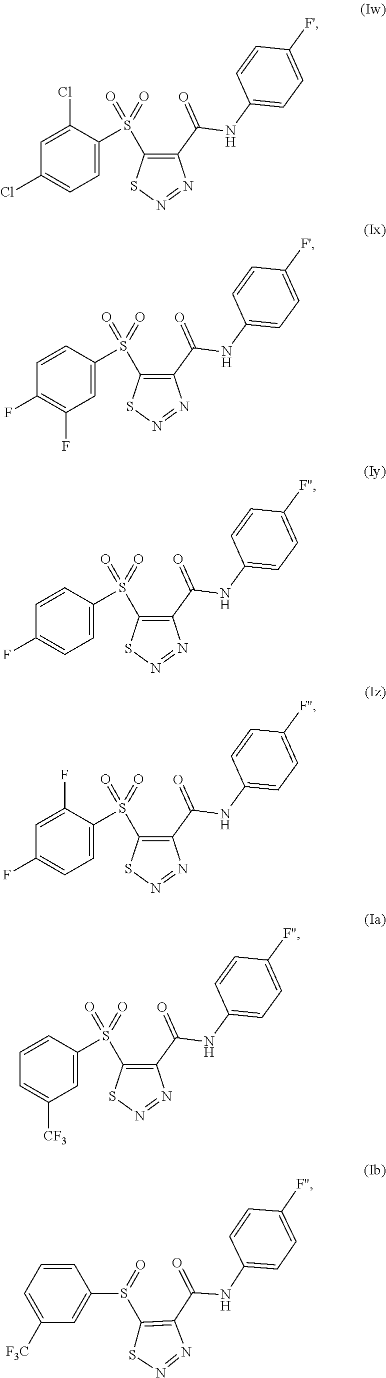 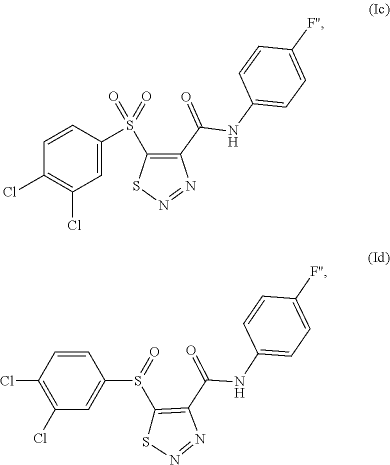
or a pharmaceutically acceptable salt thereof.

33. The method of claim 26, wherein said Prp8 intein activity is splicing.

34. (canceled)

35. The method of claim 26, wherein the cell or tissue is from a subject having a fungal infection or from a subject at risk of having a fungal infection.

36. The method of claim 35, wherein said fungus is selected from one or more of Cryptococcus, Aspergillus, Blastomyces, Coccidiodes, Histoplasma, Phycomyces, Tinea corporis, Tinea unguis, Sporothrix schenckii, Pneumocystis carinii, and Candida.

37. (canceled)

38. The method of claim 26 further comprising:
detecting a Prp8 intein splicing level in said cell or tissue; and
optionally comparing said detected Prp8 intein splicing level in said subject to a Prp8 intein splicing level standard for a subject not having or not at risk of having a fungal infection.

39. (canceled)

40. A method for screening for compounds that inhibit Prp8 intein splicing comprising an assay, said assay comprising:
providing a GFP-Prp8 fusion protein;
treating said GFP-Prp8 fusion protein with an intein splicing buffer;
reacting the treated GFP-Prp8 fusion protein with one or more reagent; and
detecting Prp8 intein splicing activity in a compound.

41. The method of claim 40, wherein
(i) the GFP-Prp8 fusion protein comprises a Prp8 intein, a first GFP residue, and a second GFP residue; and/or
(ii) said one or more reagent is a reducing reagent selected from the group consisting of Tris (2-carboxyethyl) phosphine, lithium aluminum hydride, nascent (atomic) hydrogen, hydrogen without or with a suitable catalyst, sodium amalgam, sodium-lead alloy, zinc amalgam, diborane, sodium borohydride, compounds containing the Fe²⁺ ion and/or an Sn²⁺ ion, sulfur dioxide, sulfite compounds, dithionates, thiosulfates, iodides, hydrogen peroxide, hydrazine, diisobutylaluminum hydride, oxalic acid, formic acid, ascorbic acid, reducing sugars, phosphites, hypophosphites, phosphorous acid, dithiothreitol, carbon monoxide, and cyanides.

42. (canceled)

43. The method of claim 40, wherein the compound treated with the one or more reagent
(i) triggers Prp8 intein splicing; or
(ii) inhibits Prp8 intein splicing.

44-46. (canceled)


SEQUENCE LISTING (7 sequences)

SEQ ID NO: 1 - protein, 7 aa, Artificial Sequence
  modified peptide from Prp8 intein
CLQNGYR

SEQ ID NO: 2 - protein, 10 aa, Artificial Sequence
  modified peptide from Prp8 intein
QASCLQNGYR

SEQ ID NO: 3 - protein, 43 aa, Artificial Sequence
  modified peptide from Prp8 intein
MGSSHHHHHHSSGLVPRGSHMASMTGGQQMGRGSEFELRRQAS

SEQ ID NO: 4 - protein, 28 aa, Artificial Sequence
  modified peptide from Prp8 intein
EVPANSTVLSFCAFAVDAAKAYKDYLAS

SEQ ID NO: 5 - DNA, 48 bp, Artificial Sequence
  primer
ccttgttaatcgtatcgtgttaaaaggttgtctgcagaacggtacccg

SEQ ID NO: 6 - DNA, 57 bp, Artificial Sequence
  primer
cgagaatgtttccatcttctttaaaatctgagttgtgtaataccaaatagtcatgac

SEQ ID NO: 7 - protein, 18 aa, Artificial Sequence
  tag
GHHHHHHEQKLISEEDLG